(19) United States
(12) Patent Application Publication
(10) Pub. No.: US 2026/0176277 A1
(43) Pub. Date: Jun. 25, 2026

(54) COMPOUNDS AND COMPOSITIONS AS eIF4E INHIBITORS AND USES THEREOF

(71) Applicant: Ribometrix, Inc., Durham, NC (US)

(72) Inventors: Brian LAWHORN, Fuquay-Varina, NC (US); Roheeth Kumar PAVANA, Morrisville, NC (US); Stephen THOMSON, Durham, NC (US); Rajeev BHIDE, Princeton, NJ (US); Ashis Kumar SAHA, Framingham, MA (US); Donald L. HERTZOG, Durham, NC (US); Ramamurty VS CHANGALVALA, Morrisville, NC (US)

(21) Appl. No.: 19/128,752

(22) PCT Filed: Nov. 13, 2023
(86) PCT No.: PCT/US2023/079540

§ 371 Date: May 9, 2025

Related U.S. Application Data
(60) Provisional application No. 63/424,462, filed on Nov. 10, 2022.

(51) Int. Cl. C07D 487/04; C07D 401/14; C07D 471/04

(52) U.S. Cl. CPC C07D 487/04; C07D 401/14; C07D 471/04

(57) ABSTRACT

Described herein are compounds of Formula I, and their pharmaceutically acceptable salts, solvates, stereoisomers or prodrugs, as well as their uses (e.g., as eIF4E inhibitors).

### RELATED APPLICATION

This application claims the benefit of and priority to U.S. Provisional Application No. 63/424,462, filed Nov. 10, 2022, the contents of which are incorporated herein by reference in their entirety.

### BACKGROUND

Eukaryotic initiation factor 4E (eIF4E) is the limiting protein factor for initiation of mRNA translation. For example, eIF4E is shown to modulate the translation of cyclin D mRNA. eIF4E initiates translation by binding to the 7-methylguanosine cap at the 5′ end of mRNAs, recruiting the other members of the eIF4F complex, including the scaffolding protein, eIF4G, and the RNA helicase, eIF4A. Once assembled at the 5′ end of mRNAs, the eIF4F complex then recruits additional translation initiation factor factors, culminating in ribosome recruitment and initiation of protein translation.

Under basal conditions, the activity of eIF4E is regulated by multiple mechanisms, including binding to and sequestration by the abundant negative regulatory proteins, the 4E binding proteins (4EBPs). In cancer, eIF4E activity is elevated through several mechanisms including mutational activation of oncogenic signaling pathways such as receptor tyrosine kinases (RTK), RAS/RAF family members, PI3K family members, and others that converge on eIF4E (REF). Dysregulated expression of eIF4E itself is pro-tumorigenic, underscoring its important role in cell transformation and cancer formation. Additionally, elevated expression in patients has been shown to lead to poor prognosis in multiple indications including breast, head and neck, ovarian and colorectal cancers. Up-regulation of eIF4E activity is also reported to be important for evolution of resistance to chemotherapeutic and targeted cancer agents. Lastly, genetic inhibition of eIF4E as well as disruption of other facets of the MNK1-eIF4E axis have demonstrated antitumor efficacy in multiple preclinical models including melanoma, ovarian, esophageal, lung, and breast cancer.

Therefore, pharmacological inhibition of elF4E activity has the potential to be an effective anti-neoplastic therapy.

### SUMMARY

In certain aspects, the present disclosure provides compounds of Formula I:

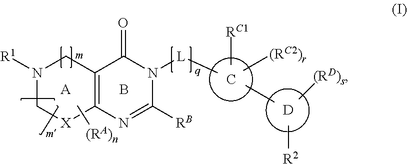

or pharmaceutically acceptable salts, solvates, stereoisomers, or prodrugs thereof,
wherein:
each -L- is independently —O—, —NR^{L}—, —CR^{L1}R^{L2}—, —CR^{L1}—CR^{L2}—, or —C≡C—;
each R^{L} is independently hydrogen or optionally substituted C₁₋₆ alkyl;
each R^{L1} and each R^{L2} is independently hydrogen, halogen, —CN, —NO₂, —OH, —NH₂, C₁₋₆ alkyl, C₁₋₆ alkoxy, or C₁₋₆ alkylamino, wherein the alkyl, alkoxy, or alkylamino is optionally substituted;
q is an integer selected from 1 to 5;
Ring C and Ring D are independently C₆₋₁₀ aryl or 5- to 10-membered heteroaryl;
R^{C1}, each R^{C2}, and each R^{D} are independently halogen, —CN, —NO₂, —OH, —NH₂, C₁₋₆ alkyl, C₂₋₆ alkenyl, C₂₋₆ alkynyl, C₁₋₆ alkoxy, C₁₋₆ alkylamino, C₃₋₆ carbocyclyl, or 3- to 6-membered heterocyclyl, wherein the alkyl, alkenyl, alkynyl, alkoxy, alkylamino, carbocyclyl, or heterocyclyl is optionally substituted;
r and s are independently an integer selected from 0 to 6, as valency permits;
R² is halogen, —CN, —NO₂, —OH, —NH₂, C₁₋₆ alkyl, C₂₋₆ alkenyl, C₂₋₆ alkynyl, C₁₋₆ alkoxy, C₁₋₆ alkylamino, C₆₋₁₀ aryl, 5- to 10-membered heteroaryl, C₃₋₆ carbocyclyl, 3- to 6-membered heterocyclyl, —NR^{c}S(═O)₂R^{a}, —N(S(═O)₂R^{a})₂, —S(═O)₂R^{a}, —S(═O)₂OR^{b}, —S(═O)₂NR^{c}R^{d}, —C(═O)OR^{b}, —C(═O)NR^{c}S(═O)₂R^{a}, or C(═O)NR^{c}R^{d}, wherein the alkyl, alkenyl, alkynyl, alkoxy, alkylamino, carbocyclyl, heterocyclyl, aryl, or heteroaryl is optionally substituted;
R¹ is hydrogen, —CN, C₁₋₆ alkyl, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —(C₁₋₆ alkylene)-(C₆₋₁₀ aryl), (C₁₋₆ alkylene)-(5- to 10-membered heteroaryl), —(C₁₋₆ alkylene)-(C₃₋₁₂ carbocyclyl), —(C₁₋₆ alkylene)-(3- to 12-membered heterocyclyl), —S(═O)R^{a}, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, alkylene, carbocyclyl, heterocyclyl, aryl, or heteroaryl is optionally substituted;
X is —O— or —C(R^{X})₂—;
each R^{X} is independently hydrogen, halogen, —CN, —NO₂, —OH, —NH₂, C₁₋₆ alkyl, C₂₋₆ alkenyl, C₂₋₆ alkynyl, C₁₋₆ alkoxy, C₁₋₆ alkylamino, C₃₋₆ carbocyclyl, or 3- to 6-membered heterocyclyl, wherein the alkyl, alkenyl, alkynyl, alkoxy, alkylamino, carbocyclyl, or heterocyclyl is optionally substituted; or
two geminal R^{X}, together with the carbon atom to which they are connected, form an oxo;
m and m′ are independently an integer selected from 0 to 2;
each R^{A} is independently oxo, halogen, —CN, —NO₂, —OH, —NH₂, C₁₋₆ alkyl, C₂₋₆ alkenyl, C₂₋₆ alkynyl, C₁₋₆ alkoxy, C₁₋₆ alkylamino, C₃₋₆ carbocyclyl, or 3- to 6-membered heterocyclyl, wherein the alkyl, alkenyl, alkynyl, alkoxy, alkylamino, carbocyclyl, or heterocyclyl is optionally substituted;
n is an integer selected from 0 to 10, as valency permits;
R^{B} is hydrogen, halogen, —CN, —NO₂, —OH, —NH₂, C₁₋₆ alkyl, C₂₋₆ alkenyl, C₂₋₆ alkynyl, C₁₋₆ alkoxy, C₁₋₆ alkylamino, C₃₋₆ carbocyclyl, or 3- to 6-membered heterocyclyl, wherein the alkyl, alkenyl, alkynyl, alkoxy, alkylamino, carbocyclyl, or heterocyclyl is optionally substituted;
wherein:
each R^{a} is independently C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl;
each R^{b} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; and
each R^{c} and each R^{d} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; or
R^{c} and R^{d}, together with the nitrogen atom to which they are attached, form 3- to 12-membered heterocyclyl;
wherein each occurrence of R^{a}, R^{b}, R^{c}, and R^{d} is independently and optionally substituted.

In certain aspects, the present disclosure provides pharmaceutical compositions comprising the compound disclosed herein and a pharmaceutically acceptable excipient.

In certain aspects, the present disclosure provides methods of inhibiting a protein in a subject or biological sample comprising administering the compound disclosed herein to the subject or contacting the biological sample with the compound disclosed herein.

In certain aspects, the present disclosure provides uses of the compound disclosed herein in the manufacture of a medicament for inhibiting a protein in a subject or biological sample.

In certain aspects, the present disclosure provides compounds disclosed herein for use in inhibiting a protein in a subject or biological sample.

In certain aspects, the present disclosure provides methods of treating or preventing a disease or disorder a subject in need thereof, comprising administering to the subject the compound disclosed herein.

In certain aspects, the present disclosure provides uses of the compound disclosed herein in the manufacture of a medicament for treating or preventing a disease or disorder in a subject in need thereof.

In certain aspects, the present disclosure provides compounds disclosed herein for use in treating or preventing a disease or disorder in a subject in need thereof.

The details of the disclosure are set forth in the accompanying description below. Although methods and materials similar or equivalent to those described herein can be used in the practice or testing of the present disclosure, illustrative methods and materials are now described. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs.

All publications and patents mentioned herein are hereby incorporated by reference in their entirety as if each individual publication or patent is specifically and individually indicated to be incorporated by reference. In case of conflict, the present application, including any definitions herein, will control.

### DETAILED DESCRIPTION

The present disclosure relates to compounds that inhibit eIF4E activity, as well as pharmaceutical compositions thereof. The present disclosure further relates to methods of inhibiting a protein in a subject or biological sample comprising administering a compound described herein to the subject or contacting the biological sample with a compound described herein. The present disclosure also relates to methods of treating or preventing a disease or disorder a subject in need thereof, comprising administering to the subject a compound described herein.

### Compounds of the Application

In certain aspects, the present disclosure provides a compound of Formula I:

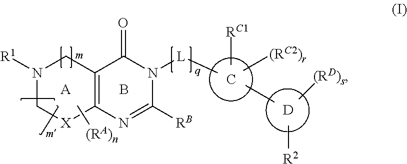

or a pharmaceutically acceptable salt, solvate, stereoisomer, or prodrug thereof,
wherein:
each -L- is independently —O—, —NR^{L}—, —CR^{L1}R^{L2}—, —CR^{L1}═CR^{L2}—, or —C≡C—;
each R^{L} is independently hydrogen or optionally substituted C₁₋₆ alkyl;
each R^{L1} and each R^{L2} is independently hydrogen, halogen, —CN, —NO₂, —OH, —NH₂, C₁₋₆ alkyl, C₁₋₆ alkoxy, or C₁₋₆ alkylamino, wherein the alkyl, alkoxy, or alkylamino is optionally substituted:
q is an integer selected from 1 to 5;
Ring C and Ring D are independently C₆₋₁₀ aryl or 5- to 10-membered heteroaryl;
R^{C1}, each R^{C2}, and each R^{D} are independently halogen, —CN, —NO₂, —OH, —NH₂, C₁₋₆ alkyl, C₂₋₆ alkenyl, C₂₋₆ alkynyl, C₁₋₆ alkoxy, C₁₋₆ alkylamino, C₃₋₆ carbocyclyl, or 3- to 6-membered heterocyclyl, wherein the alkyl, alkenyl, alkynyl, alkoxy, alkylamino, carbocyclyl, or heterocyclyl is optionally substituted;
r and s are independently an integer selected from 0 to 6, as valency permits;
R² is halogen, —CN, —NO₂, —OH, —NH₂, C₁₋₆ alkyl, C₂₋₆ alkenyl, C₂₋₆ alkynyl, C₁₋₆ alkoxy, C₁₋₆ alkylamino, C₆₋₁₀ aryl, 5- to 10-membered heteroaryl, C₃₋₆ carbocyclyl, 3- to 6-membered heterocyclyl, —NR^{c}S(═O)₂R^{a}, —N(S(═O)₂R^{a})₂, —S(═O)₂R^{a}, —S(═O)₂OR^{b}, —S(═O)₂NR^{c}R^{d}, —C(═O)OR^{b}, —C(═O)NR^{c}S(═O)₂R^{a}, or C(═O)NR^{c}R^{d}, wherein the alkyl, alkenyl, alkynyl, alkoxy, alkylamino, carbocyclyl, heterocyclyl, aryl, or heteroaryl is optionally substituted;
R¹ is hydrogen, —CN, C₁₋₆ alkyl, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —(C₁₋₆ alkylene)-(C₆₋₁₀ aryl), (C₁₋₆ alkylene)-(5- to 10-membered heteroaryl), —(C₁₋₆ alkylene)-(C₃₋₁₂ carbocyclyl), —(C₁₋₆ alkylene)-(3- to 12-membered heterocyclyl), —S(═O)R^{a}, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, alkylene, carbocyclyl, heterocyclyl, aryl, or heteroaryl is optionally substituted;
X is —O— or —C(R^{X})₂—;
each R^{X} is independently hydrogen, halogen, —CN, —NO₂, —OH, —NH₂, C₁₋₆ alkyl, C₂₋₆ alkenyl, C₂₋₆ alkynyl, C₁₋₆ alkoxy, C₁₋₆ alkylamino, C₃₋₆ carbocyclyl, or 3- to 6-membered heterocyclyl, wherein the alkyl, alkenyl, alkynyl, alkoxy, alkylamino, carbocyclyl, or heterocyclyl is optionally substituted; or
two geminal R^{X}, together with the carbon atom to which they are connected, form an oxo;
m and m′ are independently an integer selected from 0 to 2;
each R^{A} is independently oxo, halogen, —CN, —NO₂, —OH, —NH₂, C₁₋₆ alkyl, C₂₋₆ alkenyl, C₂₋₆ alkynyl, C₁₋₆ alkoxy, C₁₋₆ alkylamino, C₃₋₆ carbocyclyl, or 3- to 6-membered heterocyclyl, wherein the alkyl, alkenyl, alkynyl, alkoxy, alkylamino, carbocyclyl, or heterocyclyl is optionally substituted;
n is an integer selected from 0 to 10, as valency permits;
R^{B} is hydrogen, halogen, —CN, —NO₂, —OH, —NH₂, C₁₋₆ alkyl, C₂₋₆ alkenyl, C₂₋₆ alkynyl, C₁₋₆ alkoxy, C₁₋₆ alkylamino, C₃₋₆ carbocyclyl, or 3- to 6-membered heterocyclyl, wherein the alkyl, alkenyl, alkynyl, alkoxy, alkylamino, carbocyclyl, or heterocyclyl is optionally substituted;
wherein:
each R^{a} is independently C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl;
each R^{b} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; and
each R^{c} and each R^{d} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; or
R^{c} and R^{d}, together with the nitrogen atom to which they are attached, form 3- to 12-membered heterocyclyl;
wherein each occurrence of R^{a}, R^{b}, R^{c}, and R^{d} is independently and optionally substituted.

In certain embodiments, the compound of Formula I is a compound of Formula I-1-i, I-1-ii, I-1-iii, or I-1-iv:

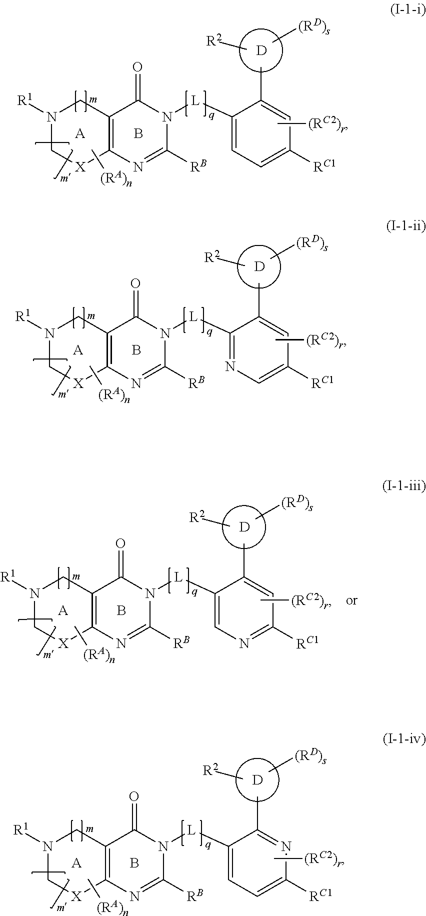

or a pharmaceutically acceptable salt, solvate, stereoisomer, or prodrug thereof.

In certain embodiments, the compound of Formula I is a compound of Formula I-1-i-1, I-1-i-2, I-1-i-3, I-1-iii-1, I-1-iii-2, or I-1-iii-3:

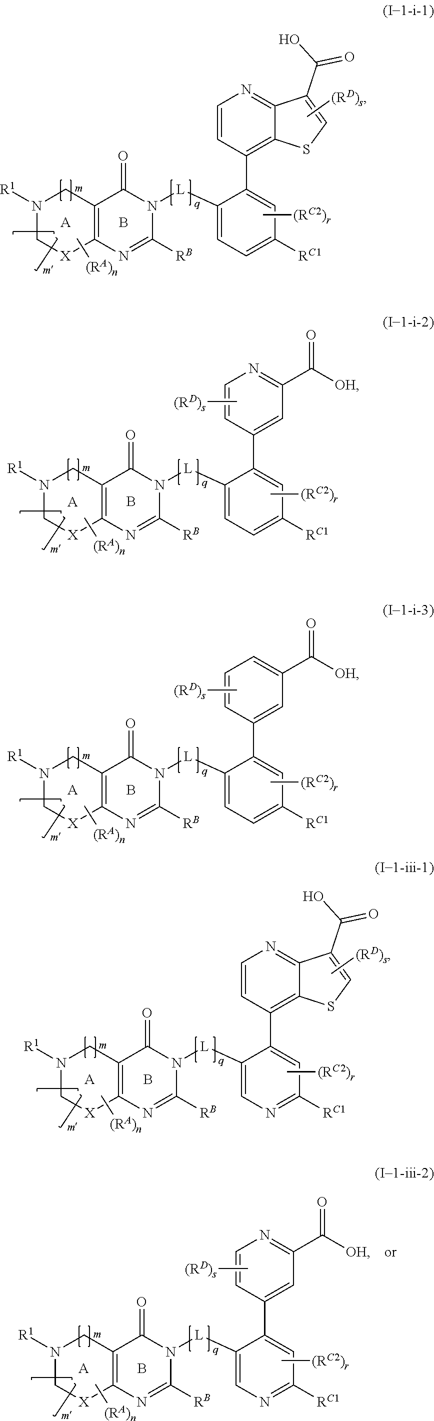

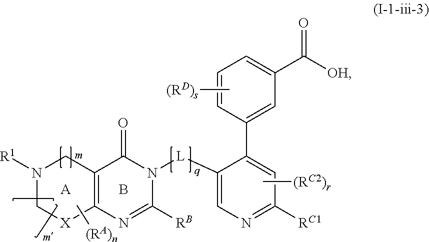

or a pharmaceutically acceptable salt, solvate, stereoisomer, or prodrug thereof.

Embodiments of the variables in any of the Formulae described herein, e.g., Formula I-Formula I-1-iii-3, as applicable, are described below. Any of the variables can be any moiety as described in the embodiments below. Also, any moieties described for any of the variables can be combined, as applicable, with any moieties described for any of the remaining variables.

In certain embodiments, R¹ is hydrogen, —CN, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₅), 2-propynyl (C₃), 1-butynyl (C₄), 2-butynyl (C₄), pentynyl (C₅), or hexynyl (C₆)), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), —(C₁₋₆ alkylene)-(C₆₋₁₀ aryl), —(C₁₋₆ alkylene)-(5- to 10-membered heteroaryl), —(C₁₋₆ alkylene)-(C₃₋₁₂ carbocyclyl), —(C₁₋₆ alkylene)-(3- to 12-membered heterocyclyl), —S(═O)R^{a}, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, alkylene, carbocyclyl, heterocyclyl, aryl, or heteroaryl is optionally substituted. In certain embodiments, the C₁₋₆ alkene is selected from is selected from methylene (—CH₂—), ethylene (—CH₂CH₂—), propylene (—CH₂CH₂CH₂—), butylene (—CH₂CH₂CH₂CH₂—), pentylene (—CH₂CH₂CH₂CH₂CH₂—), and hexylene (—CH₂CH₂CH₂CH₂CH₂CH₂—), In certain embodiments, R¹ is optionally substituted with one or more R^{u}.

In certain embodiments, R¹ is hydrogen, —CN, C₁₋₆ alkyl, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —(C₁₋₆ alkylene)-(C₆₋₁₀ aryl), —(C₁₋₆ alkylene)-(5- to 10-membered heteroaryl), —(C₁₋₆ alkylene)-(C₃₋₁₂ carbocyclyl), —(C₁₋₆ alkylene)-(3- to 12-membered heterocyclyl), —S(═O)R^{a}, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, alkylene, carbocyclyl, heterocyclyl, aryl, or heteroaryl is optionally substituted. In certain embodiments, R¹ is optionally substituted with one or more R^{u}.

In certain embodiments, R¹ is hydrogen, —CN, C₁₋₆ alkyl, C₆₋₁₀ aryl, 5- to 10-membered heteroaryl, C₃₋₁₂ carbocyclyl, 3- to 12-membered heterocyclyl, —(C₁₋₆ alkylene)-(C₆₋₁₀ aryl), —(C₁₋₆ alkylene)-(5- to 10-membered heteroaryl), —(C₁₋₆ alkylene)-(C₃₋₁₂ carbocyclyl), —(C₁₋₆ alkylene)-(3- to 12-membered heterocyclyl), —S(═O)R^{a}, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, alkylene, carbocyclyl, heterocyclyl, aryl, or heteroaryl is optionally substituted. In certain embodiments, R¹ is optionally substituted with one or more R^{u}.

In certain embodiments, R¹ is —CN, C₁₋₆ alkyl, C₆₋₁₀ aryl, 5- to 10-membered heteroaryl, 3- to 12-membered heterocyclyl, —(C₁₋₆ alkylene)-(C₆₋₁₀ aryl), —(C₁₋₆ alkylene)-(5- to 10-membered heteroaryl), —C(═O)R^{a}, wherein the alkyl, alkylene, heterocyclyl, aryl, or heteroaryl is optionally substituted. In certain embodiments, R¹ is optionally substituted with one or more R^{u}.

In certain embodiments, R¹ is optionally substituted C₁₋₆ alkyl. In certain embodiments, R¹ is optionally substituted C₆₋₁₀ aryl. In certain embodiments, R¹ is optionally substituted 5- to 10-membered heteroaryl comprising one or two 5- or 6-membered rings and 1-5 heteroatoms selected from N, O, and S. In certain embodiments, R¹ is optionally substituted C₃₋₁₂ carbocyclyl. In certain embodiments, R¹ is optionally substituted 3- to 12-membered heterocyclyl comprising one or two 3- to 8-membered rings and 1-5 heteroatoms selected from N, O, and S. In certain embodiments, R¹ is —(C₁₋₆ alkylene)-(C₆₋₁₀ aryl), wherein the alkylene or aryl is optionally substituted. In certain embodiments, R¹ is —(C₁₋₆ alkylene)-(5- to 10-membered heteroaryl), wherein the heteroaryl comprises one or two 5- or 6-membered rings and 1-5 heteroatoms selected from N, O, and S, wherein the alkylene or heteroaryl is optionally substituted. In certain embodiments, R¹ is —(C₁₋₆ alkylene)-(C₃₋₁₂ carbocyclyl), wherein the alkylene or carbocyclyl is optionally substituted. In certain embodiments, R¹ is —(C₁₋₆ alkylene)-(3- to 12-membered heterocyclyl), wherein the heterocyclyl comprises one or two 3- to 8-membered rings and 1-5 heteroatoms selected from N, O, and S, wherein the alkylene or heterocyclyl is optionally substituted. In certain embodiments, R¹ is optionally substituted with one or more R^{u}.

In certain embodiments, R¹ is optionally substituted C₆₋₁₀ aryl. In certain embodiments, the aryl is optionally substituted with one or more R^{u}.

In certain embodiments, R¹ is optionally substituted 5- to 10-membered heteroaryl comprising one or two 5- or 6-membered rings and 1-5 heteroatoms selected from N, O, and S. In certain embodiments, R¹ is optionally substituted heteroaryl comprising one 5- or 6-membered ring and 1-3 heteroatoms selected from N, O, and S. In certain embodiments, R¹ is optionally substituted heteroaryl comprising one 5-membered ring and 1-3 heteroatoms selected from N, O, and S. In certain embodiments, R¹ is optionally substituted heteroaryl comprising one 6-membered ring and 1-3 heteroatoms selected from N, O, and S. In certain embodiments, R¹ is optionally substituted heteroaryl comprising two 5- or 6-membered rings and 1-5 heteroatoms selected from N, O, and S. In certain embodiments, R¹ is optionally substituted heteroaryl comprising two 5-membered rings and 1-5 heteroatoms selected from N, O, and S. In certain embodiments, R¹ is optionally substituted heteroaryl comprising two 6-membered rings and 1-5 heteroatoms selected from N, O, and S. In certain embodiments, R¹ is optionally substituted heteroaryl comprising one 5-membered ring and one 6-membered ring and 1-5 heteroatoms selected from N, O, and S. In certain embodiments, the heteroaryl is optionally substituted with one or more R^{u}.

In certain embodiments, R¹ is optionally substituted C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀),). In certain embodiments, the carbocyclyl is optionally substituted with one or more R^{u}.

In certain embodiments, R¹ is optionally substituted 3- to 12-membered heterocyclyl comprising one or two 3- to 8-membered rings and 1-5 heteroatoms selected from N, O, and S. In certain embodiments, R¹ is optionally substituted heterocyclyl comprising one 3- to 8-membered ring and 1-3 heteroatoms selected from N, O, and S. In certain embodiments, R¹ is optionally substituted heterocyclyl comprising one 3-membered ring and 1-3 heteroatoms selected from N, O, and S. In certain embodiments, R¹ is optionally substituted heterocyclyl comprising one 4-membered ring and 1-3 heteroatoms selected from N, O, and S. In certain embodiments, R¹ is optionally substituted heterocyclyl comprising one 5-membered ring and 1-3 heteroatoms selected from N, O, and S. In certain embodiments, R¹ is optionally substituted heterocyclyl comprising one 6-membered ring and 1-4 heteroatoms selected from N, O, and S. In certain embodiments, R¹ is optionally substituted heterocyclyl comprising one 7-membered ring and 1-4 heteroatoms selected from N, O, and S. In certain embodiments, R¹ is optionally substituted heterocyclyl comprising one 8-membered ring and 1-5 heteroatoms selected from N, O, and S. In certain embodiments, R¹ is optionally substituted heterocyclyl comprising two 3- to 8-membered rings and 1-5 heteroatoms selected from N, O, and S. In certain embodiments, R¹ is optionally substituted heterocyclyl comprising two 5-membered rings and 1-5 heteroatoms selected from N, O, and S. In certain embodiments, R¹ is optionally substituted heterocyclyl comprising two 6-membered rings and 1-5 heteroatoms selected from N, O, and S. In certain embodiments, R¹ is optionally substituted heterocyclyl comprising one 5-membered ring and one 6-membered ring and 1-5 heteroatoms selected from N, O, and S. In certain embodiments, the heterocyclyl is optionally substituted with one or more R^{u}.

In certain embodiments, R¹ is —(C₁₋₆ alkylene)-(C₆₋₁₀ aryl), wherein the alkylene or aryl is optionally substituted. In certain embodiments, the aryl is optionally substituted with one or more R^{u}.

In certain embodiments, R¹ is —(C₁₋₆ alkylene)-(5- to 10-membered heteroaryl), wherein the heteroaryl comprises one or two 5- or 6-membered rings and 1-5 heteroatoms selected from N, O, and S, wherein the alkylene or heteroaryl is optionally substituted. In certain embodiments, the optionally substituted heteroaryl comprises one 5- or 6-membered ring and 1-3 heteroatoms selected from N, O, and S. In certain embodiments, the optionally substituted heteroaryl comprises one 5-membered ring and 1-3 heteroatoms selected from N, O, and S. In certain embodiments, the optionally substituted heteroaryl comprises one 6-membered ring and 1-3 heteroatoms selected from N, O, and S. In certain embodiments, the optionally substituted heteroaryl comprises two 5- or 6-membered rings and 1-5 heteroatoms selected from N, O, and S. In certain embodiments, the optionally substituted heteroaryl comprises two 5-membered rings and 1-5 heteroatoms selected from N, O, and S. In certain embodiments, the optionally substituted heteroaryl comprises two 6-membered rings and 1-5 heteroatoms selected from N, O, and S. In certain embodiments, the optionally substituted heteroaryl comprises one 5-membered ring and one 6-membered ring and 1-5 heteroatoms selected from N, O, and S. In certain embodiments, the heteroaryl is optionally substituted with one or more R^{u}.

In certain embodiments, R¹ is —(C₁₋₆ alkylene)-(C₃₋₁₂ carbocyclyl), wherein the C₃₋₁₂ carbocyclyl is selected from cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₅), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), and spiro[4.5]decanyl (C₁₀), wherein the alkylene or carbocyclyl is optionally substituted. In certain embodiments, the carbocyclyl is optionally substituted with one or more R″.

In certain embodiments, R¹ is —(C₁₋₆ alkylene)-(3- to 12-membered heterocyclyl), wherein the heterocyclyl comprises one or two 3- to 8-membered rings and 1-5 heteroatoms selected from N, O, and S, wherein the alkylene or heterocyclyl is optionally substituted. In certain embodiments, the optionally substituted heterocyclyl comprises one 3- to 8-membered ring and 1-3 heteroatoms selected from N, O, and S. In certain embodiments, the optionally substituted heterocyclyl comprises one 3-membered ring and 1-3 heteroatoms selected from N, O, and S. In certain embodiments, the optionally substituted heterocyclyl comprises one 4-membered ring and 1-3 heteroatoms selected from N, O, and S. In certain embodiments, the optionally substituted heterocyclyl comprises one 5-membered ring and 1-3 heteroatoms selected from N, O, and S. In certain embodiments, the optionally substituted heterocyclyl comprises one 6-membered ring and 1-4 heteroatoms selected from N, O, and S. In certain embodiments, the optionally substituted heterocyclyl comprises one 7-membered ring and 1-4 heteroatoms selected from N, O, and S. In certain embodiments, the optionally substituted heterocyclyl comprises one 8-membered ring and 1-5 heteroatoms selected from N, O, and S. In certain embodiments, the optionally substituted heterocyclyl comprises two 3- to 8-membered rings and 1-5 heteroatoms selected from N, O, and S. In certain embodiments, the optionally substituted heterocyclyl comprises two 5-membered rings and 1-5 heteroatoms selected from N, O, and S. In certain embodiments, the optionally substituted heterocyclyl comprises two 6-membered rings and 1-5 heteroatoms selected from N, O, and S. In certain embodiments, the optionally substituted heterocyclyl comprises one 5-membered ring and one 6-membered ring and 1-5 heteroatoms selected from N, O, and S. In certain embodiments, the heterocyclyl is optionally substituted with one or more R^{u}.

In certain embodiments, m is 0. In certain embodiments, m is 1. In certain embodiments, m is 2.

In certain embodiments, m′ is 0. In certain embodiments, m′ is 1. In certain embodiments, m′ is 2.

In certain embodiments, m is 0) and m′ is 0. In certain embodiments, m is 0 and m′ is 1. In certain embodiments, m is 0 and m′ is 2. In certain embodiments, m is 1 and m′ is 0. In certain embodiments, m is 1 and m′ is 1. In certain embodiments, m is 1 and m′ is 2. In certain embodiments, m is 2 and m′ is 0. In certain embodiments, m is 2 and m′ is 1. In certain embodiments, m is 2 and m′ is 2.

In certain embodiments, each R^{A} is independently oxo, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)), or 3- to 6-membered heterocyclyl (e.g., heterocyclyl comprising one 3- to 6-membered ring and 1-3 heteroatoms selected from N, O, and S), wherein the alkyl, alkenyl, alkynyl, alkoxy, alkylamino, carbocyclyl, or heterocyclyl is optionally substituted. In certain embodiments, each R^{A} is independently optionally substituted with one or more R^{u}.

In certain embodiments, each R^{A} is independently oxo, halogen, —CN, —NO₂, —OH, —NH₂, C₁₋₆ alkyl, C₂₋₆ alkenyl, C₂₋₆ alkynyl, C₁₋₆ alkoxy, or C₁₋₆ alkylamino, wherein the alkyl, alkenyl, alkynyl, alkoxy, or alkylamino is optionally substituted. In certain embodiments, each R^{A} is independently optionally substituted with one or more R^{u}.

In certain embodiments, each R^{A} is independently halogen, —CN, —NO₂, —OH, —NH₂, C₁₋₆ alkyl, C₁₋₆ alkoxy, or C₁₋₆ alkylamino, wherein the alkyl, alkoxy, or alkylamino is optionally substituted. In certain embodiments, each R^{A} is independently optionally substituted with one or more R^{u}.

In certain embodiments, n is 0. In certain embodiments, n is 1. In certain embodiments, n is 2. In certain embodiments, n is 3. In certain embodiments, n is 4. In certain embodiments, n is 5. In certain embodiments, n is 6. In certain embodiments, n is 7. In certain embodiments, n is 8. In certain embodiments, n is 9. In certain embodiments, n is 10.

In certain embodiments, R^{B} is hydrogen, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)), or 3- to 6-membered heterocyclyl (e.g., heterocyclyl comprising one 3- to 6-membered ring and 1-3 heteroatoms selected from N, O, and S), wherein the alkyl, alkenyl, alkynyl, alkoxy, alkylamino, carbocyclyl, or heterocyclyl is optionally substituted. In certain embodiments, each R^{B} is independently optionally substituted with one or more R^{u}.

In certain embodiments, R^{B} is hydrogen, halogen, —CN, —NO₂, —OH, —NH₂, C₁₋₆ alkyl, C₂₋₆ alkenyl, C₂₋₆ alkynyl, C₁₋₆ alkoxy, or C₁₋₆ alkylamino, wherein the alkyl, alkenyl, alkynyl, alkoxy, or alkylamino is optionally substituted. In certain embodiments, R^{B} is independently optionally substituted with one or more R^{u}.

In certain embodiments, R^{B} is hydrogen, halogen, —CN, —NO₂, —OH, —NH₂, C₁₋₆ alkyl, C₁₋₆ alkoxy, or C₁₋₆ alkylamino, wherein the alkyl, alkoxy, or alkylamino is optionally substituted. In certain embodiments, R^{B} is independently optionally substituted with one or more R^{u}.

In certain embodiments, R^{B} is hydrogen or optionally substituted C₁₋₆ alkyl. In certain embodiments, R^{B} is hydrogen. In certain embodiments, R^{B} is optionally substituted C₁₋₆ alkyl. In certain embodiments, R^{B} is optionally substituted with one or more R^{u}.

In certain embodiments, Ring C is C₆₋₁₀ aryl or 5- to 10-membered heteroaryl.

In certain embodiments, Ring C is C₆₋₁₀ aryl (e.g., phenyl or naphthyl).

In certain embodiments, Ring C is 5- to 10-membered heteroaryl comprising one or two 5- or 6-membered rings and 1-5 heteroatoms selected from N, O, and S. In certain embodiments, Ring C is 5- to 10-membered heteroaryl comprising one 5- or 6-membered ring and 1-3 heteroatoms selected from N, O, and S. In certain embodiments, Ring C is 5- to 10-membered heteroaryl comprising one 5-membered ring and 1-3 heteroatoms selected from N, O, and S. In certain embodiments, Ring C is 5- to 10-membered heteroaryl comprising one 6-membered ring and 1-3 heteroatoms selected from N, O, and S. In certain embodiments, Ring C is 5- to 10-membered heteroaryl comprising two 5- or 6-membered rings and 1-5 heteroatoms selected from N, O, and S. In certain embodiments, Ring C is 5- to 10-membered heteroaryl comprising two 5-membered rings and 1-5 heteroatoms selected from N, O, and S. In certain embodiments, Ring C is 5- to 10-membered heteroaryl comprising two 6-membered rings and 1-5 heteroatoms selected from N, O, and S. In certain embodiments, Ring C is 5- to 10-membered heteroaryl comprising one 5-membered ring and one 6-membered ring and 1-5 heteroatoms selected from N, O, and S.

In certain embodiments, Ring C is phenyl or pyridinyl.

In certain embodiments, R^{C1} is halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)), or 3- to 6-membered heterocyclyl (e.g., heterocyclyl comprising one 3- to 6-membered ring and 1-3 heteroatoms selected from N, O, and S), wherein the alkyl, alkenyl, alkynyl, alkoxy, alkylamino, carbocyclyl, or heterocyclyl is optionally substituted. In certain embodiments, R^{C1} is optionally substituted with one or more R^{u}.

In certain embodiments, R^{C1} is halogen, —CN, —NO₂, —OH, —NH₂, C₁₋₆ alkyl, C₁₋₆ alkoxy, or C₁₋₆ alkylamino, wherein the alkyl, alkoxy, or alkylamino is optionally substituted. In certain embodiments, R^{C1} is optionally substituted with one or more R^{u}.

In certain embodiments, R^{C1} is halogen (e.g., —F, —Cl, —Br, or —I). In certain embodiments, R^{C1} is —Cl. In certain embodiments, R^{C1} is optionally substituted C₁₋₆ alkyl (e.g., methyl, ethyl, propyl, i-propyl, n-butyl, i-butyl, s-butyl, t-butyl, pentyl, or hexyl). In certain embodiments, R^{C1} is optionally substituted with one or more R^{u}.

In certain embodiments, each R^{C2} is independently halogen (e.g., F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)), or 3- to 6-membered heterocyclyl (e.g., heterocyclyl comprising one 3- to 6-membered ring and 1-3 heteroatoms selected from N, O, and S), wherein the alkyl, alkenyl, alkynyl, alkoxy, alkylamino, carbocyclyl, or heterocyclyl is optionally substituted. In certain embodiments, each R^{C2} is independently optionally substituted with one or more R^{u}.

In certain embodiments, each R^{C2} is independently halogen, —CN, —NO₂, —OH, —NH₂, C₁₋₆ alkyl, C₁₋₆ alkoxy, or C₁₋₆ alkylamino, wherein the alkyl, alkoxy, or alkylamino is optionally substituted. In certain embodiments, each R^{C2} is independently optionally substituted with one or more R^{u}.

In certain embodiments, r is 0. In certain embodiments, r is 1. In certain embodiments, r is 2. In certain embodiments, r is 3. In certain embodiments, r is 4. In certain embodiments, r is 5. In certain embodiments, r is 6.

In certain embodiments, Ring D is C₆₋₁₀ aryl or 5- to 10-membered heteroaryl.

In certain embodiments, Ring D is C₆₋₁₀ aryl (e.g., phenyl or naphthyl).

In certain embodiments, Ring D is 5- to 10-membered heteroaryl comprising one or two 5- or 6-membered rings and 1-5 heteroatoms selected from N, O, and S. In certain embodiments, Ring D is 5- to 10-membered heteroaryl comprising one 5- or 6-membered ring and 1-3 heteroatoms selected from N, O, and S. In certain embodiments, Ring D is 5- to 10-membered heteroaryl comprising one 5-membered ring and 1-3 heteroatoms selected from N, O, and S. In certain embodiments, Ring D is 5- to 10-membered heteroaryl comprising one 6-membered ring and 1-3 heteroatoms selected from N, O, and S. In certain embodiments, Ring D is 5- to 10-membered heteroaryl comprising two 5- or 6-membered rings and 1-5 heteroatoms selected from N, O, and S. In certain embodiments, Ring D is 5- to 10-membered heteroaryl comprising two 5-membered rings and 1-5 heteroatoms selected from N, O, and S. In certain embodiments, Ring D is 5- to 10-membered heteroaryl comprising two 6-membered rings and 1-5 heteroatoms selected from N, O, and S. In certain embodiments, Ring D is 5- to 10-membered heteroaryl comprising one 5-membered ring and one 6-membered ring and 1-5 heteroatoms selected from N, O, and S.

In certain embodiments, Ring D is phenyl, pyridinyl, pyrrolopyridazinyl, or thienopyridinyl.

In certain embodiments, R² is hydrogen, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)), 3- to 6-membered heterocyclyl (e.g., heterocyclyl comprising one 3- to 6-membered ring and 1-3 heteroatoms selected from N, O, and S), —NR^{c}S(═O)₂R^{a}, —N(S(═O)₂R^{a}) 2, —S(═O)₂R^{a}, —S(═O)₂OR^{b}, —S(═O)₂NR^{c}R^{d}, —C(═O)OR^{b}, —C(═O)NR^{c}S(═O)₂R^{a}, or C(═O)NR^{c}R^{d}, wherein the alkyl, alkenyl, alkynyl, alkoxy, alkylamino, carbocyclyl, heterocyclyl, aryl, or heteroaryl is optionally substituted. In certain embodiments, R² is optionally substituted with one or more R^{u}.

In certain embodiments, R² is halogen, —CN, —NO₂, —OH, —NH₂, C₁₋₆ alkyl, C₂₋₆ alkenyl, C₂₋₆ alkynyl, C₁₋₆ alkoxy, C₁₋₆ alkylamino, C₆₋₁₀ aryl, 5- to 10-membered heteroaryl, C₃₋₆ carbocyclyl, 3- to 6-membered heterocyclyl, —NR^{c}S(═O)₂R^{a}, —N(S(═O)₂R^{a})₂, —S(═O)₂R^{a}, —S(═O)₂OR^{b}, —S(═O)₂NR^{c}R^{d}, —C(═O)OR^{b}, —C(═O)NR^{c}S(═O)₂R^{a}, or C(═O)NR^{c}R^{d}, wherein the alkyl, alkenyl, alkynyl, alkoxy, alkylamino, carbocyclyl, heterocyclyl, aryl, or heteroaryl is optionally substituted. In certain embodiments, R² is optionally substituted with one or more R^{u}.

In certain embodiments, R² is halogen, —CN, —NO₂, —OH, —NH₂, C₁₋₆ alkyl, C₂₋₆ alkenyl, C₂₋₆ alkynyl, C₁₋₆ alkoxy, C₁₋₆ alkylamino, —NR^{c}S(═O)₂R^{a}, —N(S(═O)₂R^{a})₂, —S(═O)₂R^{a}, —S(═O)₂OR^{b}, —S(═O)₂NR^{c}R^{d}, —C(═O)OR^{b}, —C(═O)NR^{c}S(═O)₂R^{a}, or C(═O)NR^{c}R^{d}, wherein the alkyl, alkenyl, alkynyl, alkoxy, or alkylamino is optionally substituted. In certain embodiments, R² is optionally substituted with one or more R^{u}.

In certain embodiments, R² is halogen, —CN, —NO₂, —OH, —NH₂, C₁₋₆ alkyl, C₁₋₆ alkoxy, C₁₋₆ alkylamino, —NR^{c}S(═O)₂R^{a}, —N(S(═O)₂R^{a})₂, —S(═O)₂R^{a}, —S(═O)₂OR^{b}, —S(═O)₂NR^{c}R^{d}, —C(═O)OR^{b}, —C(═O)NR^{c}S(═O)₂R^{a}, or C(═O)NR^{c}R^{d}, wherein the alkyl, alkoxy, or alkylamino is optionally substituted. In certain embodiments, R² is optionally substituted with one or more R^{u}.

In certain embodiments, R² is halogen, C₁₋₆ alkyl, C₆₋₁₀ aryl, 5- to 10-membered heteroaryl, C₃₋₆ carbocyclyl, 3- to 6-membered heterocyclyl, —NR^{c}S(═O)₂R^{a}, —N(S(═O)₂R^{a})₂, —S(═O)₂R^{a}, —S(═O)₂OR^{b}, —S(═O)₂NR^{c}R^{d}, —C(═O)OR^{b}, —C(═O)NR^{c}S(═O)₂R^{a}, or C(═O)NR^{c}R^{d}, wherein the alkyl, carbocyclyl, heterocyclyl, aryl, or heteroaryl is optionally substituted. In certain embodiments, R² is optionally substituted with one or more R^{u}.

In certain embodiments, R² is C₁₋₆ alkyl, 5- to 10-membered heteroaryl, —NR^{c}S(═O)₂R^{a}, —N(S(═O)₂R^{a})₂, —C(═O)OR^{b}, —C(═O)NR^{c}S(═O)₂R^{a}, or —C(═O)NR^{c}R^{d}, wherein the alkyl or heteroaryl is optionally substituted. In certain embodiments, R² is optionally substituted with one or more R^{u}.

In certain embodiments, R² is —C(═O)NR^{c}S(═O)R^{a}, or —C(═O)OR^{b}. In certain embodiments, R² is —C(═O)NHS(═O)₂CH₃ or —COOH.

In certain embodiments, each R^{D} is independently halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)), or 3- to 6-membered heterocyclyl (e.g., heterocyclyl comprising one 3- to 6-membered ring and 1-3 heteroatoms selected from N, O, and S), wherein the alkyl, alkenyl, alkynyl, alkoxy, alkylamino, carbocyclyl, or heterocyclyl is optionally substituted. In certain embodiments, each R^{D} is independently optionally substituted with one or more R^{u}.

In certain embodiments, each R^{D} is independently halogen, —CN, —NO₂, —OH, —NH₂, C₁₋₆ alkyl, C₁₋₆ alkoxy, or C₁₋₆ alkylamino, wherein the alkyl, alkoxy, or alkylamino is optionally substituted. In certain embodiments, each R^{D} is independently optionally substituted with one or more R^{u}.

In certain embodiments, each R^{D} is independently halogen or optionally substituted C₁₋₆ alkyl. In certain embodiments, at least one R^{D} is halogen. In certain embodiments, each R^{D} is independently halogen. In certain embodiments, at least one R^{D} is optionally substituted C₁₋₆ alkyl. In certain embodiments, each R^{D} is independently optionally substituted C₁₋₆ alkyl. In certain embodiments, each R^{D} is independently optionally substituted with one or more R^{u}.

In certain embodiments, s is 0. In certain embodiments, s is 1. In certain embodiments, s is 2. In certain embodiments, s is 3. In certain embodiments, s is 4. In certain embodiments, s is 5. In certain embodiments, s is 6.

In certain embodiments, each -L- is independently —O—, —NR^{L}—, —CR^{L1}R^{L2}—, —CR^{L1}═CR^{L2}—, or —C≡C—. In certain embodiments, each -L- is independently —O—, —CR^{L1}R^{L2}—, or —C≡C—.

In certain embodiments, [L]_{q} is

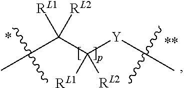

wherein:
* denotes attachment to Ring B, and ** denotes attachment to Ring C;
p is an integer selected from 0 to 3; and
Y is —O—, —CR^{L1}R^{L2}—, or —C≡C—.

In certain embodiments, L is Y.

In certain embodiments, each R^{L1} and each R^{L2} is independently hydrogen, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), or C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), wherein the alkyl, alkoxy, or alkylamino is optionally substituted. In certain embodiments, each R^{L1} and each R^{L2} is independently optionally substituted with one or more R^{u}.

In certain embodiments, each R^{L1} and each R^{L2} is hydrogen.

In certain embodiments, p is 0. In certain embodiments, p is 1. In certain embodiments, p is 2. In certain embodiments, p is 3.

In certain embodiments, Y is —O—. In certain embodiments, Y is —NR^{L}—. In certain embodiments, Y is —CR^{L1}R^{L2}—. In certain embodiments, Y is —C≡C—.

In certain embodiments, X is —O—. In certain embodiments, X is —C(R^{X})₂—.

In certain embodiments, each R^{X} is independently hydrogen, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)), or 3- to 6-membered heterocyclyl (e.g., heterocyclyl comprising one 3- to 6-membered ring and 1-3 heteroatoms selected from N, O, and S), wherein the alkyl, alkenyl, alkynyl, alkoxy, alkylamino, carbocyclyl, or heterocyclyl is optionally substituted. In certain embodiments, each R^{X} is independently optionally substituted with one or more R^{u}.

In certain embodiments, each R^{X} is independently hydrogen, halogen, —CN, —NO₂, —OH, —NH₂, C₁₋₆ alkyl, C₁₋₆ alkoxy, or C₁₋₆ alkylamino, wherein the alkyl, alkoxy, or alkylamino is optionally substituted. In certain embodiments, each R^{X} is independently optionally substituted with one or more R^{u}.

In certain embodiments, each R^{X} is hydrogen.

In certain embodiments, each R^{X} is independently halogen or optionally substituted C₁₋₆ alkyl. In certain embodiments, at least one R^{X} is halogen. In certain embodiments, each R^{X} is independently halogen. In certain embodiments, at least one R^{X} is optionally substituted C₁₋₆ alkyl. In certain embodiments, each R^{X} is independently optionally substituted C₁₋₆ alkyl. In certain embodiments, each R^{X} is independently optionally substituted with one or more R^{u}.

In certain embodiments, each occurrence of R^{a}, R^{b}, R^{c}, and R^{d} is independently and optionally substituted. In certain embodiments, each occurrence of R^{a}, R^{b}, R^{c}, and R^{d} is independently optionally substituted with one or more R^{u}.

In certain embodiments, each R^{a} is independently C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl, or 5- to 10-membered heteroaryl, wherein the alkyl, alkenyl, alkynyl, carbocyclyl, heterocyclyl, aryl, or heteroaryl is optionally substituted with one or more R^{u}.

In certain embodiments, each R^{a} is independently C₁₋₆ alkyl, C₂₋₆ alkenyl, or C₂₋₆ alkynyl, wherein the alkyl, alkenyl, or alkyny, is optionally substituted with one or more R^{u}.

In certain embodiments, each R^{b} is independently hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₅), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl, or 5- to 10-membered heteroaryl, wherein the alkyl, alkenyl, alkynyl, carbocyclyl, heterocyclyl, aryl, or heteroaryl is optionally substituted with one or more R^{u}.

In certain embodiments, each R^{b} is independently C₁₋₆ alkyl, C₂₋₆ alkenyl, or C₂₋₆ alkynyl, wherein the alkyl, alkenyl, or alkyny, is optionally substituted with one or more R^{u}.

In certain embodiments, each R^{c} and each R^{d} is independently hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₅), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl, or 5- to 10-membered heteroaryl, wherein the alkyl, alkenyl, alkynyl, carbocyclyl, heterocyclyl, aryl, or heteroaryl is optionally substituted with one or more R^{u}.

In certain embodiments, each R^{c} and each R^{d} is independently hydrogen, C₁₋₆ alkyl, C₂₋₆ alkenyl, C₂₋₆ alkynyl, wherein the alkyl, alkenyl, or alkynyl is optionally substituted with one or more R^{u}.

In certain embodiments, R^{c} and R^{d}, together with the nitrogen atom to which they are attached, form 3- to 12-membered heterocyclyl (e.g., heterocyclyl comprising one or two 3- to 8-membered rings and 1-5 heteroatoms selected from N, O, and S), wherein the heterocyclyl is optionally substituted with one or more R^{u}.

In certain embodiments, each R^{u} is independently oxo, halogen, —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-1-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, wherein the alkyl, alkoxy, alkylamino, alkenyl, alkynyl, carbocyclyl or heterocyclyl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, each R^{u} is independently oxo, halogen, —CN, —NO₂, —OH, —NH₂, C₁₋₆ alkyl, C₁₋₆ alkoxy, C₁₋₆ alkylamino, C₃₋₆ carbocyclyl, 3- to 6-membered heterocyclyl, wherein the alkyl, alkoxy, alkylamino, carbocyclyl or heterocyclyl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, the compound disclosed herein is selected from the compounds in Table 1 and pharmaceutically acceptable salts, solvates, stereoisomers, or prodrugs thereof.

TABLE 1

| Compound | |
|---|---|
| No. | Chemical Structure |
| 1 | 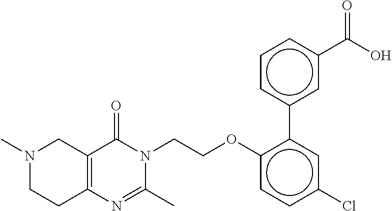 |
| 2 | 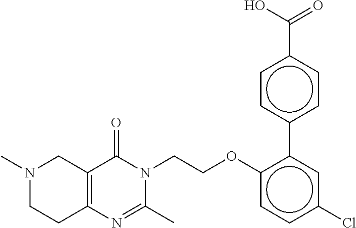 |
| 3 | 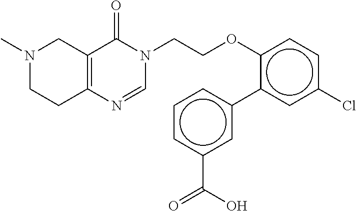 |
| 4 | 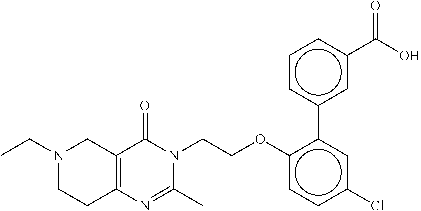 |
| 5 | 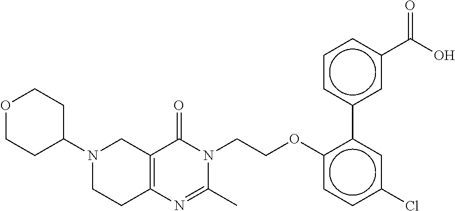 |
| 6 | 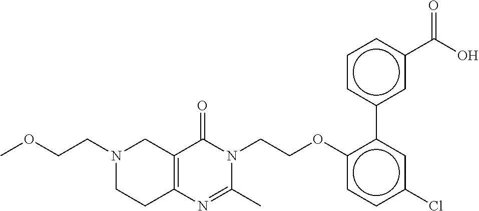 |
| 7 | 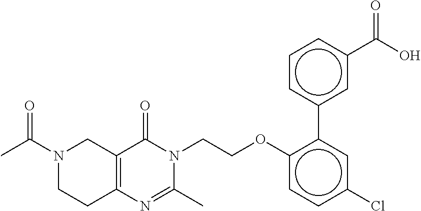 |
| 8 | 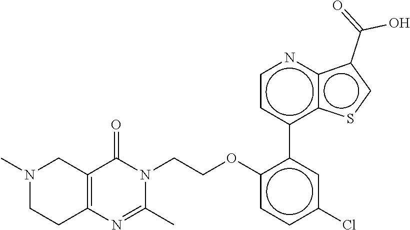 |
| 9 | 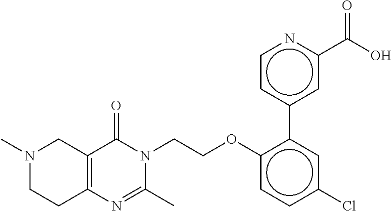 |
| 10 | 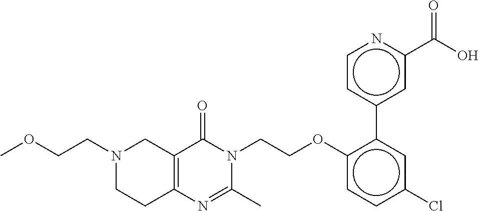 |
| 11 | 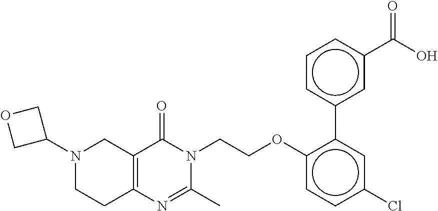 |
| 12 | 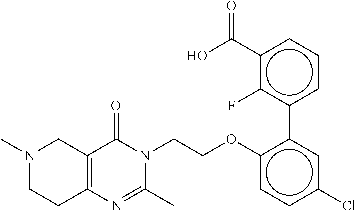 |
| 13 | 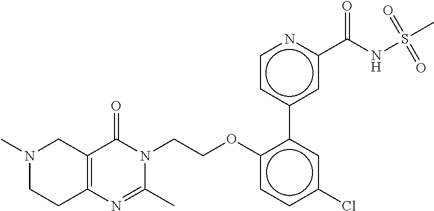 |
| 14 | 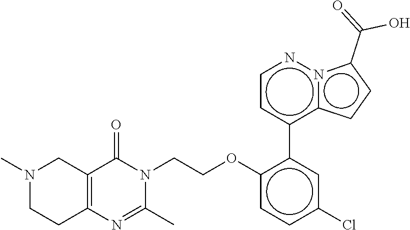 |
| 15 | 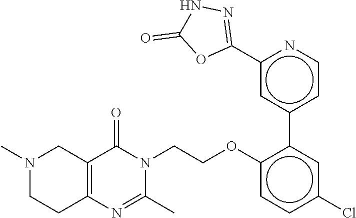 |
| 16 | 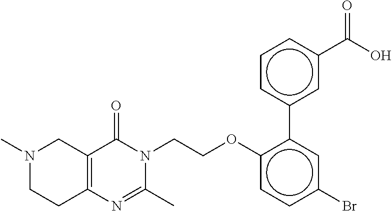 |
| 17 | 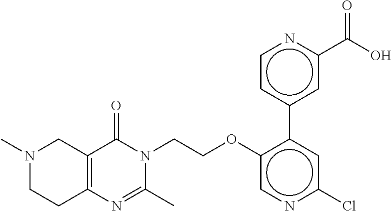 |
| 18 | 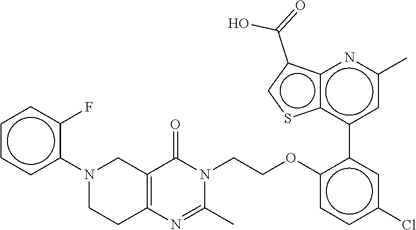 |
| 19 | 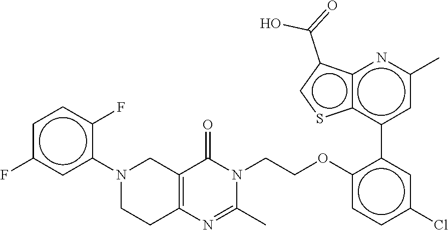 |
| 20 | 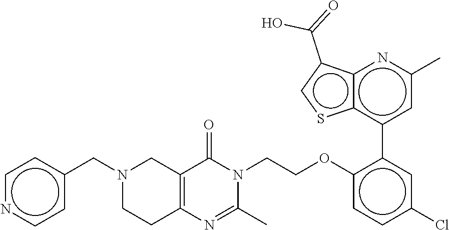 |
| 21 | 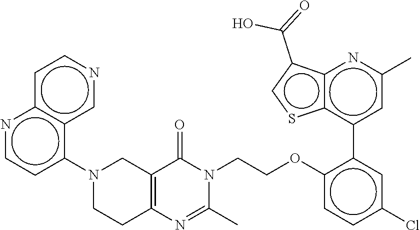 |
| 22 | 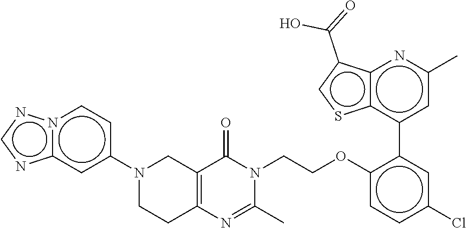 |
| 23 | 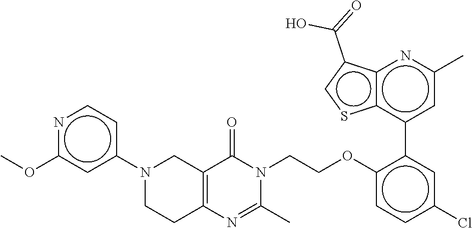 |
| 24 | 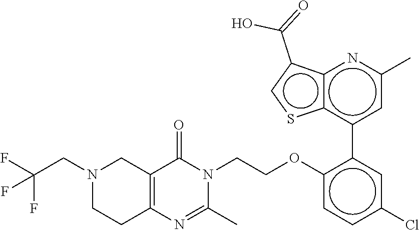 |
| 25 | 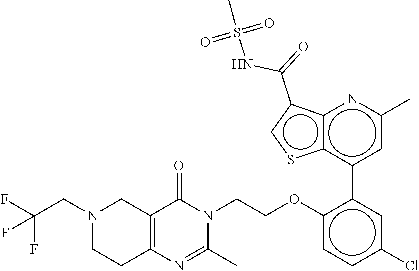 |
| 26 | 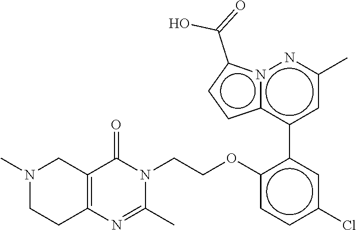 |
| 27 | 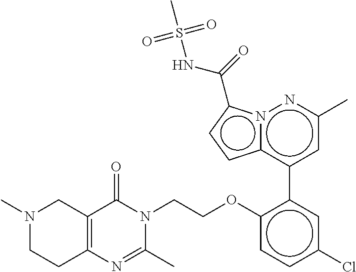 |
| 28 | 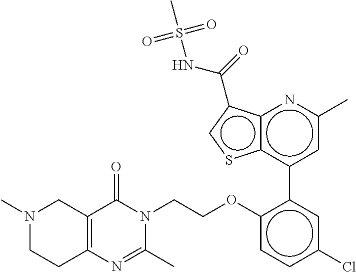 |
| 29 | 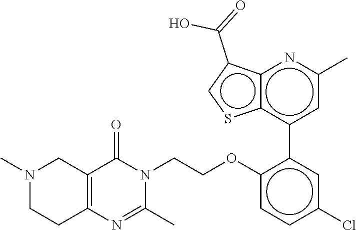 |
| 30 | 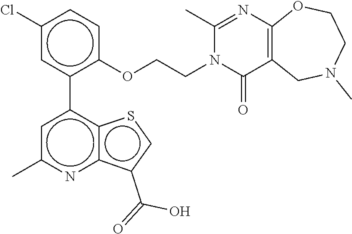 |
| 31 | 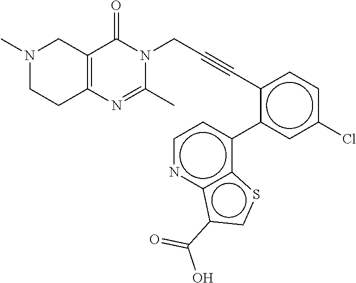 |

The compounds of the present disclosure possess advantageous characteristics, as compared to known compounds, such as known eIF4E inhibitors. For example, the compounds of the present disclosure display more potent eIF4E inhibition activity, more favorable pharmacokinetic properties (e.g., as measured by Cₘₐₓ, Tₘₐₓ, and/or AUC), and/or less interaction with other cellular targets (e.g., hepatic cellular transporter such as OATP1B1) and accordingly improved safety (e.g., drug-drug interaction). These beneficial properties of the compounds of the present disclosure can be measured according to methods commonly available in the art, such as methods exemplified herein.

Due to the existence of double bonds, the compounds of the present disclosure may be in cis or trans, or Z or E, configuration. It is understood that although one configuration may be depicted in the structure of the compounds or formulae of the present disclosure, the present disclosure also encompasses the other configuration. For example, the compounds or formulae of the present disclosure may be depicted in cis or trans, or Z or E, configuration.

In one embodiment, a compound of the present disclosure (e.g., a compound of any of the formulae or any individual compounds disclosed herein) is a pharmaceutically acceptable salt. In another embodiment, a compound of the present disclosure (e.g., a compound of any of the formulae or any individual compounds disclosed herein) is a solvate. In another embodiment, a compound of the present disclosure (e.g., a compound of any of the formulae or any individual compounds disclosed herein) is a hydrate.

### Pharmaceutically Acceptable Salts

In certain embodiments, the compounds disclosed herein exist as their pharmaceutically acceptable salts. In certain embodiments, the methods disclosed herein include methods of treating diseases by administering such pharmaceutically acceptable salts. In certain embodiments, the methods disclosed herein include methods of treating diseases by administering such pharmaceutically acceptable salts as pharmaceutical compositions.

In certain embodiments, the compounds described herein possess acidic or basic groups and therefore react with any of a number of inorganic or organic bases, and inorganic and organic acids, to form a pharmaceutically acceptable salt. In certain embodiments, these salts are prepared in situ during the final isolation and purification of the compounds disclosed herein, or by separately reacting a purified compound in its free form with a suitable acid or base, and isolating the salt thus formed.

Examples of pharmaceutically acceptable salts include those salts prepared by reaction of the compounds described herein with a mineral, organic acid, or inorganic base, such salts including acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxy benzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylateundeconate, and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

In certain embodiments, those compounds described herein which comprise a free acid group react with a suitable base, such as the hydroxide, carbonate, bicarbonate, or sulfate of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine, and the like. It should be understood that the compounds described herein also include the quaternization of any basic nitrogen-containing groups they contain. In certain embodiments, water or oil-soluble or dispersible products are obtained by such quaternization.

### Solvates

“Solvate” refers to forms of the compound that are associated with a solvent or water (also referred to as “hydrate”), usually by a solvolysis reaction. This physical association includes hydrogen bonding. Conventional solvents include water, ethanol, acetic acid and the like. The compounds of the disclosure may be prepared e.g., in crystalline form and may be solvated or hydrated. Suitable solvates include pharmaceutically acceptable solvates, such as hydrates, and further include both stoichiometric solvates and non-stoichiometric solvates. In certain instances, the solvate will be capable of isolation, for example when one or more solvent molecules are incorporated in the crystal lattice of the crystalline solid. “Solvate” encompasses both solution-phase and isolable solvates. Representative solvates include hydrates, ethanolates and methanolates.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates are within the scope of the disclosure.

It will also be appreciated by those skilled in organic chemistry that many organic compounds can exist in more than one crystalline form. For example, crystalline form may vary from solvate to solvate. Thus, all crystalline forms or the pharmaceutically acceptable solvates thereof are contemplated and are within the scope of the present disclosure.

In certain embodiments, the compounds described herein exist as solvates. The present disclosure provides for methods of treating diseases by administering such solvates. The present disclosure further provides for methods of treating diseases by administering such solvates as pharmaceutical compositions.

Solvates contain either stoichiometric or non-stoichiometric amounts of a solvent, such as water, ethanol, and the like. Hydrates are formed when the solvent is water, or alcoholates are formed when the solvent is alcohol. Solvates of the compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

### Isomers (Stereoisomers, Geometric Isomer, Tautomer, Etc.)

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.”

Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+)- or (−)-isomers respectively). A chiral compound can exist as either individual enantiomer or as a mixture thereof. A mixture containing equal proportions of the enantiomers is termed a “racemic mixture”.

As used herein a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. In certain embodiments, the weights are based upon total weight of all enantiomers or stereoisomers of the compound.

As used herein and unless otherwise indicated, the term “enantiomerically pure (R)-compound” refers to at least about 95% by weight (R)-compound and at most about 5% by weight (S)-compound, at least about 99% by weight (R)-compound and at most about 1% by weight (S)-compound, or at least about 99.9% by weight (R)-compound and at most about 0.1% by weight(S)-compound. In certain embodiments, the weights are based upon total weight of compound.

As used herein and unless otherwise indicated, the term “enantiomerically pure(S)-compound” refers to at least about 95% by weight(S)-compound and at most about 5% by weight (R)-compound, at least about 99% by weight(S)-compound and at most about 1% by weight (R)-compound or at least about 99.9% by weight(S)-compound and at most about 0.1% by weight (R)-compound. In certain embodiments, the weights are based upon total weight of compound.

In the compositions provided herein, an enantiomerically pure compound or a pharmaceutically acceptable salt, solvate, hydrate or prodrug thereof can be present with other active or inactive ingredients. For example, a pharmaceutical composition comprising enantiomerically pure (R)-compound can comprise, for example, about 90% excipient and about 10% enantiomerically pure (R)-compound. In certain embodiments, the enantiomerically pure (R)-compound in such compositions can, for example, comprise, at least about 95% by weight (R)-compound and at most about 5% by weight (S)-compound, by total weight of the compound. For example, a pharmaceutical composition comprising enantiomerically pure (S)-compound can comprise, for example, about 90% excipient and about 10% enantiomerically pure (S)-compound. In certain embodiments, the enantiomerically pure (S)-compound in such compositions can, for example, comprise, at least about 95% by weight (S)-compound and at most about 5% by weight (R)-compound, by total weight of the compound. In certain embodiments, the active ingredient can be formulated with little or no excipient or carrier.

Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

In certain embodiments, the compounds described herein exist as geometric isomers. In certain embodiments, the compounds described herein possess one or more double bonds. The compounds disclosed herein include all cis, trans, syn, anti, entgegen (E), and zusammen (Z) isomers as well as the corresponding mixtures thereof. All geometric forms of the compounds disclosed herein are contemplated and are within the scope of the disclosure.

In certain embodiments, the compounds disclosed herein possess one or more chiral centers and each center exists in the R configuration or S configuration. The compounds disclosed herein include all diastereomeric, enantiomeric, and epimeric forms as well as the corresponding mixtures thereof. All diastereomeric, enantiomeric, and epimeric forms of the compounds disclosed herein are contemplated and are within the scope of the disclosure.

In additional embodiments of the compounds and methods provided herein, mixtures of enantiomers and/or diastereoisomers, resulting from a single preparative step, combination, or interconversion are useful for the applications described herein. In certain embodiments, the compounds described herein are prepared as their individual stereoisomers by reacting a racemic mixture of the compound with an optically active resolving agent to form a pair of diastereoisomeric compounds, separating the diastereomers, and recovering the optically pure enantiomers. In certain embodiments, dissociable complexes are preferred. In certain embodiments, the diastereomers have distinct physical properties (e.g., melting points, boiling points, solubilities, reactivity, etc.) and are separated by taking advantage of these dissimilarities. In certain embodiments, the diastereomers are separated by chiral chromatography, or preferably, by separation/resolution techniques based upon differences in solubility. In certain embodiments, the optically pure enantiomer is then recovered, along with the resolving agent.

### Tautomers

In certain embodiments, compounds described herein exist as tautomers. The compounds described herein include all possible tautomers within the formulas described herein.

Tautomers are compounds that are interconvertible by migration of a hydrogen atom, accompanied by a switch of a single bond and an adjacent double bond. In bonding arrangements where tautomerization is possible, a chemical equilibrium of the tautomers will exist. For example, enols and ketones are tautomers because they are rapidly interconverted by treatment with either acid or base. Another example of tautomerism is the aci- and nitro-forms of phenylnitromethane, that are likewise formed by treatment with acid or base. Tautomeric forms may be relevant to the attainment of the optimal chemical reactivity and biological activity of a compound of interest. All tautomeric forms of the compounds disclosed herein are contemplated and are within the scope of the disclosure. The exact ratio of the tautomers depends on several factors, including temperature, solvent, and pH.

### Pharmaceutical Compositions

In certain embodiments, the compound described herein is administered as a pure chemical. In certain embodiments, the compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

Accordingly, the present disclosure provides pharmaceutical compositions comprising a compound described herein, or a pharmaceutically acceptable salt, solvate, stereoisomer, or prodrug thereof, and a pharmaceutically acceptable excipient.

In certain embodiments, the compound provided herein is substantially pure, in that it contains less than about 5%, less than about 1%, or less than about 0.1% of other organic small molecules, such as unreacted intermediates or synthesis by-products that are created, for example, in one or more of the steps of a synthesis method.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

In certain embodiments, the pharmaceutical composition is formulated for oral, topical (including buccal and sublingual), rectal, vaginal, transdermal, parenteral, intrapulmonary, intradermal, intrathecal and epidural and intranasal administration. Parenteral administration includes intramuscular, intravenous, intraarterial, intraperitoneal, or subcutaneous administration. In certain embodiments, the pharmaceutical composition is formulated for intravenous injection, oral administration, inhalation, nasal administration, topical administration, or ophthalmic administration. In certain embodiments, the pharmaceutical composition is formulated for oral administration. In certain embodiments, the pharmaceutical composition is formulated for intravenous injection. In certain embodiments, the pharmaceutical composition is formulated as a tablet, a pill, a capsule, a liquid, an inhalant, a nasal spray solution, a suppository, a suspension, a gel, a colloid, a dispersion, a suspension, a solution, an emulsion, an ointment, a lotion, an eye drop, or an ear drop. In certain embodiments, the pharmaceutical composition is formulated as a tablet.

### Preparation and Characterization of the Compounds

The compounds of the present disclosure can be prepared in a number of ways well known to those skilled in the art of organic synthesis. By way of example, the compounds of the present disclosure can be synthesized using the methods described below, together with synthetic methods known in the art of synthetic organic chemistry, or variations thereon as appreciated by those skilled in the art. The compounds of the present disclosure (i.e., a compound of the present application (e.g., a compound of any of the formulae or any individual compounds disclosed herein)) can be synthesized by following the general synthetic scheme below as well as the steps outlined in the examples, schemes, procedures, and/or synthesis described herein (e.g., Examples).

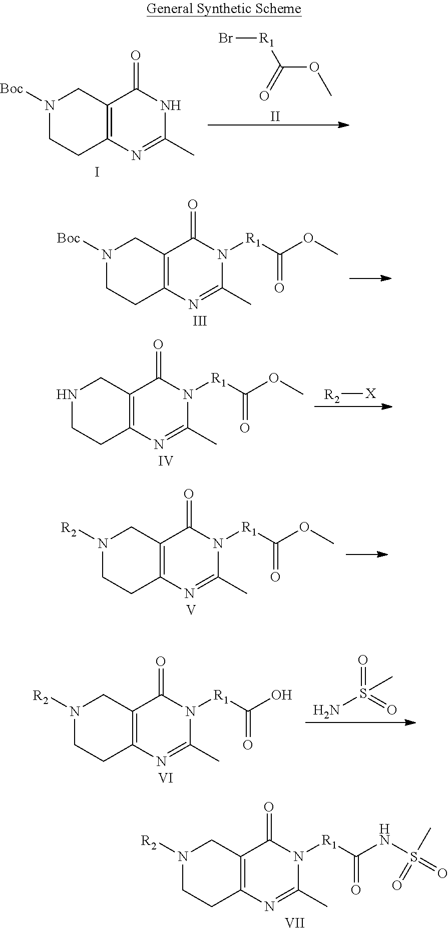

Those skilled in the art will recognize if a stereocenter exists in the compounds of the present disclosure (e.g., a compound of any of the formulae or any individual compounds disclosed herein). Accordingly, the present disclosure includes both possible stereoisomers (unless specified in the synthesis) and includes not only racemic compound but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-Interscience, 1994).

The compounds used in the reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, PA), Aldrich Chemical (Milwaukee, WI, including Sigma Chemical and Fluka) (Pittsburgh, PA).

Suitable reference books and treatises that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Advanced Organic Chemistry: Reactions, Mechanisms and Structure”, 4th Ed., Wiley-Interscience, New York, 1992; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as through on-line. Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

### Analytical Methods, Materials, and Instrumentation

Unless otherwise noted, reagents and solvents were used as received from commercial suppliers. Proton nuclear magnetic resonance (NMR) spectra were obtained on either Bruker or Varian spectrometers at 400 MHz. Spectra are given in ppm (δ) and coupling constants, J, are reported in Hertz. Tetramethylsilane (TMS) was used as an internal standard. Liquid chromatography-mass spectrometry (LC/MS) were collected using a SHIMADZU LCMS-2020EV or Agilent 1260-6125B LCMS. Purity and low resolution mass spectral data were measured using Agilent 1260-6125B LCMS system (with Diode Array Detector, and Agilent G6125BA Mass spectrometer) or using Waters Acquity UPLC system (with Diode Array Detector, and Waters 3100 Mass Detector). The purity was characterized by UV wavelength 214 nm, 220 nm, 254 nm and ESI. Column: poroshell 120 EC-C18 2.7 μm 4.6×100 mm; Flow rate 0.8 mL/min; Solvent A (100/0.1 water/formic acid), Solvent B (100 acetonitrile); gradient: hold 5% B to 0.3 min, 5-95% B from 0.3 to 2 min, hold 95% B to 4.8 min, 95-5% B from 4.8 to 5.4 min, then hold 5% B to 6.5 min. Or, column: Acquity UPLC BEH C18 1.7 μm 2.1×50 mm; Flow rate 0.5 mL/min; Solvent A (0.1% formic acid water), Solvent B (acetonitrile); gradient: hold 5% B for 0.2 min, 5-95% B from 0.2 to 2.0 min, hold 95% B to 3.1 min, then 5% B at 3.5 min.

### Biological Assays

The biological activities of the compounds of the present application can be assessed with methods and assays known in the art.

For example, the affinity of compounds for proteins can be determined via a variety of biophysical assay formats, including surface plasmon resonance (SPR), isothermal titration calorimetry (ITC), mass spectrometry-based binding methods, and others. The competitive binding activity and potency of compounds for proteins can be assessed by a range of routine biochemical methods, including fluorescence polarization (FP), time-resolved fluorescence energy transfer (TR-FRET), and others. The functional effects of compounds on complex biological systems can be assessed in cell-free or cell lysate based assays such as in vitro translation, and others. The effects of compounds on specific target protein-related functions in cells (cellular activity and potency) can be assessed by a wide range of different methods, including reporter assays, immunoassays such as Western blot, cellular enzyme-linked immunoassays (ELISA), high-content imaging, and others. The effects of compounds on cellular phenotypes can be assayed by a range of methods, including those that measure cell proliferation, cell cycle progression, cell viability, cell death, cell migration, cell invasion, cell metabolism, and other cellular phenotypes.

### Methods of Use

In certain aspects, the present disclosure provides methods of inhibiting a protein in a subject or biological sample comprising administering the compound disclosed herein to the subject or contacting the biological sample with the compound disclosed herein.

In certain aspects, the present disclosure provides uses of the compound disclosed herein in the manufacture of a medicament for inhibiting a protein in a subject or biological sample.

In certain aspects, the present disclosure provides compounds disclosed herein for use in inhibiting a protein in a subject or biological sample.

In certain embodiments, the protein is eIF4E.

In certain aspects, the present disclosure provides methods of treating or preventing a disease or disorder a subject in need thereof, comprising administering to the subject the compound disclosed herein.

In certain aspects, the present disclosure provides uses of the compound disclosed herein in the manufacture of a medicament for treating or preventing a disease or disorder in a subject in need thereof.

In certain aspects, the present disclosure provides compounds disclosed herein for use in treating or preventing a disease or disorder in a subject in need thereof.

In certain embodiments, the disease or disorder is an eIF4E-mediated disease or disorder.

In certain embodiments, the disease or disorder is cancer.

In certain embodiments, the cancer includes, but is not limited to, one or more of the cancers of Table A.

TABLE A

| | | | |
|---|---|---|---|
| adrenal cancer | acinic cell carcinoma | acoustic neuroma | acral lentigious |
| | | | melanoma |
| acrospiroma | acute eosinophilic | acute erythroid | acute lymphoblastic |
| | leukemia | leukemia | leukemia |
| acute | acute monocytic | acute promyelocytic | adenocarcinoma |
| megakaryoblastic | leukemia | leukemia | |
| leukemia | | | |
| adenoid cystic | adenoma | adenomatoid | adenosquamous |
| carcinoma | | odontogenic tumor | carcinoma |
| adipose tissue | adrenocortical | adult T-cell | aggressive NK-cell |
| neoplasm | carcinoma | leukemia/lymphoma | leukemia |
| AIDS-related | alveolar | alveolar soft part | ameloblastic |
| lymphoma | rhabdomyosarcoma | sarcoma | fibroma |
| anaplastic large cell | anaplastic thyroid | angioimmunoblastic | angiomyolipoma |
| lymphoma | cancer | T-cell lymphoma | |
| angiosarcoma | astrocytoma | atypical teratoid | B-cell chronic |
| | | rhabdoid tumor | lymphocytic |
| | | | leukemia |
| B-cell | B-cell lymphoma | basal cell carcinoma | biliary tract cancer |
| prolymphocytic | | | |
| leukemia | | | |
| bladder cancer | blastoma | bone cancer | Brenner tumor |
| Brown tumor | Burkitt's lymphoma | breast cancer | brain cancer |
| carcinoma | carcinoma in situ | carcinosarcoma | cartilage tumor |
| cementoma | myeloid sarcoma | chondroma | chordoma |
| choriocarcinoma | choroid plexus | clear-cell sarcoma of | craniopharyngioma |
| | papilloma | the kidney | |
| cutaneous T-cell | cervical cancer | colorectal cancer | Degos disease |
| lymphoma | | | |
| desmoplastic small | diffuse large B-cell | dysembryoplastic | dysgerminoma |
| round cell tumor | lymphoma | neuroepithelial | |
| | | tumor | |
| embryonal | endocrine gland | endodermal sinus | enteropathy- |
| carcinoma | neoplasm | tumor | associated T-cell |
| | | | lymphoma |
| esophageal cancer | fetus in fetu | fibroma | fibrosarcoma |
| follicular lymphoma | follicular thyroid | ganglioneuroma | gastrointestinal |
| | cancer | | cancer |
| germ cell tumor | gestational | giant cell | giant cell tumor of |
| | choriocarcinoma | fibroblastoma | the bone |
| glial tumor | glioblastoma | glioma | gliomatosis cerebri |
| | multiforme | | |
| glucagonoma | gonadoblastoma | granulosa cell tumor | gynandroblastoma |
| gallbladder cancer | gastric cancer | hairy cell leukemia | hemangioblastoma |
| head and neck | hemangiopericytoma | hematological cancer | hepatoblastoma |
| cancer | | | |
| hepatosplenic T-cell | Hodgkin's lymphoma | non-Hodgkin's | invasive lobular |
| lymphoma | | lymphoma | carcinoma |
| intestinal cancer | kidney cancer | laryngeal cancer | lentigo maligna |
| lethal midline | leukemia | leydig cell tumor | liposarcoma |
| carcinoma | | | |
| lung cancer | lymphangioma | lymphangiosarcoma | lymphoepithelioma |
| lymphoma | acute lymphocytic | acute myelogeous | chronic lymphocytic |
| | leukemia | leukemia | leukemia |
| liver cancer | small cell lung | non-small cell lung | MALT lymphoma |
| | cancer | cancer | |
| malignant fibrous | malignant peripheral | malignant triton | mantle cell |
| histiocytoma | nerve sheath tumor | tumor | lymphoma |
| marginal zone B- | mast cell leukemia | mediastinal germ | medullary |
| cell lymphoma | | cell tumor | carcinoma of the |
| | | | breast |
| medullary thyroid | medulloblastoma | melanoma | meningioma |
| cancer | | | |
| merkel cell cancer | mesothelioma | metastatic urothelial | mixed Mullerian |
| | | carcinoma | tumor |
| mucinous tumor | multiple myeloma | muscle tissue | mycosis fungoides |
| | | neoplasm | |
| myxoid liposarcoma | myxoma | myxosarcoma | nasopharyngeal |
| | | | carcinoma |
| neurinoma | neuroblastoma | neurofibroma | neuroma |
| nodular melanoma | ocular cancer | oligoastrocytoma | oligodendroglioma |
| oncocytoma | optic nerve sheath | optic nerve tumor | oral cancer |
| | meningioma | | |
| osteosarcoma | ovarian cancer | Pancoast tumor | papillary thyroid |
| | | | cancer |
| paraganglioma | pinealoblastoma | pineocytoma | pituicytoma |
| pituitary adenoma | pituitary tumor | plasmacytoma | polyembryoma |
| precursor T- | primary central | primary effusion | preimary peritoneal |
| lymphoblastic | nervous system | lymphoma | cancer |
| lymphoma | lymphoma | | |
| prostate cancer | pancreatic cancer | pharyngeal cancer | pseudomyxoma |
| | | | periotonei |
| renal cell carcinoma | renal medullary | retinoblastoma | rhabdomyoma |
| | carcinoma | | |
| rhabdomyosarcoma | Richter's | rectal cancer | sarcoma |
| | transformation | | |
| Schwannomatosis | seminoma | Sertoli cell tumor | sex cord-gonadal |
| | | | stromal tumor |
| signet ring cell | skin cancer | small blue round cell | small cell carcinoma |
| carcinoma | | tumors | |
| soft tissue sarcoma | somatostatinoma | soot wart | spinal tumor |
| splenic marginal | squamous cell | synovial sarcoma | Sezary's disease |
| zone lymphoma | carcinoma | | |
| small intestine | squamous carcinoma | stomach cancer | T-cell lymphoma |
| cancer | | | |
| testicular cancer | thecoma | thyroid cancer | transitional cell |
| | | | carcinoma |
| throat cancer | urachal cancer | urogenital cancer | urothelial carcinoma |
| uveal melanoma | uterine cancer | verrucous carcinoma | visual pathway |
| | | | glioma |
| vulvar cancer | vaginal cancer | Waldenstrom's | Warthin's tumor |
| | | macroglobulinemia | |
| Wilms' tumor | | | |

In certain embodiments, the cancer is a solid tumor. In certain embodiments, the cancer a hematological cancer. Exemplary hematological cancers include, but are not limited to, the cancers listed in Table B. In certain embodiments, the hematological cancer is acute lymphocytic leukemia, chronic lymphocytic leukemia (including B-cell chronic lymphocytic leukemia), or acute myeloid leukemia.

TABLE B

| | |
|---|---|
| acute lymphocytic leukemia (ALL) | acute eosinophilic leukemia |
| acute myeloid leukemia (AML) | acute erythroid leukemia |
| chronic lymphocytic leukemia (CLL) | acute lymphoblastic leukemia |
| small lymphocytic lymphoma (SLL) | acute megakaryoblastic leukemia |
| multiple myeloma (MM) | acute monocytic leukemia |
| Hodgkins lymphoma (HL) | acute promyelocytic leukemia |
| non-Hodgkin's lymphoma (NHL) | acute myelogeous leukemia |
| mantle cell lymphoma (MCL) | B-cell prolymphocytic leukemia |
| marginal zone B-cell lymphoma | B-cell lymphoma |
| splenic marginal zone lymphoma | MALT lymphoma |
| follicular lymphoma (FL) | precursor T-lymphoblastic lymphoma |
| Waldenstrom's macroglobulinemia (WM) | T-cell lymphoma |
| diffuse large B-cell lymphoma (DLBCL) | mast cell leukemia |
| marginal zone lymphoma (MZL) | adult T cell leukemia/lymphoma |
| hairy cell leukemia (HCL) | aggressive NK-cell leukemia |
| Burkitt's lymphoma (BL) | angioimmunoblastic T-cell lymphoma |
| Richter's transformation | |

In certain embodiments, the cancer is colon cancer, gastric cancer, thyroid cancer, lung cancer, leukemia, B-cell lymphoma, T-cell lymphoma, hairy cell lymphoma, Hodgkin's lymphoma, non-Hodgkin's lymphoma, Burkitt's lymphoma, pancreatic cancer, melanoma, multiple melanoma, brain cancer, CNS cancer, renal cancer, prostate cancer, ovarian cancer, breast cancer, liver cancer, mesothelioma, rectal cancer, esophageal cancer, head and neck cancers, pancreatic cancer, uterine cancer, cervical cancer, or bladder cancer.

In certain embodiments, the disease or disorder is a non-cancer disease or disorder (e.g., an eIF4E-mediated non-cancer disease or disorder). In certain embodiments, the disease or disorder is cytokine related diseases, such as inflammatory diseases, allergies, or other conditions associated with proinflammatory cytokines. In certain embodiments, the disease or disorder is fibrotic diseases. In certain embodiments, the disease or disorder is a disease or disorder associated with the expression (or aberrant expression) and/or function (or dysfunction) of the eIF4E or in which the expression (or aberrant expression) and/or function (or dysfunction) of the elF4E plays a role (e.g., in the initiation and/or development).

In certain embodiments, the subject is a mammal.

In certain embodiments, the subject is a human.

### Definitions

As used in the specification and appended claims, unless specified to the contrary, the following terms have the meaning indicated below.

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5th Edition, John Wiley &amp; Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPFC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. F. Eliel, Ed., Univ. of Notre Dame Press, Notre Dame, IN 1972).

The disclosure additionally encompasses compounds described herein as individual isomers substantially free of other isomers, and alternatively, as mixtures of various isomers.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The following terms are intended to have the meanings presented therewith below and are useful in understanding the description and intended scope of the present disclosure. When describing the disclosure, which may include compounds, pharmaceutical compositions containing such compounds and methods of using such compounds and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein. The articles “a” and “an” may be used herein to refer to one or to more than one (i.e., at least one) of the grammatical objects of the article. By way of example “an analogue” means one analogue or more than one analogue.

“Alkyl” as used herein, refers to a radical of a straight-chain or branched saturated hydrocarbon group having from 1 to 20 carbon atoms (“C₁₋₂₀ alkyl”). In certain embodiments, an alkyl group has 1 to 12 carbon atoms (“C₁₋₁₂ alkyl”). In certain embodiments, an alkyl group has 1 to 10 carbon atoms (“C₁₋₁₀ alkyl”). In certain embodiments, an alkyl group has 1 to 9 carbon atoms (“C₁₋₉ alkyl”). In certain embodiments, an alkyl group has 1 to 8 carbon atoms (“C₁₋₈ alkyl”). In certain embodiments, an alkyl group has 1 to 7 carbon atoms (“C₁₋₇ alkyl”). In certain embodiments, an alkyl group has 1 to 6 carbon atoms (“C₁₋₆ alkyl”, which is also referred to herein as “lower alkyl”). In certain embodiments, an alkyl group has 1 to 5 carbon atoms (“C₁₋₅ alkyl”). In certain embodiments, an alkyl group has 1 to 4 carbon atoms (“C₁₋₄ alkyl”). In certain embodiments, an alkyl group has 1 to 3 carbon atoms (“C₁₋₃ alkyl”). In certain embodiments, an alkyl group has 1 to 2 carbon atoms (“C₁₋₂ alkyl”). In certain embodiments, an alkyl group has 1 carbon atom (“C₁ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), isobutyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁₋₁₀ alkyl (e.g., —CH₃). In certain embodiments, the alkyl group is substituted C₁₋₁₀ alkyl. Common alkyl abbreviations include Me (—CH₃), Et (—CH₂CH₃), i-Pr (—CH(CH₃)₂), n-Pr (—CH₂CH₂CH₃), n-Bu (—CH₂CH₂CH₂CH₃), or i-Bu (—CH₂CH(CH₃)₂).

“Alkylene” as used herein, refers to an alkyl group wherein two hydrogens are removed to provide a divalent radical. When a range or number of carbons is provided for a particular “alkylene” group, it is understood that the range or number refers to the range or number of carbons in the linear carbon divalent chain. An “alkelene” group may be substituted or unsubstituted with one or more substituents as described herein. Exemplary unsubstituted divalent alkylene groups include, but are not limited to, methylene (—CH₂—), ethylene (—CH₂CH₂—), propylene (—CH₂CH₂CH₂—), butylene (—CH₂CH₂CH₂CH₂—), pentylene (—CH₂CH₂CH₂CH₂CH₂—), hexylene-CH₂CH₂CH₂CH₂CH₂CH₂—), and the like. Exemplary substituted divalent alkylene groups, e.g., substituted with one or more alkyl (methyl) groups, include but are not limited to, substituted methylene (—CH(CH₃)—, (—C(CH₃)₂—), substituted ethylene (—CH(CH₃)CH₂—, —CH₂CH(CH₃)—, —C(CH₃)₂CH₂—, —CH₂C(CH₃)₂—), substituted propylene (—CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—), and the like.

“Alkenyl” as used herein, refers to a radical of a straight-chain or branched hydrocarbon group having from 2 to 20 carbon atoms, one or more carbon-carbon double bonds (e.g., 1, 2, 3, or 4 carbon-carbon double bonds), and optionally one or more carbon-carbon triple bonds (e.g., 1, 2, 3, or 4 carbon-carbon triple bonds) (“C₂₋₂₀ alkenyl”). In certain embodiments, alkenyl does not contain any triple bonds. In certain embodiments, an alkenyl group has 2 to 10 carbon atoms (“C₂₋₁₀ alkenyl”). In certain embodiments, an alkenyl group has 2 to 9 carbon atoms (“C₂₋₉ alkenyl”). In certain embodiments, an alkenyl group has 2 to 8 carbon atoms (“C₂₋₈ alkenyl”). In certain embodiments, an alkenyl group has 2 to 7 carbon atoms (“C₂₋₇ alkenyl”). In certain embodiments, an alkenyl group has 2 to 6 carbon atoms (“C₂₋₆ alkenyl”). In certain embodiments, an alkenyl group has 2 to 5 carbon atoms (“C₂₋₅ alkenyl”). In certain embodiments, an alkenyl group has 2 to 4 carbon atoms (“C₂₋₄ alkenyl”). In certain embodiments, an alkenyl group has 2 to 3 carbon atoms (“C₂₋₃ alkenyl”). In certain embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₅), and the like. Unless otherwise specified, each instance of an alkenyl group is independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₂₋₁₀ alkenyl. In certain embodiments, the alkenyl group is substituted C₂₋₁₀ alkenyl.

“Alkenylene” as used herein, refers to an alkenyl group wherein two hydrogens are removed to provide a divalent radical. When a range or number of carbons is provided for a particular “alkenylene” group, it is understood that the range or number refers to the range or number of carbons in the linear carbon divalent chain. An “alkenylene” group may be substituted or unsubstituted with one or more substituents as described herein. Exemplary unsubstituted divalent alkenylene groups include, but are not limited to, ethenylene (—CH═CH—) and propenylene (e.g., —CH═CHCH₂—, —CH₂—CH═CH—). Exemplary substituted divalent alkenylene groups, e.g., substituted with one or more alkyl (methyl) groups, include but are not limited to, substituted ethylene (—C(CH₃)—CH—, —CH═C(CH₃)—), substituted propylene (e.g., —C(CH₃)—CHCH₂—, —CH═C(CH₃)CH₂—, —CH═CHCH(CH₃)—, —CH═CHC(CH₃)₂—, —CH(CH₃)—CH═CH—, —C(CH₃)₂—CH═CH—, —CH₂—C(CH₃)═CH—, —CH₂—CH═C(CH₃)—), and the like.

“Alkynyl” as used herein, refers to a radical of a straight-chain or branched hydrocarbon group having from 2 to 20 carbon atoms, one or more carbon-carbon triple bonds (e.g., 1, 2, 3, or 4 carbon-carbon triple bonds), and optionally one or more carbon-carbon double bonds (e.g., 1, 2, 3, or 4 carbon-carbon double bonds) (“C₂₋₂₀ alkynyl”). In certain embodiments, alkynyl does not contain any double bonds. In certain embodiments, an alkynyl group has 2 to 10 carbon atoms (“C₂₋₁₀ alkynyl”). In certain embodiments, an alkynyl group has 2 to 9 carbon atoms (“C₂₋₉ alkynyl”). In certain embodiments, an alkynyl group has 2 to 8 carbon atoms (“C₂₋₈ alkynyl”). In certain embodiments, an alkynyl group has 2 to 7 carbon atoms (“C₂₋₇ alkynyl”). In certain embodiments, an alkynyl group has 2 to 6 carbon atoms (“C₂₋₆ alkynyl”). In certain embodiments, an alkynyl group has 2 to 5 carbon atoms (“C₂₋₅ alkynyl”). In certain embodiments, an alkynyl group has 2 to 4 carbon atoms (“C₂₋₄ alkynyl”). In certain embodiments, an alkynyl group has 2 to 3 carbon atoms (“C₂₋₃ alkynyl”). In certain embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently optionally substituted, i.e., unsubstituted (an “unsubstituted alkynyl”) or substituted (a “substituted alkynyl”) with one or more substituents; e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂₋₁₀ alkynyl.

“Alkynylene” as used herein, refers to a linear alkynyl group wherein two hydrogens are removed to provide a divalent radical. When a range or number of carbons is provided for a particular “alkynylene” group, it is understood that the range or number refers to the range or number of carbons in the linear carbon divalent chain. An “alkynylene” group may be substituted or unsubstituted with one or more substituents as described herein. Exemplary divalent alkynylene groups include, but are not limited to, substituted or unsubstituted ethynylene, substituted or unsubstituted propynylene, and the like.

The term “heteroalkyl,” as used herein, refers to an alkyl group, as defined herein, which further comprises 1 or more (e.g., 1, 2, 3, or 4) heteroatoms (e.g., oxygen, sulfur, nitrogen, boron, silicon, phosphorus) within the parent chain, wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkyl group refers to a saturated group having from 1 to 10 carbon atoms and 1, 2, 3, or 4 heteroatoms (“C₁₋₁₀ heteroalkyl”). In certain embodiments, a heteroalkyl group is a saturated group having 1 to 9 carbon atoms and 1, 2, 3, or 4 heteroatoms (“C₁₋₉ heteroalkyl”). In certain embodiments, a heteroalkyl group is a saturated group having 1 to 8 carbon atoms and 1, 2, 3, or 4 heteroatoms (“C₁₋₈ heteroalkyl”). In certain embodiments, a heteroalkyl group is a saturated group having 1 to 7 carbon atoms and 1, 2, 3, or 4 heteroatoms (“C₁₋₇ heteroalkyl”). In certain embodiments, a heteroalkyl group is a group having 1 to 6 carbon atoms and 1, 2, or 3 heteroatoms (“C₁₋₆ heteroalkyl”). In certain embodiments, a heteroalkyl group is a saturated group having 1 to 5 carbon atoms and 1 or 2 heteroatoms (“C₁₋₅ heteroalkyl”). In certain embodiments, a heteroalkyl group is a saturated group having 1 to 4 carbon atoms and/or 2 heteroatoms (“C₁₋₄ heteroalkyl”). In certain embodiments, a heteroalkyl group is a saturated group having 1 to 3 carbon atoms and 1 heteroatom (“C₁₋₃ heteroalkyl”). In certain embodiments, a heteroalkyl group is a saturated group having 1 to 2 carbon atoms and 1 heteroatom (“C₁₋₂ heteroalkyl”). In certain embodiments, a heteroalkyl group is a saturated group having 1 carbon atom and 1 heteroatom (“C₁ heteroalkyl”). In certain embodiments, a heteroalkyl group is a saturated group having 2 to 6 carbon atoms and 1 or 2 heteroatoms (“C₂₋₆ heteroalkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted C₁₋₁₀ heteroalkyl. In certain embodiments, the heteroalkyl group is a substituted C₁₋₁₀ heteroalkyl.

The term “heteroalkenyl,” as used herein, refers to an alkenyl group, as defined herein, which further comprises one or more (e.g., 1, 2, 3, or 4) heteroatoms (e.g., oxygen, sulfur, nitrogen, boron, silicon, phosphorus) wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkenyl group refers to a group having from 2 to 10 carbon atoms, at least one double bond, and 1, 2, 3, or 4 heteroatoms (“C₂₋₁₀ heteroalkenyl”). In certain embodiments, a heteroalkenyl group has 2 to 9 carbon atoms at least one double bond, and 1, 2, 3, or 4 heteroatoms (“C₂₋₉ heteroalkenyl”). In certain embodiments, a heteroalkenyl group has 2 to 8 carbon atoms, at least one double bond, and 1, 2, 3, or 4 heteroatoms (“C₂₋₈ heteroalkenyl”). In certain embodiments, a heteroalkenyl group has 2 to 7 carbon atoms, at least one double bond, and 1, 2, 3, or 4 heteroatoms (“C₂₋₇ heteroalkenyl”). In certain embodiments, a heteroalkenyl group has 2 to 6 carbon atoms, at least one double bond, and 1, 2, or 3 heteroatoms (“C₂₋₆ heteroalkenyl”). In certain embodiments, a heteroalkenyl group has 2 to 5 carbon atoms, at least one double bond, and 1 or 2 heteroatoms (“C₂₋₅ heteroalkenyl”). In certain embodiments, a heteroalkenyl group has 2 to 4 carbon atoms, at least one double bond, and 1 or 2 heteroatoms (“C₂₋₄ heteroalkenyl”). In certain embodiments, a heteroalkenyl group has 2 to 3 carbon atoms, at least one double bond, and 1 heteroatom (“C₂₋₃ heteroalkenyl”). In certain embodiments, a heteroalkenyl group has 2 to 6 carbon atoms, at least one double bond, and 1 or 2 heteroatoms (“C₂₋₆ heteroalkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted C₂₋₁₀ heteroalkenyl. In certain embodiments, the heteroalkenyl group is a substituted C₂₋₁₀ heteroalkenyl.

The term “heteroalkynyl,” as used herein, refers to an alkynyl group, as defined herein, which further comprises one or more (e.g., 1, 2, 3, or 4) heteroatoms (e.g., oxygen, sulfur, nitrogen, boron, silicon, phosphorus) wherein the one or more heteroatoms is inserted between adjacent carbon atoms within the parent carbon chain and/or one or more heteroatoms are inserted between a carbon atom and the parent molecule, i.e., between the point of attachment. In certain embodiments, a heteroalkynyl group refers to a group having from 2 to 10 carbon atoms, at least one triple bond, and 1, 2, 3, or 4 heteroatoms (“C₂₋₁₀ heteroalkynyl”). In certain embodiments, a heteroalkynyl group has 2 to 9 carbon atoms, at least one triple bond, and 1, 2, 3, or 4 heteroatoms (“C₂₋₉ heteroalkynyl”). In certain embodiments, a heteroalkynyl group has 2 to 8 carbon atoms, at least one triple bond, and 1, 2, 3, or 4 heteroatoms (“C₂₋₈ heteroalkynyl”). In certain embodiments, a heteroalkynyl group has 2 to 7 carbon atoms, at least one triple bond, and 1, 2, 3, or 4 heteroatoms (“C₂₋₇ heteroalkynyl”). In certain embodiments, a heteroalkynyl group has 2 to 6 carbon atoms, at least one triple bond, and 1, 2, or 3 heteroatoms (“C₂₋₆ heteroalkynyl”). In certain embodiments, a heteroalkynyl group has 2 to 5 carbon atoms, at least one triple bond, and 1 or 2 heteroatoms (“C₂₋₅ heteroalkynyl”). In certain embodiments, a heteroalkynyl group has 2 to 4 carbon atoms, at least one triple bond, and lor 2 heteroatoms (“C₂₋₄ heteroalkynyl”). In certain embodiments, a heteroalkynyl group has 2 to 3 carbon atoms, at least one triple bond, and 1 heteroatom (“C₂₋₃ heteroalkynyl”). In certain embodiments, a heteroalkynyl group has 2 to 6 carbon atoms, at least one triple bond, and 1 or 2 heteroatoms (“C₂₋₆ heteroalkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted C₂₋₁₀ heteroalkynyl. In certain embodiments, the heteroalkynyl group is a substituted C₂₋₁₀ heteroalkynyl.

Analogous to “alkylene,” “alkenylene,” and “alkynylene” as defined above, “heteroalkylene,” “heteroalkenylene,” and “heteroalkynylene,” as used herein, refer to a divalent radical of heteroalkyl, heteroalkenyl, and heteroalkynyl group respectively. When a range or number of carbons is provided for a particular “heteroalkylene,” “heteroalkenylene,” or “heteroalkynylene,” group, it is understood that the range or number refers to the range or number of carbons in the linear divalent chain. “Heteroalkylene,” “heteroalkenylene,” and “heteroalkynylene” groups may be substituted or unsubstituted with one or more substituents as described herein.

“Aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In certain embodiments, an aryl group has six ring carbon atoms (“C₆ aryl”; e.g., phenyl). In certain embodiments, an aryl group has ten ring carbon atoms (“C₁₀ aryl”; e.g., naphthyl such as 1-naphthyl and 2-naphthyl). In certain embodiments, an aryl group has fourteen ring carbon atoms (“C₁₄ aryl”; e.g., anthracyl).

Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, and trinaphthalene. Particular aryl groups include phenyl, naphthyl, indenyl, and tetrahydronaphthyl. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

“Fused aryl” refers to an aryl having two of its ring carbon in common with a second aryl or heteroaryl ring or with a carbocyclyl or heterocyclyl ring.

“Aralkyl” is a subset of alkyl and aryl, as defined herein, and refers to an optionally substituted alkyl group substituted by an optionally substituted aryl group.

“Heteroaryl” refers to a radical of a 5- to 14-membered monocyclic or polycyclic 4n+2 aromatic ring system (e.g., having 6, 10, or 14 π electrons shared in a cyclic array) having ring carbon atoms and 1-8 ring heteroatoms provided in the aromatic ring system, wherein each heteroatom is independently selected from nitrogen, oxygen and sulfur (“5- to 14-membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings.

“Heteroaryl” also includes ring systems wherein the heteroaryl ring, as defined above, is fused with one or more carbocyclyl or heterocyclyl groups wherein the point of attachment is on the heteroaryl ring, and in such instances, the number of ring members continues to designate the number of ring members in the heteroaryl ring system. When substitution is indicated in such instances, unless otherwise specified, substitution can occur on either the heteroaryl or the one or more carbocyclyl or heterocyclyl groups. “Heteroaryl” also includes ring systems wherein the heteroaryl ring, as defined above, is fused with one or more aryl groups wherein the point of attachment is either on the aryl or heteroaryl ring, and in such instances, the number of ring members designates the total number of ring members in the fused (aryl/heteroaryl) ring system. When substitution is indicated in such instances, unless otherwise specified, substitution can occur on either the heteroaryl or the one or more aryl groups. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In certain embodiments, a heteroaryl is a 5- to 10-membered aromatic ring system having ring carbon atoms and 1-4 ring heteroatoms provided in the aromatic ring system, wherein each heteroatom is independently selected from nitrogen, oxygen, and sulfur (“5- to 10-membered heteroaryl”). In certain embodiments, a heteroaryl is a 5- to 9-membered aromatic ring system having ring carbon atoms and 1-4 ring heteroatoms provided in the aromatic ring system, wherein each heteroatom is independently selected from nitrogen, oxygen, and sulfur (“5- to 9-membered heteroaryl”). In certain embodiments, a heteroaryl is a 5- to 8-membered aromatic ring system having ring carbon atoms and 1-4 ring heteroatoms provided in the aromatic ring system, wherein each heteroatom is independently selected from nitrogen, oxygen, and sulfur (“5- to 8-membered heteroaryl”). In certain embodiments, a heteroaryl group is a 5- to 6-membered aromatic ring system having ring carbon atoms and 1-4 ring heteroatoms provided in the aromatic ring system, wherein each heteroatom is independently selected from nitrogen, oxygen, and sulfur (“5- to 6-membered heteroaryl”). In certain embodiments, the 5- to 6-membered heteroaryl has 1-3 ring heteroatoms independently selected from nitrogen, oxygen, and sulfur. In certain embodiments, the 5- to 6-membered heteroaryl has 1-2 ring heteroatoms independently selected from nitrogen, oxygen, and sulfur. In certain embodiments, the 5- to 6-membered heteroaryl has 1 ring heteroatom selected from nitrogen, oxygen, and sulfur. Unless otherwise specified, each instance of a heteroaryl group is independently optionally substituted, i.e., unsubstituted (an “unsubstituted heteroaryl”) or substituted (a “substituted heteroaryl”) with one or more substituents. In certain embodiments, the heteroaryl group is unsubstituted 5- to 14-membered heteroaryl. In certain embodiments, the heteroaryl group is substituted 5- to 14-membered heteroaryl.

Exemplary 5-membered heteroaryl containing one heteroatom include, without limitation, pyrrolyl, furanyl and thiophenyl. Exemplary 5-membered heteroaryl containing two heteroatoms include, without limitation, imidazolyl, pyrazolyl, oxazolyl, isoxazolyl, thiazolyl, and isothiazolyl. Exemplary 5-membered heteroaryl containing three heteroatoms include, without limitation, triazolyl, oxadiazolyl, and thiadiazolyl. Exemplary 5-membered heteroaryl containing four heteroatoms include, without limitation, tetrazolyl. Exemplary 6-membered heteroaryl containing one heteroatom include, without limitation, pyridinyl. Exemplary 6-membered heteroaryl containing two heteroatoms include, without limitation, pyridazinyl, pyrimidinyl, and pyrazinyl. Exemplary 6-membered heteroaryl containing three or four heteroatoms include, without limitation, triazinyl and tetrazinyl, respectively. Exemplary 7-membered heteroaryl containing one heteroatom include, without limitation, azepinyl, oxepinyl, and thiepinyl. Exemplary 5,6-bicyclic heteroaryl include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl include, without limitation, naphthyridinyl, pteridinyl, quinolinyl, isoquinolinyl, cinnolinyl, quinoxalinyl, phthalazinyl, and quinazolinyl.

“Heteroaralkyl” is a subset of alkyl and heteroaryl, as defined herein, and refers to an optionally substituted alkyl group substituted by an optionally substituted heteroaryl group.

“Carbocyclyl” refers to a radical of a non-aromatic cyclic hydrocarbon group having from 3 to 12 ring carbon atoms (“C₃₋₁₂ carbocyclyl”) and zero heteroatoms in the nonaromatic ring system. In certain embodiments, a carbocyclyl group has 3 to 10 ring carbon atoms (“C₃₋₁₀ carbocyclyl”). In certain embodiments, a carbocyclyl group has 3 to 8 ring carbon atoms (“C₃₋₈ carbocyclyl”). In certain embodiments, a carbocyclyl group has 3 to 6 ring carbon atoms (“C₃₋₆ carbocyclyl”). In certain embodiments, a carbocyclyl group has 5 to 6 ring carbon atoms (“C₅₋₆ carbocyclyl”). In certain embodiments, a carbocyclyl group has 5 to 10 ring carbon atoms (“C₅₋₁₀ carbocyclyl”). Exemplary C₃₋₆ carbocyclyl include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₅), and the like. Exemplary C₃₋₁₀ carbocyclyl include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like.

In certain embodiments, “carbocyclyl” is a monocyclic, saturated carbocyclyl group having from 3 to 12 ring carbon atoms (“C₃₋₁₂ cycloalkyl”). In certain embodiments, “carbocyclyl” is a monocyclic, saturated carbocyclyl group having from 3 to 10 ring carbon atoms (“C₃₋₁₀ cycloalkyl”). In certain embodiments, a cycloalkyl group has 3 to 8 ring carbon atoms (“C₃₋₈ cycloalkyl”). In certain embodiments, a cycloalkyl group has 3 to 6 ring carbon atoms (“C₃₋₆ cycloalkyl”). In certain embodiments, a cycloalkyl group has 5 to 6 ring carbon atoms (“C₅₋₆ cycloalkyl”). In certain embodiments, a cycloalkyl group has 5 to 10 ring carbon atoms (“C₅₋₁₀ cycloalkyl”). Examples of C₅₋₆ cycloalkyl include cyclopentyl (C₅) and cyclohexyl (C₅). Examples of C₃₋₆ cycloalkyl include the aforementioned C₅₋₆ cycloalkyl groups as well as cyclopropyl (C₃) and cyclobutyl (C₄). Examples of C₃₋₈ cycloalkyl include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl.

As the foregoing examples illustrate, in certain embodiments, the carbocyclyl group is either monocyclic (“monocyclic carbocyclyl”) or contains a fused, bridged or spiro ring system such as a polycyclic system (“polycyclic carbocyclyl”) and can be saturated or can be partially unsaturated. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continues to designate the number of carbons in the carbocyclyl ring system. When substitution is indicated in such instances, unless otherwise specified, substitution can occur on either the carbocyclyl or the one or more aryl or heteroaryl groups. Unless otherwise specified, each instance of a carbocyclyl group is independently optionally substituted, i.e., unsubstituted (an “unsubstituted carbocyclyl”) or substituted (a “substituted carbocyclyl”) with one or more substituents. In certain embodiments, the carbocyclyl group is unsubstituted C₃₋₁₀ carbocyclyl. In certain embodiments, the carbocyclyl group is a substituted C₃₋₁₀ carbocyclyl.

“Fused carbocyclyl” refers to ring systems wherein the carbocyclyl ring, as defined above, is fused with one or more carbocyclyl ring. In such instances, the number of carbons designates the total number of carbons in the entire fused ring system. When substitution is indicated, unless otherwise specified, substitution can occur on the entire fused ring system. “Fused carbocyclyl” also includes ring systems wherein the carbocyclyl ring, as defined above, is fused with one or more heteroaryl or aryl rings, wherein the point of attachment is on the carbocyclyl ring. In such instances, the number of carbons continues to designate the number of carbons in the carbocyclyl ring. When substitution is indicated, unless otherwise specified, substitution can occur either on the carbocyclyl ring or on the one or more heteroaryl or aryl ring. “Fused carbocyclyl” further includes ring systems wherein the fused carbocyclyl, as defined above, further forms spiro structure with one or more heterocyclyl or carbocyclyl, wherein the point of attachment is on the fused carbocyclyl. In such instances, the number of carbons continues to designate the number of carbons in the fused carbocyclyl. When substitution is indicated in such instances, unless otherwise specified, substitution can occur on either the fused carbocyclyl or the one or more heterocyclyl or carbocyclyl.

“Heterocyclyl” refers to a radical of a 3- to 12-membered non-aromatic ring system having ring carbon atoms and 1 to 4 ring heteroatoms, wherein each heteroatom is independently selected from nitrogen, oxygen, sulfur, boron, phosphorus, and silicon (“3- to 12-membered heterocyclyl”). In heterocyclyl groups that contain one or more nitrogen atoms, the point of attachment can be a carbon or nitrogen atom, as valency permits. Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

In certain embodiments, a heterocyclyl group is a 5- to 12-membered non-aromatic ring system having ring carbon atoms and 1-4 ring heteroatoms, wherein each heteroatom is independently selected from nitrogen, oxygen, sulfur, boron, phosphorus, and silicon (“5- to 12-membered heterocyclyl”). In certain embodiments, a heterocyclyl group is a 5- to 10-membered non-aromatic ring system having ring carbon atoms and 1-4 ring heteroatoms, wherein each heteroatom is independently selected from nitrogen, oxygen, sulfur, boron, phosphorus, and silicon (“5- to 10-membered heterocyclyl”). In certain embodiments, a heterocyclyl group is a 5- to 8-membered non-aromatic ring system having ring carbon atoms and 1-4 ring heteroatoms, wherein each heteroatom is independently selected from nitrogen, oxygen, and sulfur (“5- to 8-membered heterocyclyl”). In certain embodiments, a heterocyclyl group is a 5- to 6-membered non-aromatic ring system having ring carbon atoms and 1-4 ring heteroatoms, wherein each heteroatom is independently selected from nitrogen, oxygen, and sulfur (“5- to 6-membered heterocyclyl”). In certain embodiments, the 5- to 6-membered heterocyclyl has 1-3 ring heteroatoms selected from nitrogen, oxygen, and sulfur. In certain embodiments, the 5- to 6-membered heterocyclyl has 1-2 ring heteroatoms selected from nitrogen, oxygen, and sulfur. In certain embodiments, the 5- to 6-membered heterocyclyl has one ring heteroatom selected from nitrogen, oxygen, and sulfur.

As the foregoing examples illustrate, in certain embodiments, a heterocyclyl group can either be monocyclic (“monocyclic heterocyclyl”) or a fused, bridged or spiro ring system such as a polycyclic system (“polycyclic heterocyclyl”), and can be saturated or can be partially unsaturated. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, and in such instances, the number of ring members designates the total number of ring members in the entire ring system. When substitution is indicated in such instances, unless otherwise specified, substitution can occur on either the heterocyclyl or the one or more carbocyclyl groups. “Heterocyclyl” also includes ring systems wherein the heterocyclyl ring, as defined above, is fused with one or more aryl or heteroaryl groups, wherein the point of attachment is on the heterocyclyl ring, and in such instances, the number of ring members continues to designate the number of ring members in the heterocyclyl ring system. When substitution is indicated in such instances, unless otherwise specified, substitution can occur on either the heterocyclyl or the one or more aryl or heteroaryl groups. Unless otherwise specified, each instance of heterocyclyl is independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

“Fused heterocyclyl” refers to ring systems wherein the heterocyclyl, as defined above, is fused with one or more heterocyclyl or carbocyclyl, wherein the point of attachment is either on the heterocyclyl or on the one or more heterocyclyl or carbocyclyl. In such instances, the number of ring members designates the total number of ring members in the entire ring system. When substitution is indicated in such instances, unless otherwise specified, substitution can occur on the entire ring system. “Fused heterocyclyl” also includes ring systems wherein the heterocyclyl ring, as defined above, is fused with one or more aryl or heteroaryl rings, wherein the point of attachment is on the heterocyclyl ring, and in such instances, the number of ring members continues to designate the number of ring members in the heterocyclyl ring system. When substitution is indicated in such instances, unless otherwise specified, substitution can occur on either the heterocyclyl or the one or more aryl or heteroaryl rings. “Fused heterocyclyl” further includes ring systems wherein the fused heterocyclyl, as defined above, further forms a spiro structure with one or more heterocyclyl or carbocyclyl, wherein the point of attachment is on the fused heterocyclyl. In such instances, the number of ring members continues to designate the number of members in the fused heterocyclyl. When substitution is indicated in such instances, unless otherwise specified, substitution can occur on either the fused heterocyclyl or the one or more heterocyclyl or carbocyclyl to which fused heterocyclyl forms spiro structure.

“Hetero” when used to describe a compound or a group present on a compound means that one or more carbon atoms in the compound or group have been replaced by a nitrogen, oxygen, sulfur, boron, phosphorus, and silicon heteroatom, as valency permits. Hetero may be applied to any of the hydrocarbyl groups described above having from 1 to 5, and particularly from 1 to 3 heteroatoms.

“Alkoxy” as used herein, refers to the group —OR, wherein R is alkyl as defined herein. C₁₋₆ alkoxy refers to the group —OR, wherein each R is C₁₋₆ alkyl, as defined herein. Exemplary C₁₋₆ alkyl is set forth above.

“Alkylamino” as used herein, refers to the group —NHR or —NR₂, wherein each R is independently alkyl, as defined herein. C₁₋₆ alkylamino refers to the group —NHR or —NR₂, wherein each R is independently C₁₋₆ alkyl, as defined herein. Exemplary C₁₋₆ alkyl is set forth above.

“Azido” refers to the radical —N₃.

“Amino” refers to the radical —NH₂.

“Oxo” refers to ═O. When a group other than aryl and heteroaryl or an atom is substituted with an oxo, it is meant to indicate that two geminal radicals on that group or atom form a double bond with an oxygen radical. When a heteroaryl is substituted with an oxo, it is meant to indicate that a resonance/tautomeric structure involving a heteroatom provides a carbon atom that is able to form two geminal radicals, which form a double bond with an oxygen radical.

“Thioketo” refers to the group ═S.

“Carboxy” refers to the radical —C(═O)OH.

“Cyano” refers to the radical —CN.

“Halo” or “halogen” refers to fluoro (F), chloro (Cl), bromo (Br), and iodo (I). In certain embodiments, the halo group is either fluoro or chloro.

“Hydroxy” refers to the radical —OH.

“Nitro” refers to the radical —NO₂.

“Protecting group” as used herein is art-recognized and refers to a chemical moiety introduced into a molecule by chemical modification of a functional group to obtain chemoselectivity in a subsequent chemical reaction, during which the unmodified functional group may not survive or may interfere with the chemical reaction. Common functional groups that need to be protected include but not limited to hydroxyl, amino, thiol, and carboxylic acid. Accordingly, the protecting groups are termed hydroxyl-protecting groups, amino-protecting groups, thiol-protecting groups, and carboxylic acid-protecting groups, respectively.

Common types of hydroxyl-protecting groups include but not limited to ethers (e.g., methoxymethyl (MOM), β-Methoxyethoxymethyl (MEM), tetrahydropyranyl (THP), p-methoxyphenyl (PMP), t-butyl, triphenylmethyl (Trityl), allyl, and benzyl ether (Bn)), silyl ethers (e.g., t-butyldiphenylsilyl (TBDPS), trimethylsilyl (TMS), triisopropylsilyl (TIPS), tri-iso-propylsilyloxymethyl (TOM), and t-butyldimethylsilyl (TBDMS)), and esters (e.g., pivalic acid ester (Piv) and benzoic acid ester (benzoate; Bz)).

Common types of amino-protecting groups include but not limited to carbamates (e.g., t-butyloxycarbonyl (Boc), 9-fluorenylmethyloxycarbonyl (Fmoc), p-methoxybenzyl carbonyl (Moz or MeOZ), 2,2,2-trichloroehtoxycarbonyl (Troc), and benzyl carbamate (Cbz)), esters (e.g., acetyl (Ac); benzoyl (Bz), trifluoroacetyl, and phthalimide), amines (e.g, benzyl (Bn), p-methoxybenzyl (PMB), p-methoxyphenyl (PMP), and triphenylmethyl (trityl)), and sulfonamides (e.g., tosyl (Ts), N-alkyl nitrobenzenesulfonamides (Nosyl), and 2-nitrophenylsulfenyl (Nps)).

Common types of thiol-protecting groups include but not limited to sulfide (e.g., p-methylbenzyl (Meb), t-butyl, acetamidomethyl (Acm), and triphenylmethyl (Trityl)).

Common types of carboxylic acid-protecting groups include but not limited to esters (e.g., methyl ester, triphenylmethyl (Trityl), t-butyl ester, benzyl ester (Bn), S-t-butyl ester, silyl esters, and orthoesters) and oxazoline.

These and other exemplary substituents are described in more detail in the Detailed Description, Examples, and claims. The disclosure is not intended to be limited in any manner by the above exemplary listing of substituents.

### Other Definitions

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of the disclosure that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, chlorobenzenesulfonic acid, 2-naphthalenesulfonic acid, 4-toluenesulfonic acid, camphorsulfonic acid, 4-methylbicyclo [2.2.2]2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium potassium, calcium, magnesium, ammonium, tetraalkylammonium, and the like; and when the compound contains a basic functionality, salts of nontoxic organic or inorganic acids, such as hydrochloride, hydrobromide, tartrate, mesylate, acetate, maleate, oxalate and the like.

The term “pharmaceutically acceptable cation” refers to an acceptable cationic counterion of an acidic functional group. Such cations are exemplified by sodium, potassium, calcium, magnesium, ammonium, tetraalkylammonium cations, and the like (see, e.g., Berge, et al., J. Pharm. Sci. 66 (1): 1-79 (January 77).

“Pharmaceutically acceptable vehicle” refers to a diluent, adjuvant, excipient or carrier with which a compound of the disclosure is administered.

“Pharmaceutically acceptable metabolically cleavable group” refers to a group which is cleaved in vivo to yield the parent molecule of the structural formula indicated herein. Examples of metabolically cleavable groups include —COR, —COOR, —CONR₂ and —CH₂OR radicals, where R is selected independently at each occurrence from alkyl, trialkylsilyl, carbocyclic aryl or carbocyclic aryl substituted with one or more of alkyl, halogen, hydroxy or alkoxy. Specific examples of representative metabolically cleavable groups include acetyl, methoxycarbonyl, benzoyl, methoxymethyl and trimethylsilyl groups.

The term “prodrug,” as used in this disclosure, means a compound which is convertible in vivo by metabolic means (e.g., by hydrolysis) to a disclosed compound.

Since prodrugs may enhance numerous desirable qualities of pharmaceuticals (e.g., solubility, bioavailability, manufacturing, etc.), the compounds of the present disclosure (e.g., a compound of any of the formulae or any individual compounds disclosed herein), or pharmaceutically acceptable salts, solvates, stereoisomers, or tautomers thereof can be delivered in prodrug form. Thus, the present disclosure is intended to cover prodrugs of a compound of the present disclosure (e.g., a compound of any of the formulae or any individual compounds disclosed herein), or a pharmaceutically acceptable salt, solvate, stereoisomer, or tautomer thereof, methods of delivering the same and compositions containing the same. “Prodrugs” are intended to include any covalently bonded carriers that release an active parent drug of the present disclosure in vivo when such prodrug is administered to a mammalian subject. Prodrugs are prepared by modifying functional groups present in the compound in such a way that the modifications are cleaved, either in routine manipulation or in vivo, to the parent compound. Prodrugs include compounds of the disclosure wherein a hydroxyl or amino, group is bonded to any group that, when the prodrug of the present disclosure is administered to a mammalian subject, it cleaves to form a free hydroxyl or free amino group, respectively. Examples of prodrugs include, but are not limited to, acetate, formate, and benzoate derivatives of alcohol and amine functional groups in the compounds of each of the formulae described herein or a pharmaceutically acceptable salt, solvate, stereoisomer, or tautomer thereof.

A “subject” to which administration is contemplated includes, but is not limited to, humans (i.e., a male or female of any age group, e.g., a pediatric subject (e.g, infant, child, adolescent) or an adult subject (e.g., young adult, middle aged adult or senior adult) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal.

An “effective amount” means the amount of a compound that, when administered to a subject for treating or preventing a disease, is sufficient to affect such treatment or prevention. The “effective amount” can vary depending on the compound, the disease and its severity, and the age, weight, etc., of the subject to be treated. A “therapeutically effective amount” refers to the effective amount for therapeutic treatment. A “prophylatically effective amount” refers to the effective amount for prophylactic treatment.

“Preventing”, “prevention” or “prophylactic treatment” refers to a reduction in risk of acquiring or developing a disease or disorder (i.e., causing at least one of the clinical symptoms of the disease not to develop in a subject not yet exposed to a disease-causing agent, or in a subject who is predisposed to the disease in advance of disease onset).

The term “prophylaxis” is related to “prevention,” and refers to a measure or procedure the purpose of which is to prevent, rather than to treat or cure a disease. Non limiting examples of prophylactic measures may include the administration of vaccines; the administration of low molecular weight heparin to hospital patients at risk for thrombosis due, for example, to immobilization, and the administration of an anti-malarial agent such as chloroquine, in advance of a visit to a geographical region where malaria is endemic or the risk of contracting malaria is high.

“Treating” or “treatment” or “therapeutic treatment” of any disease or disorder refers, in one embodiment, to ameliorating the disease or disorder (i.e., arresting the disease or reducing the manifestation, extent or severity of at least one of the clinical symptoms thereof). In another embodiment, “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In a further embodiment, “treating” or “treatment” relates to slowing the progression of the disease.

The term “about” when referring to a number or a numerical range means that the number or numerical range referred to is an approximation within experimental variability or within statistical experimental error, and thus the number or numerical range, in some instances, will vary between 1% and 15% of the stated number or numerical range. In certain embodiments, the number or numerical range vary by 1%, 2%, 3%, 4%, 5%, 6%, 7%, 8%, 9%, 10%, 11%, 12%, 13%, 14%, or 15% of the stated number or numerical range. In certain embodiments, the number or numerical range vary by 1%, 2%, 3%, 4%, or 5% of the stated number or numerical range. In certain embodiments, the number or numerical range vary by 1%, 2%, or 3% of the stated number or numerical range.

The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” may refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) may refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

The claims should not be read as limited to the described order or elements unless stated to that effect. It should be understood that various changes in form and detail may be made by one of ordinary skill in the art without departing from the spirit and scope of the appended claims. All embodiments that come within the spirit and scope of the following claims and equivalents thereto are claimed.

### EXAMPLES

In order that the disclosure described herein may be more fully understood, the following examples are set forth. The examples described in this application are offered to illustrate the compounds, pharmaceutical compositions, and methods provided herein and are not to be construed in any way as limiting their scope.

### I. Synthesis and Characterization

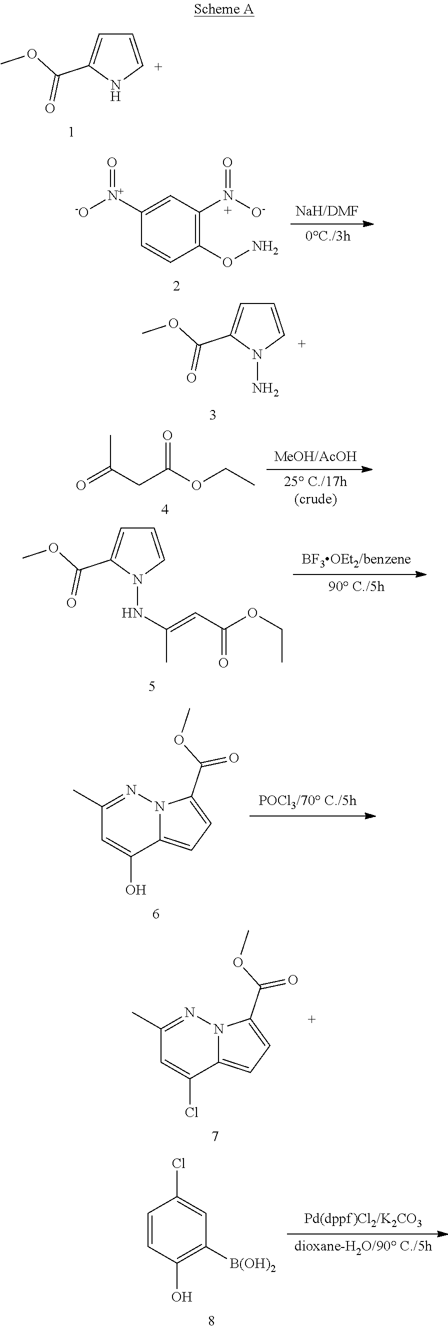

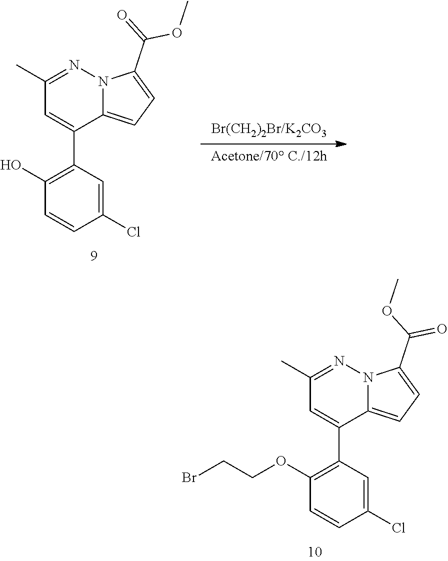

### Synthesis of 3:

To a stirred solution of methyl 1H-pyrrole-2-carboxylate (1) (10 g, 79.92 mmol) in DMF (120 ml) was added sodium hydride (60%, 5.51 g, 239.76 mmol) at 0° C. in portion wise and the reaction mixture was stirred for 1 h at the same temperature. Then O-(2,4-dinitrophenyl) hydroxylamine (2) (23.87 g, 119.88 mmol) in DMF (30 ml) was added drop wise at 0° C. and the reaction mixture was stirred for 3 h at the same temperature. After completion of reaction, the reaction mixture was diluted with saturated aqueous sodium thiosulfate solution (1000 ml), extracted with EtOAc (4×1000 ml). The combined organic layer was washed with brine (2×1000 ml), dried over anhydrous sodium sulphate, filtered and concentrated under reduced pressure to get crude. The crude residue was purified by combiflash column purification (SiO₂, 120 g, 5% EtOAc in Hexanes) to afforded methyl 1-azanylpyrrole-2-carboxylate (3) (11 g, 78.49 mmol, 98.21% yield) as a light-yellow sticky liquid.

1H NMR (400 MHz, DMSO-d6): 7.02-7.01 (m, 1H), 6.71-6.70 (m, 1H), 6.25 (s, 2H), 5.98-5.96 (m, 1H), 3.74 (s, 3H) ppm.

LCMS: LC/MS was submitted, but compound wasn't ionized. Hence not included in the analytical traces.

### Synthesis of 5:

To a stirred solution of methyl 1-azanylpyrrole-2-carboxylate (3) (5 g, 35.68 mmol) in methanol (200 ml) in were added ethyl 3-oxidanylidenebutanoate (4) (5.57 g, 42.81 mmol, 5.44 mL) followed by acetic acid (50 ml) at 25° C. and the reaction mixture was stirred at 25° C. for 16 h. After completion of reaction, the volatiles were removed under reduced pressure and crude was diluted with aqueous saturated NaHCO₃ solution. The aqueous was extracted with EtOAc, the combined organic layer was washed with brine, dried over anhydrous sodium sulphate, filtered and concentrated under reduced pressure to obtain methyl 1-[[(E)-3-ethoxy-1-methyl-3-oxidanylidene-prop-1-enyl]amino]pyrrole-2-carboxylate (5) (8 g, crude) as a yellow oil, which was forwarded to next step without further purification.

¹HNMR: not recorded

LCMS: Column-Xbridge C18 (50×4.6 mm, 5 u, 130A), (mobile phase: from 90% [10 mM NH4OAc in water] and 10% [CH3CN] to 70% [10 mM NH4OAc in water] and 30% [CH3CN] in 1.5 min, further to 10% [10 mM NH4OAc in water] and 90% [CH3CN] in 3.0 min, held this mobile phase composition up to 4.0 min and finally back to initial condition in 5.0 min). Flow=1.2 ml/min. Rt=3.17 and 3.40 (5 min run), MS calculated: 252.2; MS found: 253.2 (M+H).

### Synthesis of 6:

To a stirred solution of methyl 1-[[(E)-3-ethoxy-1-methyl-3-oxidanylidene-prop-1-enyl]amino]pyrrole-2-carboxylate (5) (8 g, 31.71 mmol) in benzene (80 ml) was added boron trifluoride diethyl etherate (13.50 g, 95.14 mmol, 11.7 ml) slowly at 25° C. and the reaction mixture was stirred for 5 h at 90° C. The reaction mixture cooled to 25° C. and stirred it for another 17 h at the same temperature. After completion of reaction, the reaction mixture was quenched with water and extracted with ethyl acetate. The combined organic part was washed with brine and dried over anhydrous sodium sulphate, filtered and concentrated under reduced pressure. The crude which was purified in combiflash column chromatography (SiO₂; EtOAc:Hexanes 70%) to afford methyl 2-methyl-4-oxidanyl-pyrrolo[1,2-b]pyridazine-7-carboxylate (6) (2.5 g, 12.12 mmol, 38.23% yield) as an off white solid.

1H NMR (400 MHz, DMSO-d6): 11.72 (s, 1H), 7.28 (d, J=4.76 Hz, 1H), 6.61 (d, J=4.8 Hz, 1H), 6.19 (s, 1H), 3.78 (s, 3H), 2.39 (s, 3H) ppm.

LCMS: Column-Xbridge C18 (50×4.6 mm, 5 u, 130A), (mobile phase: from 90% [10 mM NH4OAc in water] and 10% [CH3CN] to 70% [10 mM NH4OAc in water] and 30% [CH₃CN] in 1.5 min, further to 10% [10 mM NH4OAc in water] and 90% [CH₃CN] in 3.0 min, held this mobile phase composition up to 4.0 min and finally back to initial condition in 5.0 min). Flow=1.2 ml/min. Rt=1.48 MS calculated: 206; MS found: 207 (M+H).

### Synthesis of 7:

To a mixture of phosphorus oxychloride (37.18 g, 242.49 mmol, 22.7 mL) and methyl 2-methyl-4-oxidanyl-pyrrolo[1,2-b]pyridazine-7-carboxylate (6) (2.5 g, 12.12 mmol) was heated at 70° C. for 5 h. After completion of reaction, the volatiles were removed under reduced pressure. The crude was purified in combiflash column chromatography (SiO₂; 40 g, 10% EtOAc:Hexane) to afford methyl 4-chloranyl-2-methyl-pyrrolo[1,2-b]pyridazine-7-carboxylate (7) (1.9 g, 8.46 mmol, 69.76% yield) as an off white solid.

1H NMR (400 MHz, DMSO-d6): 7.48 (d, J=4.6 Hz, 1H), 7.27 (s, 1H), 6.74 (d, J=4.6 Hz, 1H), 3.82 (s, 3H), 2.5 (s, 3H) ppm.

LCMS: Column-Xbridge C18 (50×4.6 mm, 5 u, 130A), (mobile phase: from 90% [10 mM NH4OAc in water] and 10% [CH3CN] to 70% [10 mM NH4OAc in water] and 30% [CH3CN] in 1.5 min, further to 10% [10 mM NH4OAc in water] and 90% [CH3CN] in 3.0 min, held this mobile phase composition up to 4.0 min and finally back to initial condition in 5.0 min). Flow=1.2 ml/min. Purity is 99.81%, Rt=3.38 min, MS calculated: 224; MS found: 224.8 (M+H).

### Synthesis of 9:

To a stirred solution mixture of methyl 4-chloranyl-2-methyl-pyrrolo[1,2-b] pyridazine-7-carboxylate (1.9 g, 8.46 mmol) (7) and (5-chloranyl-2-oxidanyl-phenyl) boronic acid (8) (1.75 g, 10.15 mmol) in water (2 ml) & dioxane (20 ml) was added potassium carbonate (4.09 g, 29.60 mmol) at 25° C., and the reaction mixture was degassed with argon for 30 min. To this was added Pd(dppf)Cl₂ (1.86 g, 2.54 mmol) and the reaction mixture was again degassed with argon for 10 min. The reaction mixture was stirred at 90° C. for 5 h. After completion of reaction, the reaction mixture was filtered through celite bed and washed with EtOAc (2×30 ml). The organic layer was dried with anhydrous sodium sulphate, filtered and concentrated under reduced pressure. The crude was purified by combiflash column chromatography (SiO₂; 12 g, 50% EtOAc/hexane as eluent) to afford methyl 4-(5-chloranyl-2-oxidanyl-phenyl)-2-methyl-pyrrolo[1,2-b]pyridazine-7-carboxylate (9) (2.1 g, 6.63 mmol, 78.39% yield) as a light yellow solid.

1H NMR (400 MHz, DMSO-d6): 10.14 (s, 1H), 7.41-7.37 (m, 3H), 6.95 (s, 1H), 6.32 (d, 1H, J=4.4), 3.82 (s, 3H), 2.53 (s, 3H) ppm.

LCMS: Column-Xbridge C18 (50×4.6 mm, 5 u, 130A), (mobile phase: from 90% [10 mM NH4OAc in water] and 10% [CH3CN] to 70% [10 mM NH4OAc in water] and 30% [CH3CN] in 1.5 min, further to 10% [10 mM NH4OAc in water] and 90% [CH3CN] in 3.0 min, held this mobile phase composition up to 4.0 min and finally back to initial condition in 5.0 min). Flow=1.2 ml/min. Purity is 97.11%, Rt=3.36 min, MS calculated: 316; MS found: 315.2 (M−H).

### Synthesis of 10:

To a stirred solution of methyl 4-(5-chloranyl-2-oxidanyl-phenyl)-2-methyl-pyrrolo[1,2-b]pyridazine-7-carboxylate (9) (5 g, 15.79 mmol) in acetone (50 ml) was added 1,2-dibromoethane (29.66 g, 157.86 mmol, 13.60 ml) followed by potassium carbonate (7.64 g, 55.25 mmol) at 25° C. under nitrogen then stirred at 70° C. for 12 h. After completion of reaction, the insoluble material was filtered through sintered funnel and the filtrate was concentrated under reduced pressure. The crude was purified by combiflash column chromatography (SiO₂; 40 g, 70% EtOAc/Hexane) to afford methyl 4-[2-(2-bromanylethoxy)-5-chloranyl-phenyl]-2-methyl-pyrrolo[1,2-b]pyridazine-7-carboxylate (10) (3.5 g, 8.18 mmol, 51.81% yield, 99% purity) as an off white solid.

1H NMR (400 MHz, DMSO-d6): 7.56-7.53 (m, 2H), 7.41 (d, 1H, J=4.68), 7.27 (d, 1H, J=8.52), 7.02 (s, 1H), 6.36 (d, 1H, J=4.4), 4.37-4.36 (m, 2H), 3.82 (s, 3H), 3.64-3.63 (m, 2H), 2.54 (s, 3H) ppm.

LCMS: Column-Xbridge C18 (3×50 mm, 3.5 u) (mobile phase: 95% [5 mM NH4OAc in water] and 5% [5 mM NH4OAc in ACN: Water (90:10)] held for 0.75 min, then to 70% [5 mM NH4OAc in water] and 30% [5 mM NH4OAc in ACN: Water (90:10)] in 1.00 min, and finally 2% [5 mM NH4OAc in water] and 98% [5 mM NH4OAc in ACN: Water (90:10)] in 2.00 min, held this mobile phase composition up to 2.50 min and finally back to initial condition in 2.75 min and held this composition up to 3.0 min). Flow rate—1.20 ml/min. Purity is 98.77%, Rt=2.17 min, MS calculated: 422; MS found: 423.1 (M+H).

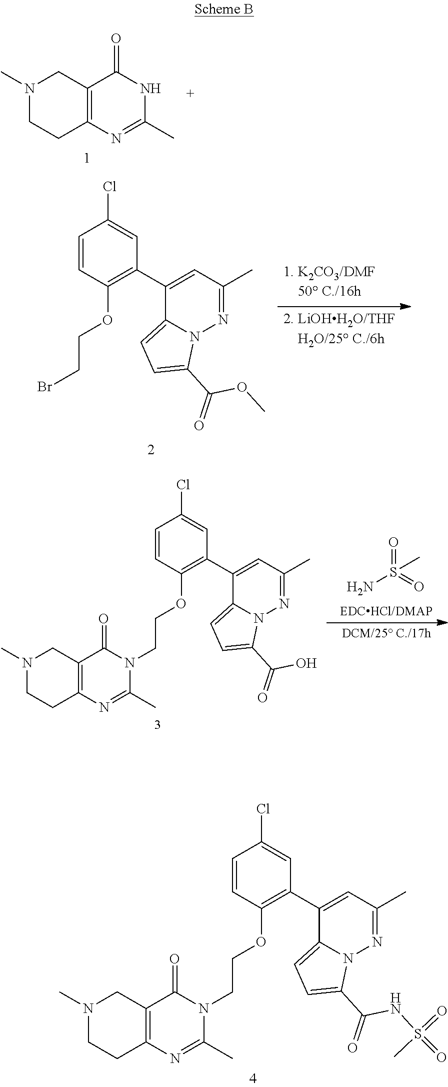

### Syntheses of 3 (3) (Compound 26)

To a stirred solution of 2,6-di(methyl)-3,5,7,8-tetrahydropyrido[4,3-d]pyrimidin-4-one (1) (300 mg, 1.67 mmol) in DMF (10 ml) was added potassium carbonate-granular (694 mg, 5.02 mmol) at 10° C. under argon and the reaction mixture was stirred for 20 min at 10° C. To this was added methyl 4-[2-(2-bromanylethoxy)-5-chloranyl-phenyl]-2-methyl-pyrrolo[1,2-b]pyridazine-7-carboxylate (2) (709 mg, 1.67 mmol) at that temperature and the reaction was stirred at 50° C. for 17 h. After completion of reaction, the reaction mixture was quenched with water. The aqueous phase was extracted with EtOAc. The combined organic layer was washed with brine, dried over anhydrous Na₂SO₄, filtered and filtrate was evaporated under reduced pressure. The crude was thus obtained was subjected to flash column chromatography. To a stirred solution of the impure solid (450 mg, 862.08 μmol) in a mixture of THF (8 ml), methanol (4 ml) & H₂O (2 ml) was added lithium hydroxide monohydrate (181 mg, 4.31 mmol) at 25° C., and the mixture was stirred at 25° C. for 2 h. After completion of reaction (as judged by LC/MS and TLC), solvents were removed under reduced pressure and the residue was diluted with water. The aqueous part was acidified with aqueous HCl (1M) to pH˜2-3 and extracted with EtOAc (2×30 ml). The combined organic layer was dried over anhydrous Na₂SO₄, filtered and filtrate was concentrated under reduced pressure. The crude residue was purified by preparative HPLC to afforded 4-[5-chloranyl-2-[2-[2,6-di(methyl)-4-oxidanylidene-7,8-dihydro-5H-pyrido[4,3-d]pyrimidin-3-yl]ethoxy]phenyl]-2-methyl-pyrrolo[1,2-b]pyridazine-7-carboxylic acid (3) (Compound 26) (130 mg, 231.48 μmol, 26.85% yield, 90.45% purity) as a white solid.

1H NMR (400 MHz, DMSO-d6): 7.55-7.52 (m, 1H), 7.41 (d, J=2.5 Hz, 1H), 7.28-7.23 (m, 2H), 6.83 (s, 1H), 6.07 (d, J=4.8 Hz, 1H), 4.29-4.27 (m, 2H), 4.11-4.09 (m, 2H), 3.08 (s, 2H), 2.55 (s, 3H), 2.32 (s, 3H), 1.77 (s, 3H).

### Synthesis of 4 (4) (Compound 27)

To a stirred solution of 4-[5-chloranyl-2-[2-[2,6-di(methyl)-4-oxidanylidene-7,8-dihydro-5H-pyrido[4,3-d]pyrimidin-3-yl]ethoxy]phenyl]-2-methyl-pyrrolo[1,2-b]pyridazine-7-carboxylic acid (3) (Compound 26) (75 mg, 147.65 μmol) in dichloromethane (7 ml) were added DMAP (90 mg, 738.24 μmol) followed by EDC·HCl (57 mg, 295.29 μmol) at 25° C. and the reaction mixtures was stirred for 30 min at same temperature. To this was added methanesulfonamide (70 mg, 738.24 μmol) at 25° C. and the reaction mixture was stirred for 16 h at 25° C. After completion of reaction, the reaction mixture was diluted with DCM and washed with water, brine, dried over anhydrous Na₂SO₄, filtered and the filtrate was concentrated under reduced pressure. The crude was purified by RP preparative HPLC to get 4-[5-chloranyl-2-[2-[2,6-di(methyl)-4-oxidanylidene-7,8-dihydro-5H-pyrido[4,3-d]pyrimidin-3-yl]ethoxy]phenyl]-2-methyl-N-methylsulfonyl-pyrrolo[1,2-b]pyridazine-7-carboxamide (4) (Compound 27) (44 mg, 74.63 μmol, 50.55% yield, 99.24% purity) as a white solid.

1H NMR (400 MHz, DMSO, at 100° C.): δ 7.55-7.52 (m, 1H), 7.43-7.39 (m, 1H), 7.28 (d, J=9.2 Hz, 1H), 6.91 (s, 1H), 6.22 (d, J=4.4 Hz, 1H), 4.32-4.30 (m, 2H), 4.15-4.12 (m, 2H), 3.43 (s, 3H), 3.12 (s, 3H), 2.61-2.58 (m, 5H), 2.37 (s, 3H), 1.94 (s, 3H) ppm.

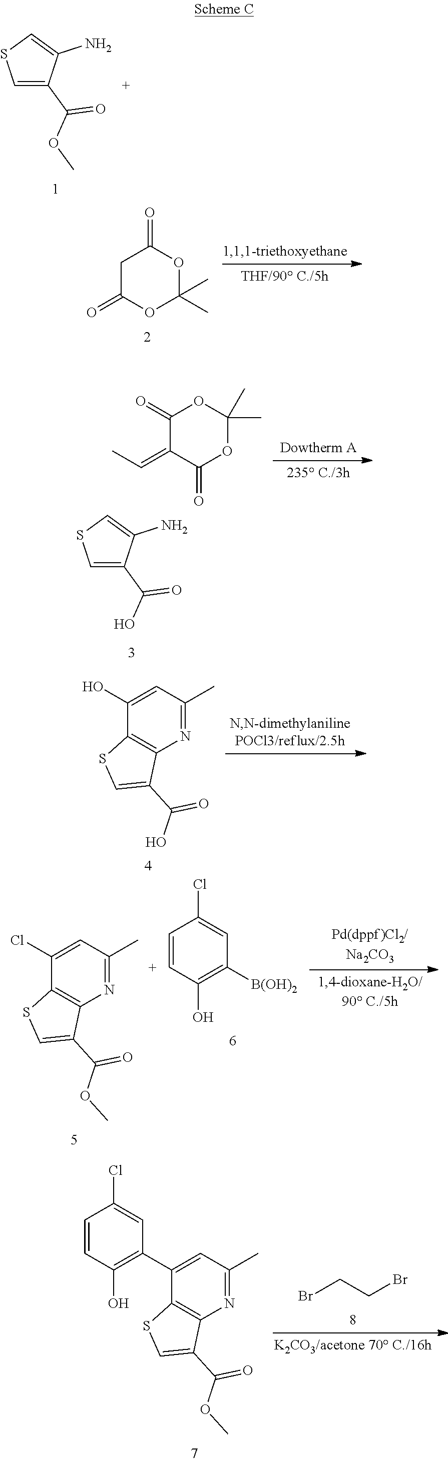

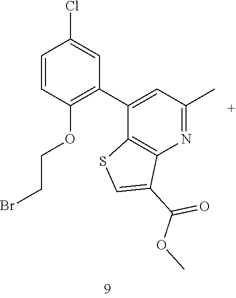 [SMILES: COC(=O)c1csc2c(-c3cc(Cl)ccc3OCCBr)cc(C)nc12]

### Synthesis of 3:

To a stirred solution of 2,2-di(methyl)-1,3-dioxane-4,6-dione (2) (7.06 g, 48.99 mmol) in 1,1,1-triethoxyethane (7.22 g, 44.53 mmol, 50 mL) was heated at 90° C. for 3 h. After consumption of starting (judged by TLC) the solvent was evaporated and the reaction mixture was dissolved in THF (50 mL). Then, methyl 4-azanylthiophene-3-carboxylate (1) (7 g, 44.53 mmol) was added to the reaction mixture and heating was continued at 90° C. for 2 h. After consumption of starting material, the reaction mixture was quenched with water and extracted with ethyl acetate (150 mL). The organic layer was washed with brine solution, dried over anhydrous sodium sulphate and concentrated under reduced pressure. Combiflash chromatography was done (SiO2, 120 g, 40% EtOAc/hexanes) to give methyl 4-[1-[2,2-di(methyl)-4,6-bis(oxidanylidene)-1,3-dioxan-5-ylidene]ethylamino]thiophene-3-carboxylate (3) (2 g, 6.15 mmol, 14%) as a yellow liquid.

1H NMR (400 MHz, DMSO-d6) δ1.20 (m, 2H), 1.65 (s, 3H), 2.53 (m, 1H), 3.30 (s, 3H), 3.96 (m, 2H), 4.1 (m, 1H), 7.78 (d, J=12.3 Hz, 1H), 8.48 (d, J=3.28 Hz, 1H), 12.68 (s, 1H).

LCMS: LC/MS was submitted, but compound wasn't ionized. Hence not included in the analytical traces.

### Synthesis of 4:

To a stirred solution of methyl 4-[1-[2,2-di(methyl)-4,6-bis(oxidanylidene)-1,3-dioxan-5-ylidene]ethylamino]thiophene-3-carboxylate (3) (20 g, 61.47 mmol) in Dowtherm (19.94 g, 61.47 mmol, 40 mL) was heated at 230° C. for 2 h. After completion of starting material (judged by TLC), the volatiles were removed under reduced pressure. The crude thus obtained was purified combiflash chromatography (SiO2, 120 g, 100% Ethyl acetate) to give methyl 5-methyl-7-oxidanyl-thieno[3,2-b]pyridine-3-carboxylate (4) (6 g, 26.88 mmol, 43.72% yield) as a brown solid.

1H NMR (400 MHz, DMSO-d6) δ 2.43 (s, 3H), 2.54 (s, 1H), 2.56 (s, 1H), 3.85 (s, 1H), 3.91 (s, 3H), 5.99 (s, 1H), 8.78 (s, 1H), 10.95 (s, 1H).

LCMS: LC/MS was submitted, but compound wasn't ionized. Hence not included in the analytical traces.

### Synthesis of 5:

To a stirred solution of methyl 5-methyl-7-oxidanyl-thieno[3,2-b]pyridine-3-carboxylate (4) (3.5 g, 15.68 mmol) in toluene (50 mL) was added N,N-Dimethylaniline, 99% (15.20 g, 125.42 mmol, 15.90 mL) and cooled to 0° C. POCl₃ (3.60 g, 23.52 mmol) was added to the reaction mixture and heated at 120° C. for 2.5 h. After completion of starting material (judged by TLC), the solvent was evaporated under reduced pressure and crude thus obtained was purified by combiflash chromatography (SiO2, 120 g, 30% ethyl acetate/hexane) to get methyl 7-chloranyl-5-methyl-thieno[3,2-b]pyridine-3-carboxylate (5) (3.0 g, 12.41 mmol, 79.17% yield) as an white solid.

1H NMR (400 MHz, DMSO-d6) δ 8.93 (s, 1H), 7.57 (s, 1H), 3.86 (s, 3H), 2.63 (s, 3H).

ESI-MS: m/z calc.241.0, found 243.0 (M+2)+; retention time 3.17 min (5 min run)

### Synthesis of 7:

To a stirred solution of methyl 7-chloranyl-5-methyl-thieno[3,2-b]pyridine-3-carboxylate (5) (3.0 g, 12.41 mmol) in dioxane (30 mL) & water (5 mL) were added (5-chloranyl-2-oxidanyl-phenyl) boronic acid (6) (2.14 g, 12.41 mmol) followed by Na₂CO₃ (3.95 g, 37.24 mmol) under Argon atmosphere and degassed for 10 min at 25° C. To this solution Pd(dppf)₂Cl₂ (907.35 mg, 1.24 mmol) was added and again degassed for 5 mins. Then, the reaction mixture was heated at 90° C. for 5 h. After completion of reaction (confirmed by LCMS), the reaction mixture was filtered through a celite bed and concentrated under reduced pressure. The crude thus obtained was purified by flash column chromatography (SiO2, 40 g, 30 to 35%: ethyl acetae/hexane) to give methyl 7-(5-chloranyl-2-oxidanyl-phenyl)-5-methyl-thieno[3,2-b]pyridine-3-carboxylate (7) (1.5 g, 4.49 mmol, 36.20% yield) as off white solid.

1H NMR (400 MHz, DMSO-d6) δ 10.26 (s, 1H), 8.84 (s, 1H), 7.38 (t, J=8.08 Hz 3H), 7.05 (d, J=8.68 Hz 1H), 3.87 (s, 3H), 2.66 (s, 3H).

ESI-MS: m/z calc. 333, found 334 (M+H)⁺; retention time 3.32 min (5 min run).

### Synthesis of 9:

To a stirred solution of methyl 7-(5-chloranyl-2-oxidanyl-phenyl)-5-methyl-thieno[3,2-b]pyridine-3-pcarboxylate (7) (1.2 g, 3.60 mmol) in acetone (25 mL) was added anhydrous potassium carbonate (1.49 g, 10.79 mmol, 650.91 μL) followed by 1,2-bis(bromanyl)ethane (8) (5.40 g, 28.76 mmol, 2.48 mL) and the reaction mixture was refluxed at 70° C. for 16 h. After completion of reaction (as jugged by LC/MS only), the reaction mixture was cooled to 25° C. and filtered. The filtrate was evaporated under reduced pressure. The crude was thus obtained was purified by silica gel column chromatography (SiO₂; 40 g, 50% EtOAc/Hexanes) to give methyl 7-[2-(2-bromanylethoxy)-5-chloranyl-phenyl]-5-methyl-thieno[3,2-b]pyridine-3-carboxylate (9) (1.3 g, 2.95 mmol, 82.05% yield) as an off white solid.

1H-NMR (400 MHz, DMSO-d6) δ 8.84 (s, 1H), 7.57 (m, 2H), 7.42 (s, 1H), 7.30 (m, 1H), 4.36 (m, 2H), 3.93 (s, 3H), 3.62 (m, 2H), 2.66 (s, 3H).

ESI-MS: m/z calc 440.74; found 441.8 (M+1)-; retention time 3.65 min (5 min run).

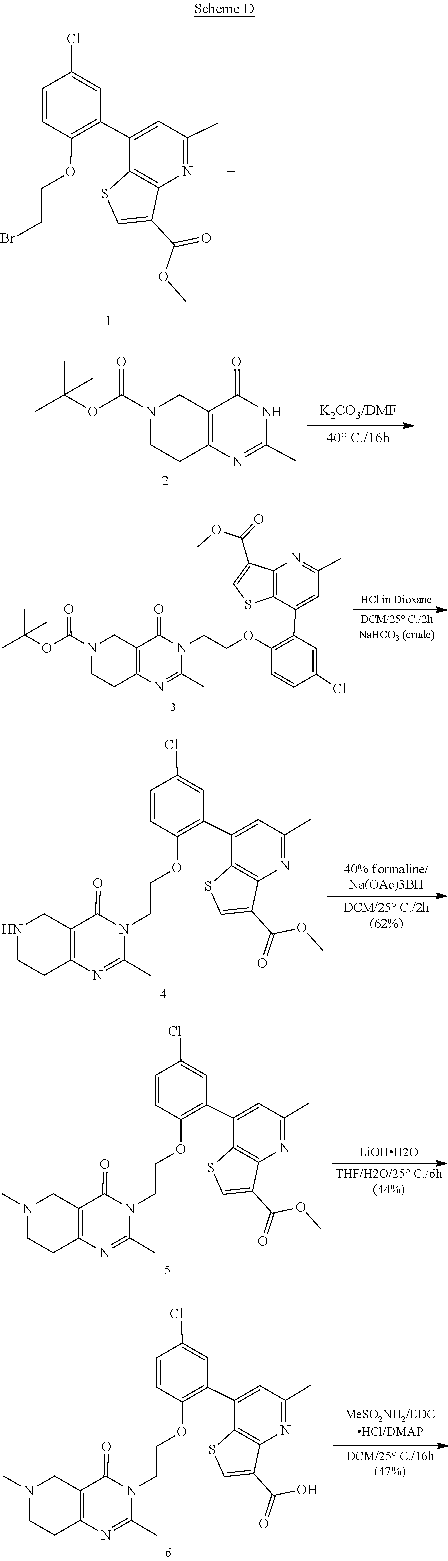

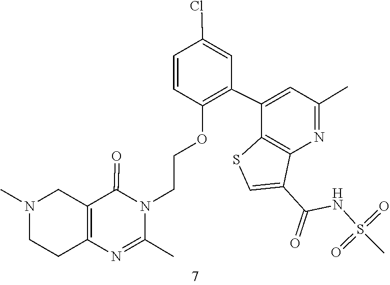 [SMILES: Cc1cc(-c2cc(Cl)ccc2OCCn2c(C)nc3c(c2=O)CN(C)CC3)c2scc(C(=O)NS(C)(=O)=O)c2n1]

### Synthesis of 3:

To a stirred solution of the compound 1,1-di(methyl)ethyl 2-methyl-4-oxidanylidene-3,5,7,8-tetrahydropyrido[4,3-d]pyrimidine-6-carboxylate (1.44 g, 5.45 mmol) (2) in DMF (15 mL) was added dry K₂CO₃ (1.88 g, 13.61 mmol) and the reaction mixture was stirred for 20 min at 25° C. To this was added methyl 7-[2-(2-bromanylethoxy)-5-chloranyl-phenyl]-5-methyl-thieno[3,2-b]pyridine-3-carboxylate (1) (2000 mg, 4.54 mmol) at 25° C. and the reaction mixture was stirred at 25° C. for 16 h. After completion of reaction (as judged by TLC & LC/MS), the reaction mixture was passes through a pad of celite and washed with ethyl acetate. The filtrate was evaporated under reduced pressure and the crude was purified by silica gel combi flash column chromatography (SiO2; 40 g, 5% MeOH/DCM) to afford methyl 7-[5-chloranyl-2-[2-[6-[1,1-di(methyl) ethoxycarbonyl]-2-methyl-4-oxidanylidene-7,8-dihydro-5H-pyrido[4,3-d]pyrimidin-3-yl]ethoxy]phenyl]-5-methyl-thieno[3,2-b]pyridine-3-carboxylate (3) (1 g, 1.50 mmol, 33.14% yield) as an off white solid.

¹H-NMR (400 MHz, DMSO-d₆): 8.66 (s, 1H), 7.54 (d, J=5.9 Hz, 1H), 7.40 (m, 1H), 7.31-7.26 (m, 2H), 4.31 (s, 2H), 4.08 (s, 4H), 3.88 (s, 3H), 3.55-3.50 (m, 2H), 2.66 (s, 3H), 1.60 (s, 3H), 1.42 (s, 9H) ppm.

m/z calc. 624.18, found (M+1)=625.0 at RT 3.86.

### Synthesis of 4:

To a stirred solution of the compound methyl 7-[5-chloranyl-2-[2-[6-[1,1-di(methyl)ethoxycarbonyl]-2-methyl-4-oxidanylidene-7,8-dihydro-5H-pyrido[4,3-d]pyrimidin-3-yl]ethoxy]phenyl]-5-methyl-thieno[3,2-b]pyridine-3-carboxylate (3) (1000 mg, 1.60 mmol) in DCM (6 mL) was added 4 (N) HCl in Dioxane (7 mL) at 0° C. and the reaction mixture was stirred at 25° C. for 4 h. After completion of reaction (as judged by TLC & LC/MS), the solvents were evaporated under reduced pressure to get HCl salt of the desired product which was basified with the saturated solution of NaHCO₃. The aqueous layer was extracted with 10% MeOH/DCM twice. The combined organic layer was dried over Na₂SO₄, filtered and filtrate was evaporated under reduced pressure to get methyl 7-[5-chloranyl-2-[2-(2-methyl-4-oxidanylidene-5,6,7,8-tetrahydropyrido[4,3-d]pyrimidin-3-yl)ethoxy]phenyl]-5-methyl-thieno[3,2-b]pyridine-3-carboxylate (4) (700 mg, 1.33 mmol, 83.35% yield) as a white solid, which was used to next step without further purification.

m/z calc. 524.1, found (M+1)=525.2 at RT 2.89.

### Synthesis of 5:

To a stirred solution of the compound methyl 7-[5-chloranyl-2-[2-(2-methyl-4-oxidanylidene-5,6,7,8-tetrahydropyrido[4,3-d]pyrimidin-3-yl) ethoxy]phenyl]-5-methyl-thieno[3,2-b]pyridine-3-carboxylate (1) (100 mg, 190.47 μmol) in DCM was added 37% formalin (22.86 mg, 761.88 μmol, 61.77 μL) at 0° C. Then the reaction mixture was stirred at 25° C. for 1 h and Na(OAc)3BH (201.84 mg, 952.35 μmol) was added to the mixture at 0° C. The reaction mixture was then stirred for additional 1 h at 25° C. After completion of the reaction (as judged by LC/MS only), the solvents were evaporated under reduced pressure to get the crude material. The crude was thus obtained was purified by (Chromatorex (R) NH DM1020 (mesh 100-200)-Amine silica gel; 12 g, 1% MeOH/DCM) to obtain the compound methyl 7-[5-chloranyl-2-[2-[2,6-di(methyl)-4-oxidanylidene-7,8-dihydro-5H-pyrido[4,3-d]pyrimidin-3-yl]ethoxy]ph-enyl]-5-methyl-thieno[3,2-b]pyridine-3-carboxylate (5) (80 mg, 148.41 μmol, 77.92% yield) as light yellow solid.

m/z calc.538.1, found (M+1)=539.0 at RT 3.37.

### Synthesis of 6 (Compound 29)

To a stirred solution of the compound methyl 7-[5-chloranyl-2-[2-[2,6-di(methyl)-4-oxidanylidene-7,8-dihydro-5H-pyrido[4,3-d]pyrimidin-3-yl]ethoxy]phenyl]-5-methyl-thieno[3,2-b]pyridine-3-carboxylate (5) (80 mg, 148.41 μmol) in a mixture of THF (2.00 mL) and Water (0.5 mL) was added LiOH·H₂O (24.91 mg, 593.64 μmol, 16.50 μL). The reaction mixture was then stirred at 25° C. for 6 h. After completion of reaction (as judged by TLC & LC/MS), the volatiles were removed under reduced pressure. The crude was thus obtained was purified by reverse phase prep-HPLC to afford the pure compound 7-[5-chloranyl-2-[2-[2,6-di(methyl)-4-oxidanylidene-7,8-dihydro-5H-pyrido[4,3-d]pyrimidin-3-yl]ethoxy]phen-yl]-5-methyl-thieno[3,2-b]pyridine-3-carboxylic acid (6) (Compound 29) (34 mg, 64.68 μmol, 43.58% yield, 99.87% purity) as a white solid.

¹H NMR-VT (400 MHz, DMSO-d6): δ 8.77 (s, 1H), 7.55 (dd, J=8.80, 2.80 Hz, 1H), 7.42 (d, J=2.40 Hz, 1H), 7.37 (s, 1H), 7.31 (d, J=8.80 Hz, 1H), 4.33 (t, J=5.2 Hz, 2H), 4.13 (t, J=5.2 Hz, 2H), 3.09 (s, 2H), 2.75 (s, 3H), 2.67 (s, 1H), 2.58 (t, J=5.2 Hz, 2H), 2.35 (s, 3H), 1.80 (s, 3H) ppm.

### Synthesis of 7 (Compound 28)

To a stirred solution of the compounds 7-[5-chloranyl-2-[2-[2,6-di(methyl)-4-oxidanylidene-7,8-dihydro-5H-pyrido[4,3-d]pyrimidin-3-yl]ethoxy]phenyl]-5-methyl-thieno[3,2-b]pyridine-3-carboxylic acid (6) (Compound 29) (100 mg, 190.47 μmol) and MeSO2NH2 (54.35 mg, 571.41 μmol) in DCM (7 mL) were added EDC·HCl (59.14 mg, 380.94 μmol) and DMAP (81.44 mg, 666.64 μmol) at 0° C. Then the reaction mixture was stirred at 25 0° C. for 20 h. After completion of reaction (as judged by TLC & LC/MS), the volatiles were removed under reduced pressure. The crude was thus obtained was purified by reverse phase prep-HPLC to afford the pure compound 7-[5-chloranyl-2-[2-[2,6-di(methyl)-4-oxidanylidene-7,8-dihydro-5H-pyrido[4,3-d]pyrimidin-3-yl]ethoxy]phenyl]-5-methyl-N-methylsulfonyl-thieno[3,2-b]pyridine-3-carboxamide; 2,2,2-tris(fluoranyl)acetic acid (7) (Compound 28) (65 mg, 89.14 μmol, 46.80% yield, 98.21% purity) as white solid.

¹H NMR (400 MHz, DMSO-d6): δ 12.87 (brs, 1H), 8.99 (s, 1H), 7.57 (dd, J=9.2, 2.80 Hz, 1H), 7.45 (s, 1H), 7.43 (d, J=2.4 Hz, 1H), 7.31 (d, J=9.2 Hz, 1H), 4.36 (t, J=4.8 Hz, 2H), 4.15 (t, J=4.8 Hz, 2H), 4.01 (s, 2H), 3.49 (s, 5H), 2.96 (s, 3H), 2.77 (s, 5H), 1.72 (s, 3H) ppm.

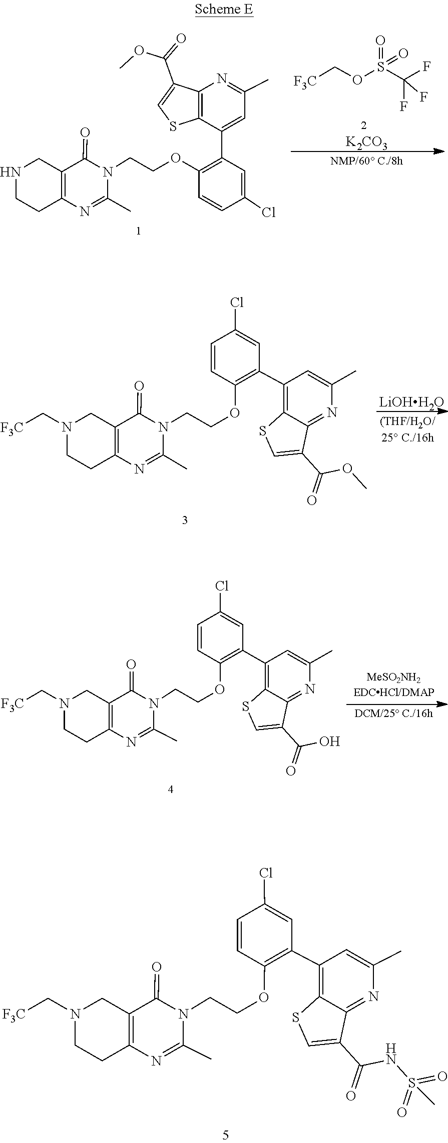

### Synthesis of 3:

To a stirred solution of methyl 7-[5-chloranyl-2-[2-(2-methyl-4-oxidan-ylidene-5,6,7,8-tetrahydropyrido[4,3-d]pyrimidin-3-yl) ethoxy]phenyl]-5-methyl-thieno[3,2-b]pyridine-3-carboxylate (1) (100 mg, 190.47 μmol) in NMP (2 mL) were added K₂CO₃ (105.14 mg, 761.88 μmol) followed by 2,2,2-tris(fluoranyl)ethyl tris(fluoranyl)methanesulfonate (2) (53.05 mg, 228.56 μmol, 32.93 μL) at 25° C. and the reaction mixture was stirred for 16 h at 40° C. After the completion of reaction (as judged by LCMS and TLC analysis), the reaction mixture was diluted with ethyl acetate and washed with ice cooled water, brine, dried over Na₂SO₄, filtered and filtrate was evaporated under reduced pressure to get methyl 7-[5-chloranyl-2-[2-[2-methyl-4-oxidanylidene-6-[2,2,2-tris(fluoranyl)ethyl]-7,8-dihydro-5H-pyrido[4,3-d]pyrimidin-3-yl]ethoxy]phenyl]-5-methyl-thieno[3,2-b]pyridine-3-carboxylate (3) (100 mg, 214.15 μmol, 95% yield) as a light yellow gum, which was used for the next step without any further purification.

m/z calc. 606.1, found (M+1)=607 at RT 3.85.

### Synthesis of 4 (Compound 24)

To a stirred solution of the compound methyl 7-[5-chloranyl-2-[2-[2-methyl-4-oxidanylid-ene-6-[2,2,2-tris(fluoranyl)ethyl]-7,8-dihydro-5H-pyrido[4,3-d]pyrimidin-3-yl]ethoxy]phenyl]-5-methyl-thieno[3,2-b]pyridine-3-carboxylate (3) (100 mg, 165 μmol) in a mixture of THF (2 mL) and water (0.5 mL) was added LiOH·H2O (35.94 mg, 856.61 μmol, 23.80 μL) at 25° C. and the reaction mixture was stirred at 25° C. for 16 h. After completion of reaction (as judged by TLC & LC/MS), the reaction mixture was evaporated under reduced pressure. The crude thus obtained was purified by reverse phase prep-HPLC to give 7-[5-chloranyl-2-[2-[2-methyl-4-oxidany-lidene-6-[2,2,2-tris(fluoranyl)ethyl]-7,8-dihydro-5H-pyrido[4,3-d]pyrimidin-3-yl]ethoxy]phenyl]-5-methyl-thieno[3,2-b]pyridine-3-carboxylic acid; 2,2,2-tris(fluoranyl) acetic acid (4) (Compound 24) (25 mg, 33.15 μmol, 15.48% yield, 93.75% purity) as an off white sticky solid.

¹H NMR (400 MHz, DMSO-d6): δ 8.79 (s, 1H), 7.55 (dd, J=8.80, 2.80 Hz, 1H), 7.42 (d, J=2.80 Hz, 1H), 7.39 (s, 1H), 7.31 (d, J=8.80 Hz, 1H), 4.34 (t, J=5.2 Hz, 2H), 4.1 (t, J=5.2 Hz, 2H), 3.45 (s, 2H), 3.33 (q, J=10 Hz, 1H), 2.91 (t, J=5.6, 2H), 2.76 (s, 3H), 2.54 (s, 4H), 1.79 (s, 3H) ppm.

### Synthesis of 5 (Compound 25)

To a stirred solution of the compounds 7-[5-chloranyl-2-[2-[2-methyl-4-oxidanylidene-6-[2,2,2-tris-(fluoranyl)ethyl]-7,8-dihydro-5H-pyrido[4,3-d]pyrimidin-3-yl]ethoxy]phenyl]-5-methyl-thieno[3,2-b]pyridine-3-carboxylic acid (4) (Compound 24) (140 mg, 141.65 μmol) and MeSO2NH₂ (33.68 mg, 354.12 μmol) in DCM (4 mL) were added DMAP (34.61 mg, 283.30 μmol) followed by EDC·HCl (54.97 mg, 354.12 μmol) at 0° C. and the reaction mixture was stirred at 25° C. for 16 h. After completion of the reaction (as judged by LC/MS only), the solvents were evaporated under reduced pressure. The crude thus obtained was purified by reverse phase prep-HPLC to get the 7-[5-chloranyl-2-[2-[2-methyl-4-oxidanylidene-6-[2,2,2-tris(fluoranyl)ethyl]-7,8-dihydro-5H-pyrdo[4,3-d]pyrimidin-3-yl]ethoxy]phenyl]-5-methyl-N-methylsulfonyl-thieno[3,2-b]pyridine-3-carboxamide; 2,2,2-tris(fluoranyl) acetic acid (5) (Compound 25) (24 mg, 29.68 μmol, 20.95% yield, 96.96% purity) as an off white solid.

¹H NMR (400 MHz, DMSO-d6): δ 13.05 (brs, 1H), 8.95 (s, 1H), 7.59 (dd, J=9.20, 2.80 Hz, 1H), 7.48 (s, 1H), 7.45 (d, J=2.80 Hz, 1H), 7.34 (d, J=8.80 Hz, 1H), 4.33 (t, J=4.8 Hz, 2H), 4.09 (t, J=4.8 Hz, 2H), 3.50 (s, 3H), 3.39 (s, 2H), 3.33 (q, J=10 Hz, 2H), 2.91 (t, J=5.2, 2H), 2.74 (s, 3H), 2.54 (s, 2H), 1.64 (s, 3H) ppm.

m/z calc. 569.1, found (M+1)=570.0 at RT 2.68.

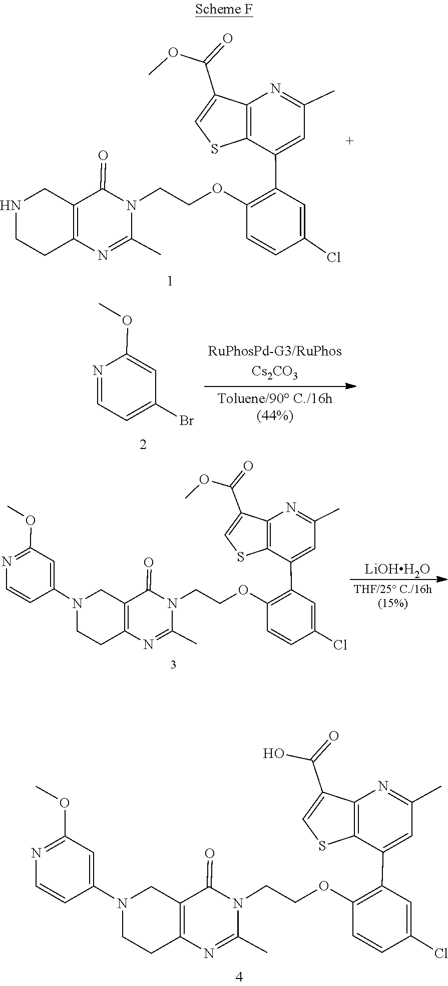

### Synthesis of 3:

To a degassed solution of methyl 7-[5-chloranyl-2-[2-(2-methyl-4-oxidanylidene-5,6,7,8-tetrahydropyrido[4,3-d]pyrimidin-3-yl) ethoxy]phenyl]-5-methyl-thieno[3,2-b]pyridine-3-carboxylate (1) (200 mg, 380.94 μmol) in toluene (5 mL) were 4-bromanyl-2-methoxy-pyridine (2) (107.44 mg, 571.41 μmol) followed by Cs₂CO₃ (496.74 mg, 1.52 mmol) at 25° C. To this were added RuPhos (17.75 mg, 38.09 μmol) followed by RuPhosPdG3 (32.43 mg, 38.09 μmol) at 25° C. and the reaction mixture was degassed with argon for 10 min. The reaction mixture was heated at 90° C. for 16 h. After completion of reaction (as judged by TLC & LC/MS), the reaction mixture was cooled to 25° C. and filtered through celite pad. The filtrate was diluted with water and extracted with EtOAc. The combined organic layer was washed with brine, dried over anhydrous Na₂SO₄, filtered and filtrate was evaporated under reduced pressure. The crude was thus obtained was purified by silica gel combi flash column chromatography (SiO₂; 12 g, 4% MeOH/DCM) to get methyl 7-[5-chloranyl-2-[2-[6-(2-methoxy-4-pyridyl)-2-methyl-4-oxidanylidene-7,8-dihydro-5H-pyrido[4,3-d]pyrimidin-3-yl]ethoxy]phenyl]-5-methyl-thieno[3,2-b]pyridine-3-carboxylate (3) (140 mg, 168.32 μmol, 44.19% yield, 76% purity) as a light yellow solid.

m/z calc. 631.08, found (M+1)=632.0 at RT 3.24.

### Synthesis of 4 (Compound 23)

To a stirred solution of the compound methyl 7-[5-chloranyl-2-[2-[6-(2-methoxy-4-pyridyl)-2-methyl-4-oxidanylidene-7,8-dihydro-5H-pyrido[4,3-d]pyrimidin-3-yl]ethoxy]phenyl]-5-methyl-thieno[3,2-b]pyridine-3-carboxylate (3) (140 mg, 168.32 μmol) in a mixture of THF (4.00 mL) and water (999.77 μL) was added LiOH·H₂O (28.25 mg, 673.28 μmol, 18.71 μL) at 25° C. and the reaction mixture was stirred at 25° C. for 16 h. After completion of reaction (as judged by TLC & LC/MS), the reaction mixture was evaporated under reduced pressure. The crude thus obtained was purified by reverse phase prep-HPLC to afford 7-[5-chloranyl-2-[2-[6-(2-methoxy-4-pyridyl)-2-methyl-4-oxidanylidene-7,8-dihydro-5H-pyrido[4,3-d]pyrimidin-3-yl]ethoxy]phenyl]-5-methyl-thieno[3,2-b]pyridine-3-carboxylic acid; 2,2,2-tris(fluoranyl) acetic acid (4) (Compound 23) (19 mg, 25.07 μmol, 14.90% yield, 96.62% purity) as a white solid.

¹H NMR (400 MHz, DMSO-d6): δ 8.71 (s, 1H), 7.87 (d, J=6.8 Hz, 1H), 7.56 (dd, J=8.8, 2.0 Hz, 1H), 7.42 (d, J=2.4 Hz, 1H), 7.39 (s, 1H), 7.32 (d, J=8.8 Hz, 1H), 6.78 (d, J=6 Hz, 1H), 6.38 (s, 1H), 4.38 (t, J=4.8 Hz, 2H), 4.25 (s, 2H), 4.17 (t, J=4.8 Hz, 2H), 3.99 (s, 3H), 3.78 (t, J=5.6 Hz, 2H), 2.75 (s, 3H), 2.64 (t, J=5.6 Hz, 2H), 1.79 (s, 3H) ppm.

m/z calc. 617.15, found (M-1)=616.1 at RT 2.37.

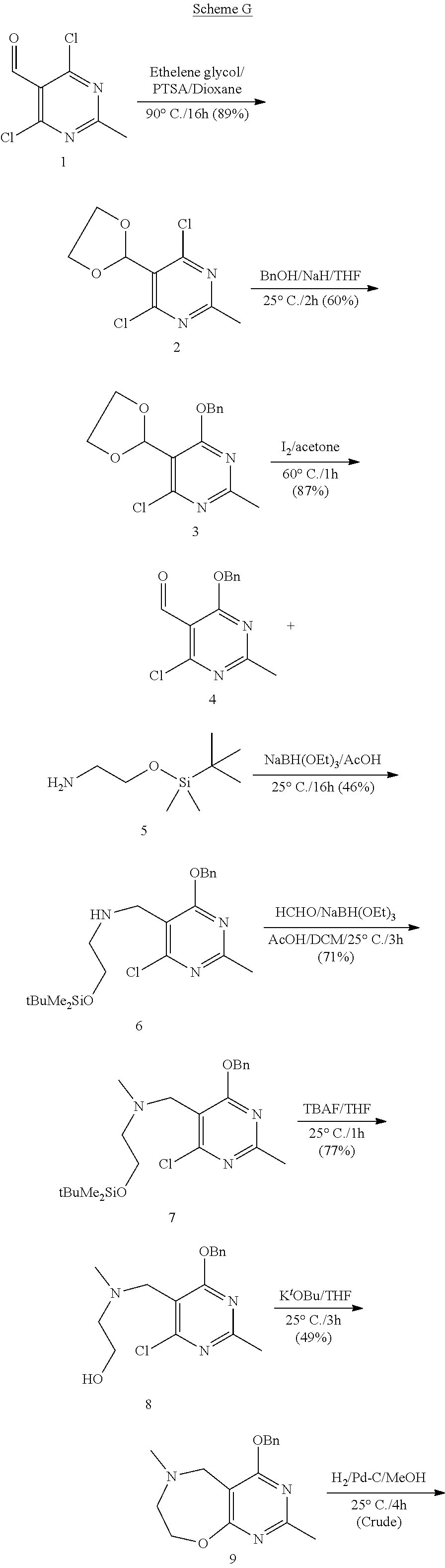

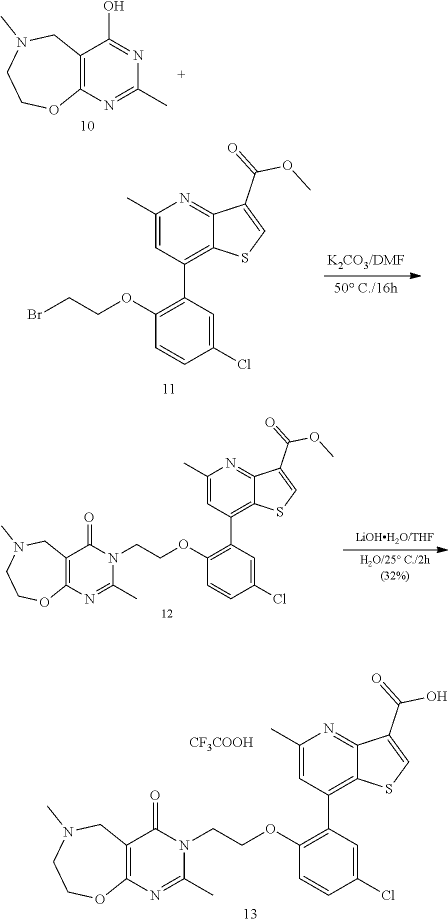

### Synthesis of Compound 2:

To a stirred solution of 4,6-bis(chloranil)-2-methyl-pyrimidine-5-carbaldehyde (5 g, 26.18 mmol) in 1,4-dioxane (20 mL) were added PTSA (450.75 mg, 2.62 mmol) followed by ethane-1,2-diol (4.06 g, 65.44 mmol, 3.65 mL) at 25° C. and the reaction mixture was stirred for 16 h at 100° C. After completion of reaction, the reaction mixture was cooled to 25° C. The volatiles were removed under reduced pressure. The crude was purified by combi flash column chromatography (SiO2, 40 g, 4% EtOAc/Hexanes) to afford 4,6-bis(chloranyl)-5-(1,3-dioxolan-2-yl)-2-methyl-pyrimidine (2) (3.1 g, 13.19 mmol, 50.38% yield) as a white solid.

LC-MS: 235.1 (M+H).

1H-NMR (400 MHz, DMSO-d6): § 6.23 (s, 1H), 4.16-4.22 (m, 2H), 4.00-4.06 (m, 2H), 2.60 (s, 3H) ppm.

### Synthesis of Compound 3:

To a stirred solution of benzyl alcohol (1.71 g, 15.83 mmol, 1.64 mL) in THF (9.73 mL) was added NaH (454.78 mg, 19.78 mmol, 60% in oil) at 0° C. under argon atmosphere and the reaction mixture was stirred at 25° C. for 30 min. To this was added 4,6-bis(chloranyl)-5-(1,3-dioxolan-2-yl)-2-methyl-pyrimidine (2) (3.1 g, 13.19 mmol) in THF (5 ml) at 0° C. and the reaction mixture was stirred at 25° C. for 2 h. After completion of reaction (as judged by LC/MS and TLC), the reaction mixture was quenched with ice cold water and extracted with EtOAc, combined organics were washed with ice cold water, brine, dried over Na₂SO₄, filtered and filtrate was concentrated under reduce pressure. The crude was purified by silica gel column chromatography (SiO₂; 230-400 mesh, 40 g, 4% EtOAc/Hexane) to get 4-chloranyl-5-(1,3-dioxolan-2-yl)-2-methyl-6-(phenyl methoxy)pyrimidine (3) (3.5 g, 11.41 mmol, 86.52% yield) as a yellowish-white solid.

MS calculated: 306.1; MS found: 307.1 (M+H).

1H NMR (400 MHz, DMSO-d6): 7.35-7.52 (m, 5H), 6.15 (s, 1H), 5.46 (s, 2H), 4.20 (s, 2H), 3.89 (s, 2H), 2.52 (s, 3H) ppm

### Synthesis of Compound 4:

To a stirred solution of 4-chloranyl-5-(1,3-dioxolan-2-yl)-2-methyl-6-(phenyl methoxy)pyrimidine (3) (2 g, 6.52 mmol) in acetone (8 mL), was added I₂ (166.91 mg, 652.01 μmol) at 25° C., and the reaction mixture was stirred at 60° C. for 1 h. After completion of reaction, the volatiles were removed under reduced pressure, and crude thus obtained was purified by flash column chromatography (SiO2, 230-400mesh, 20% EtOAc/Hexanes) to give 4-chloranyl-2-methyl-6-(phenyl methoxy)pyrimidine-5-carbaldehyde (4) (1.5 g, 5.71 mmol, 87.58% yield) as a colourless sticky gum.

LC-MS: 263.3 (M+H)

1H-NMR (400 MHz, DMSO-d6): δ 10.33 (s, 1H), 7.51 (d, J=6.64 Hz, 2H), 7.40-7.30 (m, 3H), 5.54 (s, 2H), 3.64 (s, 3H) ppm

### Synthesis of Compound 6:

To a stirred solution of 4-chloranyl-2-methyl-6-(phenyl methoxy)pyrimidine-5-carbaldehyde (4) (1.7 g, 6.47 mmol) in DCM (10.40 mL) was added 2-[1,1-di(methyl)ethyl-di(methyl)silyl] oxyethanamine (5) (1.36 g, 7.77 mmol, 1.60 mL) and catalytic amount AcOH at 25° C. and the reaction mixture was stirred for 30 min at 25° C. To this reaction mixture was added sodium triacetoxyborohydride (5.49 g, 25.89 mmol) at 0° C. portion wise and stirring was continued for 16 h at 25° C. After completion of reaction (as judged by TLC & LC/MS), the volatiles were removed under reduced pressure. The crude thus obtained was purified by combi flash column chromatography (SiO2; 12 g, 30% EtOAc/Hexanes) to afford N-[[4-chloranyl-2-methyl-6-(phenyl methoxy)pyrimidin-5-yl]methyl]-2-[1,1-di(methyl)ethyl-di(methyl) silyl]oxy-ethanamine (6) (2 g, 4.74 mmol, 73.23% yield) as an off white solid.

LC-MS: 421.3 (M+H)

1H-NMR (400 MHz, DMSO-d6): δ 7.46 (d, J=8 Hz, 2H), 7.40-7.34 (m, 3H), 5.43 (s, 2H), 3.94 (s, 2H), 3.77 (s, 3H), 2.16 (s, 3H), 0.85 (s, 9H), −0.03 (s, 6H)

### Synthesis of Compound 7:

To a stirred solution of N-[[4-chloranyl-2-methyl-6-(phenyl methoxy)pyrimidin-5-yl]methyl]-2-[1,1-di(methyl)ethyl-di(methyl) silyl]oxy-ethanamine (6) (2 g, 4.74 mmol) in DCM (10.00 mL) was added HCHO (1.61 g, 47.39 mmol, 1.98 mL) and catalytic amount AcOH at 25° C. and the reaction mixture was stirred for 30 min at 25° C. To this reaction mixture was added sodium triacetoxyborohydride (5.02 g, 23.69 mmol) at 0° C. portion wise and stirring was continued for 3 h at 25° C. After completion of reaction (as judged by TLC & LC/MS), the volatiles were removed under reduced pressure. The crude thus obtained was purified by combi flash column chromatography (SiO2; 12 g, 30% EtOAc/Hexanes) to afford N-[[4-chloranyl-2-methyl-6-(phenyl methoxy)pyrimidin-5-yl] methyl]-2-[1,1-di(methyl)ethyl-di(methyl) silyl]oxy-N-methyl-ethanamine (7) (980 mg, 2.25 mmol, 47.42% yield) as an off white solid.

LC-MS: 436.3 (M+H)

1H-NMR (400 MHz, DMSO-d6): δ 7.46 (d, J=8 Hz, 2H), 7.40-7.34 (m, 3H), 5.43 (s, 2H), 3.60 (t, J=12 Hz, 2H), 3.54 (s, 2H), 3.29 (t, J=12 Hz, 2H), 2.50 (s, 3H), 2.16 (s, 3H), 0.85 (s, 9H), −0.034 (s, 6H).

### Synthesis of Compound 8:

To a stirred solution of N-[[4-chloranyl-2-methyl-6-(phenyl methoxy)pyrimidin-5-yl] methyl]-2-[1,1-di(methyl)ethyl-di(methyl)silyl]oxy-N-methyl-ethanamine (7) (1.4 g, 3.21 mmol) in THF (10 mL) was added TBAF (2.52 g, 9.63 mmol, 2.79 mL, 1M in THF) at 0° C. and the reaction mixture was stirred at 25° C. for 6 h. After completion of reaction (as judged by LC/MS and TLC), the reaction mixture was quenched with ice cold water and extracted with EtOAc, combined organics were washed with ice cold water, brine, dried over Na₂SO₄, filtered and filtrate was concentrated under reduce pressure. The crude was purified by combi flash column chromatography (SiO2; 12 g, 40 g, 70% EtOAc/Hexane) to get 2-[[4-chloranyl-2-methyl-6-(phenyl methoxy)pyrimidin-5-yl]methyl-methyl-amino]ethanol (8) (800 mg, 2.49 mmol, 77.43% yield) as an off white solid.

LC-MS: 321.6 (M+H)

1H-NMR (400 MHz, DMSO-d6): δ 7.48 (d, J=8 Hz, 2H), 7.41-7.32 (m, 3H), 5.44 (s, 2H), 4.27 (s, 1H), 3.53 (s, 2H), 3.45 (s, 2H), 3.30 (s, 3H), 2.07 (s, 3H).

### Synthesis of Compound 9:

To a stirred solution of 2-[[4-chloranyl-2-methyl-6-(phenylmethoxy)pyrimidin-5-yl]methyl-methyl-amino]ethanol (8) (800 mg, 2.49 mmol) in THF (5 mL) was added potassium tert-butoxide (278.95 mg, 2.49 mmol) and the reaction mixture was stirred at 25° C. for 1 h. After completion of reaction (as judged by TLC & LC/MS), the volatiles were removed under reduced pressure and the crude thus obtained was purified by combi flash column chromatography (SiO2; 12 g, 40 g, 60% EtOAc/Hexane) to give 2,6-di(methyl)-4-(phenylmethoxy)-7,8-dihydro-5H-pyrimido[5,4-f][1,4]oxazepine (350 mg, 1.23 mmol, 49.34% yield) as an off white solid.

LC-MS: 286.0 (M+H)

1H NMR (400 MHz, DMSO-d6) δ 7.45-7.31 (m, 5H), 5.39 (s, 2H), 4.23-4.17 (m, 3H), 3.63 (s, 3H), 2.86-2.83 (m, 3H), 1.56 (s, 3H) ppm

### Synthesis of Compound 10:

To a stirred solution of 2,6-di(methyl)-4-(phenylmethoxy)-7,8-dihydro-5H-pyrimido[5,4-f][1,4]oxazepane (9) (350 mg, 1.23 mmol) in MeOH (5 mL) was added 10% Palladium on carbon wet (130.54 mg, 1.23 mmol) and the reaction mixture was degassed with argon for 30 min, the reaction mixture was stirred under positive pressure of H₂ for 3 h at 25° C. After completion of reaction, the catalysis was filtered by sintered funnel. The filtrate were removed under reduced pressure and the crude thus obtained was purified by combi flash column chromatography (SiO₂; 4 g, 40 g, 50% EtOAc/Hexane) to get 2,6-di(methyl)-7,8-dihydro-5H-pyrimido[5,4-f][1,4]oxazepin-4-ol (10) (210 mg, 1.08 mmol, 87.70% yield) as a white solid.

LC-MS: 196.2 (M+H)

1H NMR (400 MHz, DMSO-d6) δ 12.28-12.25 (m, 1H), 4.16-4.14 (m, 2H), 3.58 (s, 1H), 3.45 (s, 1H), 2.77-2.74 (m, 2H), 2.28 (s, 3H), 2.18 (s, 2H), 1.55 (s, 2H) ppm

### Synthesis of Compound 12:

To a stirred solution of 2,6-di(methyl)-7,8-dihydro-5H-pyrimido[5,4-f][1,4]oxazepin-4-ol (170 mg, 870.82 μmol) (10) in DMF (3 mL) was added potassium carbonate, anhydrous, 99% (361.07 mg, 2.61 mmol, 157.67 μL) followed by methyl 7-[2-(2-bromanylethoxy)-5-chloranyl-phenyl]-5-methyl-thieno[3,2-b]pyridine-3-carboxylate (11) (460.57 mg, 1.04 mmol) at 25° C. and the reaction mixture was stirred at 50° C. for 16 h. After completion of reaction (as judged by LC/MS and TLC), the reaction mixture was quenched with ice cold water and extracted with EtOAc, combined organics were washed with ice cold water, brine, dried over Na₂SO₄, filtered and filtrate was concentrated under reduce pressure. The crude was purified by combi flash column chromatography (SiO2; 12 g, 40 g, 60% EtOAc/Hexane) to get methyl 7-[5-chloranyl-2-[2-[2,6-di(methyl)-4-oxidanylidene-7,8-dihydro-5H-pyrimido[5,4-f][1,4]oxazepin-3-yl]ethoxy]phenyl]-5-methyl-thieno[3,2-b]pyridine-3-carboxylate (12) (110 mg, 198.18 μmol, 22.76% yield) as an off white solid & methyl 7-[5-chloranyl-2-[2-[[2,6-di(methyl)-7,8-dihydro-5H-pyrimido[5,4-f][1,4]oxazepin-4-yl]oxy]ethoxy]phenyl]-5-methyl-thieno[3,2-b]pyridine-3-carboxylate (13) (117 mg, 210.79 μmol, 24.21% yield) as an off white solid.

LC-MS: 556.3 (M+H)

1H-NMR (400 MHz, DMSO-d6): δ 8.75 (s, 1H), 7.54 (d, J=4 Hz, 1H), 7.41 (s, 1H), 7.27 (t, J=12 Hz, 2H), 4.26 (d, J=8 Hz, 2H), 4.17 (s, 2H), 4.03 (d, J=8 Hz, 4H), 3.88 (s, 3H), 3.45 (s, 2H), 2.76 (s, 2H), 1.55 (s, 3H), 1.23 (s, 3H).

### Synthesis of 13 (Compound 30)

To a stirred solution of methyl 7-[5-chloranyl-2-[2-[2,6-di(methyl)-4-oxidanylidene-7,8-dihydro-5H-pyrimido[5,4-f][1,4]oxazepin-3-yl]ethoxy]phenyl]-5-methyl-thieno[3,2-b]pyridine-3-carboxylate (12) (110 mg, 198.18 μmol) in THF (3 mL) & water (1 ml) was added lithium hydroxide monohydrate, 98% (24.95 mg, 594.55 μmol, 16.52 μL) at 25° C. and the reaction mixture was stirred at 25° C. for 2 h. After completion of reaction (as judged by TLC & LC/MS), the volatiles were removed under reduced pressure. The crude thus obtained was purified by reverse phase prep-HPLC to get 7-[5-chloranyl-2-[2-[2,6-di(methyl)-4-oxidanylidene-7,8-dihydro-5H-pyrimido[5,4-f][1,4]oxazepin-3-yl]ethoxy]phenyl]-5-methyl-thieno[3,2-b]pyridine-3-carboxylic acid; 2,2,2-tris(fluorenyl) acetic acid (13) (Compound 30) (42 mg, 63.83 μmol, 32.21% yield, 99.55% purity) as a white solid.

(M+H)=541.1.

1H NMR (400 MHz, DMSO-d6) δ 8.88 (s, 1H), 7.58 (dd, J=4 Hz, 4 Hz, 1H), 7.47 (s, 1H), 7.41 (s, 1H), 7.31 (d, J=8 Hz, 1H), 4.37 (d, J=16 Hz, 1H), 4.29 (s, 2H), 4.10 (s, 3H), 4.07 (s, 2H), 3.69 (s, 1H), 2.89 (s, 3H), 2.73 (s, 3H), 1.60 (s, 3H).

### II. Biological Assessment

### Biochemical Competitive Binding Assay

The potencies of compounds binding to human eIF4E protein were measured using a 384-well time-resolved fluorescence resonance energy transfer (TR-FRET) competition assay. The assay was performed in assay buffer containing 50 mM HEPES pH 7.5, 100 mM KCl, 0.02% Tween-20, and 0.1 mg/mL bovine serum albumin (BSA). The reaction volume was 10 μL and each reaction contained 4 nM recombinant 6×HIS-tagged human eIF4E protein (Novus, NBP-45314), 5 nM EDA-m7GDP-ATTO-647N (Jena Bioscience, NU-827-647N), 2.5 nM Europium conjugated Anti-6×HIS antibody (PerkinElmer, AD0402) and varying concentrations of compound. The final DMSO concentration was 1%.

Compounds were prepared using 11-point, 4-fold serial dilutions in DMSO and 100 nL of diluted compounds were transferred to 384-well assay-ready plates. Recombinant human eIF4E protein at 2× final concentration (8 nM) was pre-incubated with Eu-anti-6×HIS antibody at 2× final concentration (5 nM) for 5 minutes, then 5 μL of the protein solution was added to assay-ready plates. The protein/compound mix was incubated for 15 minutes, after which 5 μL of a solution containing EDA-m7GDP-ATTO-647N probe at 2× final concentration (10 nM) was added. After a subsequent 15-minute incubation, time-resolved fluorescence of assay plates was measured using a Clariostar Plus microplate reader (BMG Labtech) and TR-FRET values were calculated by taking a ratio of the 665 to 620 wavelength signals. After normalization to the average of DMSO control and maximum inhibition control wells, concentration-response data were plotted and standard 4-parameter curve fitting (PEI Signals) was utilized to determine IC₅₀ values. The results of the competitive binding assay are presented in Table E1.

TABLE E1

| | hEIF4E |
|---|---|
| Compound No. | TR-FRET IC₅₀ |
| 1 | A |
| 2 | C |
| 3 | C |
| 4 | A |
| 5 | B |
| 6 | B |
| 7 | B |
| 8 | A |
| 9 | A |
| 10 | A |
| 11 | C |
| 12 | A |
| 13 | A |
| 14 | A |
| 15 | B |
| 16 | A |
| 17 | B |
| 18 | A |
| 19 | A |
| 20 | A |
| 21 | A |
| 22 | A |
| 23 | A |
| 24 | A |
| 25 | A |
| 26 | A |
| 27 | A |
| 28 | A |
| 29 | A |
| 30 | A |
| 31 | C |

A = (IC₅₀ < 400 nM);
B = (400 nM < IC₅₀ < 10 μM);
C = (10 μM < IC₅₀ < 100 μM)

### Cellular Cap-Dependent Translation Inhibition Assay

The effect of compounds on cellular cap-dependent translation was assessed using a stably integrated Flp-In™-293 (ThermoFisher Scientific, R75007) reporter cell-based dual luciferase assay (DLA), wherein the cap-dependent translation of unstable Firefly luciferase (Fluc-PEST) and cap-independent, poliovirus IRES-mediated translation of Renilla luciferase (Rluc) are measured after 24 hours of compound treatment. The reporter plasmid was constructed utilizing pcDNA5/FRT (Invitrogen, V601020). Test compounds were prepared using 11-point, 4-fold serial dilutions in DMSO and 100 nL of diluted compounds were transferred to 384-well assay-ready plates. Reporter cells were seeded to assay-ready plates in DMEM medium supplemented with 10% FBS at 10,000 cells in a volume of 33.5 microliters per well. After 24 hours of compound treatment, Fluc and Rluc activities were assessed sequentially using the Dual-Glo® Luciferase assay system (Promega, E2920) according to the manufacturer's instructions. Luminescence was measured using a Clariostar Plus microplate reader (BMG Labtech) or equivalent. For each well, the ratio of Fluc luminescence to Rluc luminescence was calculated. After normalization to the average of DMSO control and maximum inhibition control wells, concentration-response data were plotted and standard 4-parameter curve fitting (PEI Signals) was utilized to determine IC₅₀ values. Results of the cellular cap-dependent translation dual luciferase inhibition assay (DLA) are summarized in Table E2.

TABLE E2

| Compound | DLA IC₅₀ |
|---|---|
| 4 | B |
| 5 | C |
| 8 | A |
| 9 | C |
| 10 | C |
| 11 | C |
| 12 | C |
| 13 | C |
| 14 | A |
| 15 | C |
| 16 | C |
| 17 | C |
| 18 | A |
| 19 | A |
| 20 | A |
| 21 | A |
| 22 | B |
| 23 | A |
| 24 | A |
| 25 | A |
| 26 | A |
| 27 | A |
| 28 | A |
| 29 | A |
| 30 | A |
| 31 | C |

A = (IC₅₀ < 10 μM);
B = (10 μM < IC₅₀ < 20 μM);
C = (IC₅₀ > 20 μM)

### EQUIVALENTS

As used herein and in the appended claims, the singular forms “a,” “an,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth.

While specific embodiments of the subject disclosure have been discussed, the above specification is illustrative and not restrictive. Many variations of the disclosure will become apparent to those skilled in the art upon review of this specification and the claims below. The full scope of the disclosure should be determined by reference to the claims, along with their full scope of equivalents, and the specification, along with such variations.

What is claimed is:

1. A compound of Formula I: 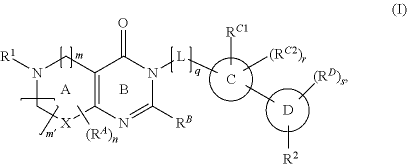
or a pharmaceutically acceptable salt, solvate, stereoisomer, or prodrug thereof,
wherein:
each -L- is independently —O—, —NR^{L}—, —CR^{L1}R^{L2}—, —CR^{L1}═CR^{L2}—, or —C≡C—;
each R^{L} is independently hydrogen or optionally substituted C₁₋₆ alkyl;
each R^{L1} and each R^{L2} is independently hydrogen, halogen, —CN, —NO₂, —OH, —NH₂, C₁₋₆ alkyl, C₁₋₆ alkoxy, or C₁₋₆ alkylamino, wherein the alkyl, alkoxy, or alkylamino is optionally substituted;
q is an integer selected from 1 to 5;
Ring C and Ring D are independently C₆₋₁₀ aryl or 5- to 10-membered heteroaryl;
R^{C1}, each R^{C2}, and each R^{D} are independently halogen, —CN, —NO₂, —OH, —NH₂, C₁₋₆ alkyl, C₂₋₆ alkenyl, C₂₋₆ alkynyl, C₁₋₆ alkoxy, C₁₋₆ alkylamino, C₃₋₆ carbocyclyl, or 3- to 6-membered heterocyclyl, wherein the alkyl, alkenyl, alkynyl, alkoxy, alkylamino, carbocyclyl, or heterocyclyl is optionally substituted;
r and s are independently an integer selected from 0 to 6, as valency permits;
R² is halogen, —CN, —NO₂, —OH, —NH₂, C₁₋₆ alkyl, C₂₋₆ alkenyl, C₂₋₆ alkynyl, C₁₋₆ alkoxy, C₁₋₆ alkylamino, C₆₋₁₀ aryl, 5- to 10-membered heteroaryl, C₃₋₆ carbocyclyl, 3- to 6-membered heterocyclyl, —NR^{c}S(═O)₂R^{a}, —N(S(═O)₂R^{a})₂, —S(═O)₂R^{a}, —S(═O)₂OR^{b}, —S(═O)₂NR^{c}R^{d}, —C(═O)OR^{b}, —C(═O)NR^{c}S(═O)₂R^{a}, or C(═O)NR^{c}R^{d}, wherein the alkyl, alkenyl, alkynyl, alkoxy, alkylamino, carbocyclyl, heterocyclyl, aryl, or heteroaryl is optionally substituted;
R¹ is hydrogen, —CN, C₁₋₆ alkyl, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —(C₁₋₆ alkylene)-(C₆₋₁₀ aryl), (C₁₋₆ alkylene)-(5- to 10-membered heteroaryl), —(C₁₋₆ alkylene)-(C₃₋₁₂ carbocyclyl), —(C₁-6 alkylene)-(3- to 12-membered heterocyclyl), —S(═O)R^{a}, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, alkylene, carbocyclyl, heterocyclyl, aryl, or heteroaryl is optionally substituted;
X is —O— or —C(R^{X})₂—;
each R^{X} is independently hydrogen, halogen, —CN, —NO₂, —OH, —NH₂, C₁₋₆ alkyl, C₂₋₆ alkenyl, C₂₋₆ alkynyl, C₁₋₆ alkoxy, C₁₋₆ alkylamino, C₃₋₆ carbocyclyl, or 3- to 6-membered heterocyclyl, wherein the alkyl, alkenyl, alkynyl, alkoxy, alkylamino, carbocyclyl, or heterocyclyl is optionally substituted; or
two geminal R^{X}, together with the carbon atom to which they are connected, form an oxo;
m and m′ are independently an integer selected from 0 to 2;
each R^{A} is independently oxo, halogen, —CN, —NO₂, —OH, —NH₂, C₁₋₆ alkyl, C₂₋₆ alkenyl, C₂₋₆ alkynyl, C₁₋₆ alkoxy, C₁₋₆ alkylamino, C₃₋₆ carbocyclyl, or 3- to 6-membered heterocyclyl, wherein the alkyl, alkenyl, alkynyl, alkoxy, alkylamino, carbocyclyl, or heterocyclyl is optionally substituted;
n is an integer selected from 0 to 10, as valency permits;
R^{B} is hydrogen, halogen, —CN, —NO₂, —OH, —NH₂, C₁₋₆ alkyl, C₂₋₆ alkenyl, C₂₋₆ alkynyl, C₁₋₆ alkoxy, C₁₋₆ alkylamino, C₃₋₆ carbocyclyl, or 3- to 6-membered heterocyclyl, wherein the alkyl, alkenyl, alkynyl, alkoxy, alkylamino, carbocyclyl, or heterocyclyl is optionally substituted;
wherein:
each R^{a} is independently C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl;
each R^{b} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; and
each R^{c} and each R^{d} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; or
R^{c} and R^{d}, together with the nitrogen atom to which they are attached, form 3- to 12-membered heterocyclyl;
wherein each occurrence of R^{a}, R^{b}, R^{c}, and R^{d} is independently and optionally substituted.

2. The compound of claim 1, wherein Ring C is phenyl or pyridinyl.

3. The compound of claim 1 or 2, wherein the compound of Formula I is a compound of Formula I-1-i, I-1-ii, I-1-iii, or I-1-iv: 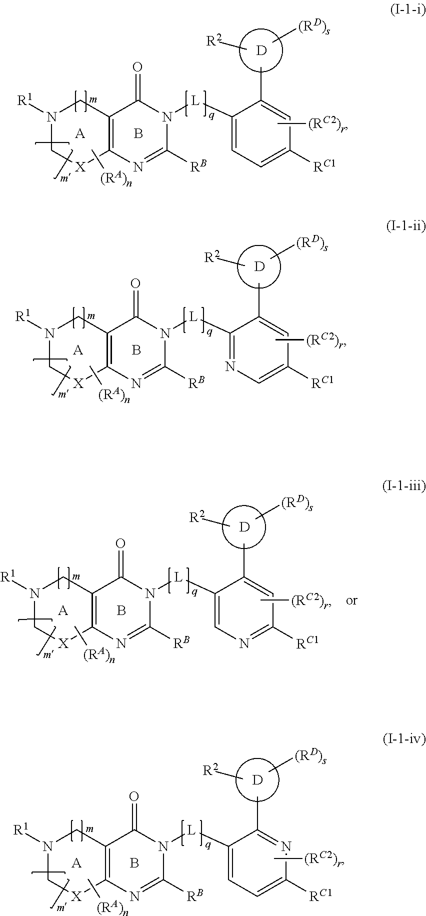
or a pharmaceutically acceptable salt, solvate, stereoisomer, or prodrug thereof.

4. The compound of any one of claims 1-3, wherein Ring D is phenyl, pyridinyl, pyrrolopyridazinyl, or thienopyridinyl.

5. The compound of any one of claims 1-4, wherein R² is halogen, —CN, —NO₂, —OH, —NH₂, C₁₋₆ alkyl, C₂₋₆ alkenyl, C₂₋₆ alkynyl, C₁₋₆ alkoxy, C₁₋₆ alkylamino, C₆₋₁₀ aryl, 5- to 10-membered heteroaryl, C₃₋₆ carbocyclyl, 3- to 6-membered heterocyclyl, —NR^{c}S(═O)₂R^{a}, —N(S(═O)₂R^{a})₂, —S(═O)₂R^{a}, —S(═O)₂OR^{b}, —S(═O)₂NR^{c}R^{d}, —C(═O)OR^{b}, —C(═O)NR^{c}S(═O)₂R^{a}, or —C(═O)NR^{c}R^{d}, wherein the alkyl, alkenyl, alkynyl, alkoxy, alkylamino, carbocyclyl, heterocyclyl, aryl, or heteroaryl is optionally substituted.

6. The compound of any one of claims 1-5, wherein R² is —C(═O)OR^{b} or —C(═O)NR^{c}S(═O)₂R^{a}.

7. The compound of any one of claims 1-6, wherein R² is —COOH or —C(═O)NR^{c}S(═O)₂CH₃.

8. The compound of claim 1, wherein the compound of Formula I is a compound of Formula I-1-i-1, I-1-i-2, I-1-i-3, I-1-iii-1, I-1-iii-2, or I-1-iii-3: 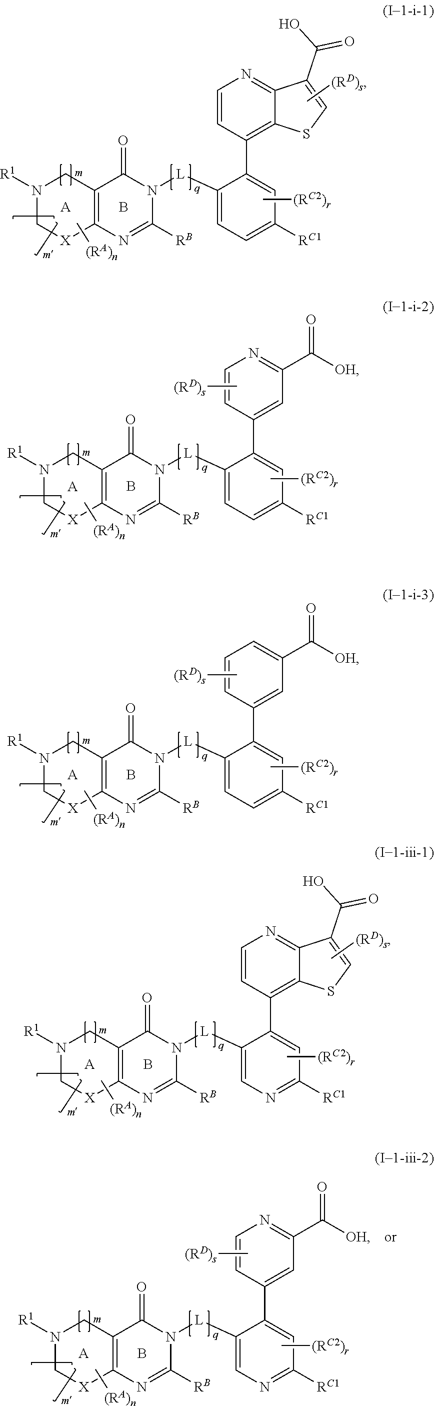 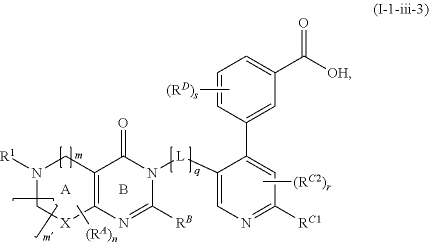
or a pharmaceutically acceptable salt, solvate, stereoisomer, or prodrug thereof.

9. The compound of any one of claims 1-8, wherein R^{C1} is halogen, —CN, —NO₂, —OH, —NH₂, C₁₋₆ alkyl, C₂₋₆ alkenyl, C₂₋₆ alkynyl, C₁₋₆ alkoxy, C₁₋₆ alkylamino, C₃₋₆ carbocyclyl, or 3- to 6-membered heterocyclyl, wherein the alkyl, alkenyl, alkynyl, alkoxy, alkylamino, carbocyclyl, or heterocyclyl is optionally substituted.

10. The compound of any one of claims 1-9, wherein R^{C1} is halogen or C₁₋₆ alkyl.

11. The compound of any one of claims 1-10, wherein R^{C1} is —Cl, —F, or methyl.

12. The compound of any one of claims 1-11, wherein r is 0.

13. The compound of any one of claims 1-12, wherein each R^{D} is independently halogen, —CN, —NO₂, —OH, —NH₂, C₁₋₆ alkyl, C₂₋₆ alkenyl, C₂₋₆ alkynyl, C₁₋₆ alkoxy, C₁₋₆ alkylamino, C₃₋₆ carbocyclyl, or 3- to 6-membered heterocyclyl, wherein the alkyl, alkenyl, alkynyl, alkoxy, alkylamino, carbocyclyl, or heterocyclyl is optionally substituted.

14. The compound of any one of claims 1-13, wherein s is 0 or 1.

15. The compound of any one of claims 1-13, wherein [L]_{q} is 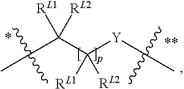
wherein:
* denotes attachment to Ring B, and ** denotes attachment to Ring C;
p is an integer selected from 0 to 3; and
Y is —O—, —NR^{L}—, —CR^{L1}R^{L2}—, or —C≡C—.

16. The compound of claim 15, wherein each R^{L1} and each R^{L2} is hydrogen.

17. The compound of claim 15 or 16, wherein p is 1.

18. The compound of any one of claims 15-17, wherein Y is —O—.

19. The compound of any one of claims 1-18, wherein X is —C(R^{X})₂—.

20. The compound of any one of claims 1-19, wherein each of m and m′ is 1.

21. The compound of any one of claims 1-20, wherein n is 0.

22. The compound of any one of claims 1-21, wherein R^{B} is hydrogen, halogen, —CN, —NO₂, —OH, —NH₂, C₁₋₆ alkyl, C₂₋₆ alkenyl, C₂₋₆ alkynyl, C₁₋₆ alkoxy, C₁₋₆ alkylamino, C₃₋₆ carbocyclyl, or 3- to 6-membered heterocyclyl, wherein the alkyl, alkenyl, alkynyl, alkoxy, alkylamino, carbocyclyl, or heterocyclyl is optionally substituted.

23. The compound of any one of claims 1-22, wherein R¹ is hydrogen, —CN, C₁₋₆ alkyl, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —(C₁₋₆ alkylene)-(C₆₋₁₀ aryl), —(C₁₋₆ alkylene)-(5- to 10-membered heteroaryl), —(C₁₋₆ alkylene)-(C₃₋₁₂ carbocyclyl), —(C₁₋₆ alkylene)-(3- to 12-membered heterocyclyl), —S(═O)R^{a}, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, alkylene, carbocyclyl, heterocyclyl, aryl, or heteroaryl is optionally substituted.

24. A compound selected from Table 1, or a pharmaceutically acceptable salt, solvate, stereoisomer, or prodrug thereof.

25. A pharmaceutical composition comprising the compound of any one of claims 1-24, and a pharmaceutically acceptable excipient.

26. A method of inhibiting a protein in a subject or biological sample, comprising administering the compound of any one of claims 1-24 to the subject or contacting the biological sample with the compound of any one of claims 1-24.

27. Use of the compound of any one of claims 1-24 in the manufacture of a medicament for inhibiting a protein in a subject or biological sample.

28. A compound of any one of claims 1-24 for use in inhibiting a protein in a subject or biological sample.

29. The method, use, or compound for use of any one of claims 26-28, wherein the protein is eIF4E.

30. A method of treating or preventing a disease or disorder a subject in need thereof, comprising administering to the subject the compound of any one of claims 1-24.

31. Use of the compound of any one of claims 1-24 in the manufacture of a medicament for treating or preventing a disease or disorder in a subject in need thereof.

32. A compound of any one of claims 1-24 for use in treating or preventing a disease or disorder in a subject in need thereof.

33. The method, use, or compound for use of any one of claims 30-32, wherein the disease or disorder is an eIF4E-mediated disease or disorder.
